SEQUENCE LISTING (73 sequences)

SEQ ID NO: 1 - DNA, 10140 bp, Deformed Wing Virus
gcatagcgaattacggtgcaactaacaattttagatagtagccatgaacaaacattatgattactcactacgtattgatcatttttacaatgacttgcgtagcatgaagcgcatgcttgtagttataactatgttattttgcaagttggagataattgtattggattatggatgcgtgcactaagtgtctacatctatagtcgtttgtggttcaagtttttgtgttagtagtacaatcttgaagaatgtaagtatcgtatgaatgatatttgtatgacaacactgaagtataaaatatataaaatccaaaaatatttttaattttattcagtgtagtgtttgatagagtagaatgccatgtgaccgctcaaggaagtccattatggtatatcattcgaagtcgaatacttgtgtatagttattgtattttattagtaatattagtagtccgtaactatcataatcctattatagtttgattatatgatagaccactgcagtatcgagtagagtttagaaagagtagtgcaatagtaagatcactgtcaccgaccactcattgtaatagtgaggtttgtcggaaaccagttattgtgcagcgactagcaatcgtgaatcaatatagttggtattctaaatatgagacgattcggcgattttattgcgactgaaatttcatatttagcatgtcaggtcttattatgaatgctcgagtatttatttctgcggtagagtagggacccctctatctctcaggtactgtatgaggcgaaagtgtgaaagtaatttatgtctctatacataagtgactgtatcgggatttcctttggcaagaatccttttaatacagtataatttatgctacggtacgttacgttcgcagggcacccgttaatgtcacatagcccagacgatgacgaatggaaagacattactttttattttaatgctacgattattgctgttttattttgctgtttttatttgctattatattttgctttttcattattgctaaatatatttctttgctatttttgctttatatattagattcaattctttttattttatattttcaatttgattttgattttgaaggtaaatatatataaaaatggcatttagttgtggaactctttcttatgctgctgttgcccaagctccctctgtagctcatgctccccgtagttgggagattgatgaagctaggcgtcgacgcgttatcaagcgtttggcgttggaacaggaacggattcgaaacgttcttgacgttgatgtctatgcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagcgcatcagcgagtaatgaagcaaaattagtgataccttttaaacacgtatatccattcttaccaacgcgtgtcgttcctgattggacaactggtattcttgatatgggtaccttaaatattcgtgtaattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacccggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagtccaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaaccaggtatttgtgtttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataatcggtataactgcgtatgaaatgagaaatccgaaaccaacttctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattttacagaacatggtttacattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcgggatttacaacggccaattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatctgatattggtttagataccgatttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagacgaaatgaagcgagtaatgtggaccatggcgcaggagattttagcgcctagtcatttatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagagaatgctaagcaagcgctagagttagcattcggatggggtccagaatactttaactatgtcagaaatactattaaaatggcttttgacaagttgggtatttatgaagaccttatcacatgggaagaaatggatgttagatgttatgctagcgcgtagtatttaattttgaatacttattagttttaattttattttaggttattggaattgagggaagtaccaccccccaagaccttcgttttaaatctactaaaaggagtgaacctatatataagagtctaacgacagagtggatcagaccaccatctttagcttatatatgggaaaggttgagttgcctctaaagactcagctccatagtagagtagttttaattacgattaaagtggtactctaggttaggtgttactcgcgtattatcaactagtggtaatgcgtcctaattttagtatagttttaaccataatagtaaaaaaaaaaaaaaaaaaaaaaaaaaa

SEQ ID NO: 2 - DNA, 10167 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctttagtatgaatgttatagaatgacagtatcgaaggaaaaatttttataaaatacaaaaatattgttcttattatttcgatacggtgttttatagagtagattgccatgtgaccgctcatagaagtccattatggtttatcaatcgaagttgaatgtatttataaggatattatacttaattagtaatattagtagtccgtaactattatcatcctttttcagtttgatgtgataatagaccactgcagtatcgagtagagtttcgaatgcgtagtgcaatagtataatcactgtcaccgaccatctattgtaataatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaatttccattattgcgaaatatattacattgctatttttattatatacgctagattcaattttattttttctatattttcaatttaattttgatttcgaaggtaaatatatataattaattattaaaaatggcatttagttgtggaactctttcttatgctgctgttgcccaagctccctctgtagctcatgctccccgtagttgggagattgatgaagctaggcgtcgacgcgttatcaagcgtttggcgttggaacaggaacggattcgaaacgttcttgacgttgatgtctatgcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagcgcatcagcgagtaatgaagcaaaattagtgataccttttaaacacgtatatccattcttaccaacgcgtgtcgttcctgattggacaactggtattcttgatatgggtaccttaaatattcgtgtaattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacccggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagtccaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaaccaggtatttgtgtttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataatcggtataactgcgtatgaaatgagaaatccgaaaccaacttctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattttacagaacatggtttacattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcgggatttacaacggccaattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatctgatattggtttagataccgatttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagacgaaatgaagcgagtaatgtggaccatggcgcaggagattttagcgcctagtcatttatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagagaatgctaagcaagcgctagagttagcattcggatggggtccagaatactttaactatgtcagaaatactattaaaatggcttttgacaagttgggtatttatgaagaccttatcacatgggaagaaatggatgttagatgttatgctagcgcgtagtatttaattttgaatacttattagttttaattttattttaggttattggaattgagggaagtaccaccccccaagaccttcgttttaaatctactaaaaggagtgaacctatatataagagtctaacgacagagtggatcagaccaccatctttagcttatatatgggaaaggttgagttgcctctaaagactcagctccatagtagagtagttttaattacgattaaagtggtactctaggttaggtgttactcgcgtattatcaactagtggtaatgcgtcctaattttagtatagttttaaccataatagtaaaaaaaaaaaaaaaaaaaaaaaaaaa

SEQ ID NO: 3 - DNA, 3477 bp, Varroa-destructor virus-1
atggcatttagttgtggaactctttcttatgctgctgttgcccaagctccctctgtagctcatgctccccgtagttgggagattgatgaagctaggcgtcgacgcgttatcaagcgtttggcgttggaacaggaacggattcgaaacgttcttgacgttgatgtctatgcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagcgcatcagcgagtaatgaagcaaaattagtgataccttttaaacacgtatatccattcttaccaacgcgtgtcgttcctgattggacaactggtattcttgatatgggtaccttaaatattcgtgtaattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacccggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgag

SEQ ID NO: 4 - DNA, 5205 bp, Deformed Wing Virus
ggccctatagcgaagataaagaactttttccaccaaacggcagatgaagttcgagaagctcaggccgcaaagatgcgtgaagatatgggtatagtagtccaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaaccaggtatttgtgtttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataatcggtataactgcgtatgaaatgagaaatccgaaaccaacttctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattttacagaacatggtttacattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcgggatttacaacggccaattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatctgatattggtttagataccgatttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagacgaaatgaagcgagtaatgtggaccatggcgcaggagattttagcgcctagtcatttatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagagaatgctaagcaagcgctagagttagcattcggatggggtccagaatactttaactatgtcagaaatactattaaaatggcttttgacaagttgggtatttatgaagaccttatcacatgggaagaaatggatgttagatgttatgctagcgcgtag

SEQ ID NO: 5 - DNA, 101 bp, Artificial Sequence
  Sequence comprised in the preferred polynucleotides of the invention.
tagtagtccaagacgttataggagagttaagtcaggctatacccgatcttcaacaaccggaagttcaagcgaatgttttttctctggtgtcacagttagtg

SEQ ID NO: 6 - DNA, 350 bp, Artificial Sequence
  Sequence comprised in the preferred polynucleotides of the invention.
ttcaattatcaacgacacagttaatgaggaaaaagggaataaaacctcacactatattcacggattgtttgaaagatacttgtttgcctgttgaaaaatgtagaatacctggtaagactagaatatttagcataagtccggtgcagtttaccataccgtttcgacagtattatttagactttatggcatcctatcgagctgcacgacttaatgctgagcatggtattggtattgatgttaacagcttagagtggacaaatttggcaacaaggttgtctaagtatggcactcacatcgtgacaggagactataagaattttggtcctgggttagattccgatgttgcagct

SEQ ID NO: 7 - DNA, 11040 bp, Artificial Sequence
  pTYF-mCMW/SYN-EGFP
cggactagtcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcagtagtttaacacattatacacttaaaaattggatctacttattcaagatcggccgctggagctgatctgactcagcagagcagagaagtccatgtccgcaatggaggagaagtcttcatctccggagagaccattgggaagccccgaggtccccaggggtgtgggagctgggtcagggggcctctgggaccctgtcaccaggcgagttatagcttcagggtactccatcagcatgggctcagctgtggagtgagacatggacacaccctggttcaggagctgctgaaactctgagttgtccacagatgccaggtctgtgaacactcctgggtctgtgctgttgccaagcaaggcccccaagtcttcatcagcatcaaactgcaggtgcagcagggcttccgacagcgtgccttccccagcctgggtgctctttggaacaggtgcagacagggactggggaggacccggggttagaactggggcaggagctgggggctgagccagaggtaccatggctgaggaagggaccatggtctgggcaaggactggggcagaggacggtgctaaggccagggcctggtttgagatctgcccagaaggaaacaccatggtgagctctgcagaagcttctagaattccagcccgggcggatcccaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggctagcttagatcccagcttttgttccctttagtgagggttaatttcgagcttggcgtaatcgctagcggatctgacggttcactaaaccagctctgcttatatagacctcccaccgtacacgcctaacgcgctagccttgcatgcctgcaggtcggagtactgtcctccgagcggagtactgtcctccgagcggagtactgtcctccgagcggagtactgtcctccgagcggagactctagagggacgcgtatcgatggcgccagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgactctagagctgcaggcaagcttggtaccgagctcggatccactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacgcggccgctcccgttatttgcgctctgttcctgttaatcaacctctggattacaaaatttgtgaaagattgactggtattcttaactatgttgctccttttacgctatgtggatacgctgctttaatgcctttgtatcatgctattacttcccgtacggctttcattttctcctccttgtataaatcctggttgctgtctctttatgaggagttgtggcccgttgtcaggcaacgtggcgtggtgtgcactgtgtttgctgacgcaacccccactggttggggcattgccaccacctatcaactcctttccgggactttcgctttccccctccctattgccacggcggaactcattgccgcctgccttgcccgctgctggacaggggctcggctgttgggcactgacaattccgtggtgttgtcggggaagctgacgtcctttccatggctgctcgcctgtgttgccaactggattctgcgcgggacgtccttctgctacgtcccttcggccctcaatccagcggaccttccttcccgcggcctgctgccggttctgcggcctcttccgcgtcttcgccttcgccctcagacgagtcggatctccctttgggccgcctatcgattagggataacagggtaatggtacctttaagaccaatgacttacaaggcagctgtagatcttagccactttttaaaagaaaaggggggactggaagggctaattcactcccagggtctctctggttagaccagatctgagcctgggagctctctggctaactagggaacccactgcttaagcctcaataaagctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagcgcggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcagggtggcccgagagctgcatccggagtatctagatggagttccgcgttacataacttacggtaaatggcccgcctggctgaccgcccaacgacccccgcccattgacgtcaataatgactgatgttcccatagtaacgccaatagggactttccattgacgctaatgggagtttgttttggcaccaaaatcaacgggactttccaaaatgtcgtaataaccccgccccgttgacgcaaatgggcggtaggcgtgtactctagaaggtctatataagcagagctcgtttagtgaaccgtttatactacttatctggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcacggtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcttgcggccggaattcacaaatggcagtattcatccacaattttaaaagaaaaggggggattggggggtacagtgcaggggaaagaatagtagacataatagcaacagacatacaaactaaagaattacaaaaacaaattactaaaaattcnaaaattttcgggtttattacagggacagcagagatccactttgggc

SEQ ID NO: 8 - DNA, 1093 bp, Apis mellifera
tcatgttctttatatacgcatataaatttcaaaaaaatttataattttattgcatagtaaaagatagaaaattttatataaataaattttttctgtttttaattctatgctattttaaataaaatctgataatacatttcttattatttaaatactattaaataataaaaattttgatttcgatttaaatactttacatatctatttaaaaatatctctatatattttttcttttttatgaatcatataaaatctttataggacatttcattattacgtttgtgatatattgttatgattcattgatattacttttatgtcaattattttatgtatttactattattaaatctactgttataaaaaaaaaatgtcgattttttaatgataatatgtaaaaaaagtaattattttacttatattaaaaattacatcattaaaaattgaacacgtgtattataaaggttatagatattacaagaattatgggaccttgaaaacgtgcgattcatagccaagcatagatataaattttatatatgcatacatctgtatgtgtaactacacaccttctagaattaagtagtaactagatttttttaacgttctttatgatccacttctgtattatttattgttccagccaaaaattatataaaaaattattttatttgaatgttaataatataaaaaataatttcaaaagtttaaaataatttttatcaaagaatgagatttttaaatatttttatatataaaatacgttaggatcgatcgataaccaaataaaataccactattgatacacgtagttcgtggcgtgtatccagtaatcattgaaatctaacagaaactagtttctgattggttgatagaatgtgctagagtaggaagaacattccagaagcgtcctctgggaaggtaatatatacagagagccggatgcgggctcttcactcacgttgcgaatcgtcaatcgaagaacttcttctctgtcctacgaagcgatttttacaaagcgaatttttaccaagcgaacaaagaagaaaggtgtgttcccattacctttgtttgacatctataatatttgata

SEQ ID NO: 9 - DNA, 387 bp, Apis mellifera
ctcgcttgcccactgccctctccatgatttactccgttgcaaaggataacaacttcaagaacatagaattagacagcttcggtctttcgttcctaaatatataatttatctaggttttataattttttttatatcattgtaaaaatcacttctgatttatttattttgttctactgtaaaactagacaaataatttatttcattagtaatttcctacagattataataaacgtaaaaaaaaactattgtacttatattttgaaaatgtggataaaaaagatcagttgttggttttctatgttcaaaaaactgaaaatataaaacaatccctgaaatacaatttttttataacttattttcttaccttcctattaaatttcaattttc

SEQ ID NO: 10 - DNA, 14539 bp, Artificial Sequence
  Sequence of a transducing vector derived from pTYF-mCMW/SYN-EGFP which comprises a luciferase reporter gene operably linked to the honeybee hsp70 promoter
ggcctagggataacagggtaatgatatctctgaccgctagcatcgtcgacccgggatccgcggactagtcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcagtagtttaacacattatacacttaaaaattggatctacttattcaagatcggccgctggagctgatctgactcagcagagcagagaagtccatgtccgcaatggaggagaagtcttcatctccggagagaccattgggaagccccgaggtccccaggggtgtgggagctgggtcagggggcctctgggaccctgtcaccaggcgagttatagcttcagggtactccatcagcatgggctcagctgtggagtgagacatggacacaccctggttcaggagctgctgaaactctgagttgtccacagatgccaggtctgtgaacactcctgggtctgtgctgttgccaagcaaggcccccaagtcttcatcagcatcaaactgcaggtgcagcagggcttccgacagcgtgccttccccagcctgggtgctctttggaacaggtgcagacagggactggggaggacccggggttagaactggggcaggagctgggggctgagccagaggtaccatggctgaggaagggaccatggtctgggcaaggactggggcagaggacggtgctaaggccagggcctggtttgagatctgcccagaaggaaacaccatggtgagctctgcagaagcttctagaattccagcccgggcggatcccaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggctagcttagatcccagcttttgttccctttagtgagggttaatttcgagcttggcgtaatcgctagcggatctgacggttcactaaaccagctctgcttatatagacctcccaccgtacacgcctaacgcgctagccttgcatgcctgcaggtcggagtactgtcctccgagcggagtactgtcctccgagcggagtactgtcctccgagcggagtactgtcctccgagcggagactctagagggacgcgtatcgatggcgccagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgactctagagctgcaggcaagcttggtaccgagctcggatccactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttacacacgcgtgatgtatacgactcactatagggcgattgggcccgacgtcggtacatgtcatgttctttatatacgcatataaatttcaaaaaaatttataattttattgcatagtaaaagatagaaaattttatataaataaattttttctgtttttaattctatgatattttaaataaaaatctgataatacatttcttattatttaaatactattaaataataaatattttgatttcaatttaaatactttacatatgtatttaaaaatatctctatatattttttcttttttatgaatcatataaaatctttataggacatttcattattacgtttgtgatatattgttatgattcattgatattacttttatgtcaattattttatgtatttattattattaaatctactgttataaaaaaaaatgttgatttttttaatgataatatgtaaaaaagtaattattttacttaaattaaaaattacatcattaaaaattgaacacgtgtattataaaggttatagatattacaagaattatgggaccttgaaaacgtgcgattcatagccaagcatagatataaattttatatatgcatacatctgtatgtgtaactacacaccttctagaattaagtagtaactagatttttttaacgttctttatgatccacttctgtattatttattgttccagccaaaaattatataaaaaattattttatttgaatgtcaataatataaaaaataatttcaaaagtttaaaataatttttatcaaagaatgagatttttaaatatttttatatataaaatacgttaggatcgatcgataaccaaataaaataccactattgatacacgtagttcatggcgtgtatccagtaatcattgaaatctaacagaaactagtttctgattggttgatagaatgtgctagagtaggaagaacattccagaagcgtcctctgggaaggtaatatatacagagagccggatgcgggctcttcactcacgttgcgaatcgtcaatcgaagaacttcttctctgtcctacgaagcgatttttacaaagcgaatttttaccaagcgaacaaagaagaaaggtgtgttcccattacctttgtttgacatctataatatttgatacaacattcatattggaaataatcttgaatttttttttttattattaaatttttatttcatatttttacagaaaagcaaaaatgaccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgagggtcccttcccgggactagcctagggtttaaactaaatcgattactcgcttgcccactgccctctccatgatttactccgttgcaaaggataacaacttcaagaacatagaattagacagcttcggtctttcgttcctaaatatataatttatctaggttttataattttttttatatcattgtaaaaatcacttctgatttatttattttgttctactgtaaaactagacaaataatttatttcattagtaatttcctacagattataataaacgtaaaaaaaaactattgtacttatattttgaaaatgtggataaaaaagatcagttgttggttttctatgttcaaaaaactgaaaatataaaacaatccctgaaatacaatttttttataacttattttcttaccttcctattaaatttcaattttcaatcactagtgaattcgcggccgcctgcaggtcgaccatatgggagagctcccaacgcgttggatgcatagcttgagtattctatagtgtcacctaaatagctggcgtatcagtcatgccgaattctgcagtcgacggtaccgcgggcccgggatccaccggatctagataactgatcataatcagccataccacgcggccgctcccgttatttgcgctctgttcctgttaatcaacctctggattacaaaatttgtgaaagattgactggtattcttaactatgttgctccttttacgctatgtggatacgctgctttaatgcctttgtatcatgctattacttcccgtacggctttcattttctcctccttgtataaatcctggttgctgtctctttatgaggagttgtggcccgttgtcaggcaacgtggcgtggtgtgcactgtgtttgctgacgcaacccccactggttggggcattgccaccacctatcaactcctttccgggactttcgctttccccctccctattgccacggcggaactcattgccgcctgccttgcccgctgctggacaggggctcggctgttgggcactgacaattccgtggtgttgtcggggaagctgacgtcctttccatggctgctcgcctgtgttgccaactggattctgcgcgggacgtccttctgctacgtcccttcggccctcaatccagcggaccttccttcccgcggcctgctgccggttctgcggcctcttccgcgtcttcgccttcgccctcagacgagtcggatctccctttgggccgcctatcgattagggataacagggtaatggtacctttaagaccaatgacttacaaggcagctgtagatcttagccactttttaaaagaaaaggggggactggaagggctaattcactcccagggtctctctggttagaccagatctgagcctgggagctctctggctaactagggaacccactgcttaagcctcaataaagctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagcgcggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcagggtggcccgagagctgcatccggagtatctagatggagttccgcgttacataacttacggtaaatggcccgcctggctgaccgcccaacgacccccgcccattgacgtcaataatgactgatgttcccatagtaacgccaatagggactttccattgacgctaatgggagtttgttttggcaccaaaatcaacgggactttccaaaatgtcgtaataaccccgccccgttgacgcaaatgggcggtaggcgtgtactctagaaggtctatataagcagagctcgtttagtgaaccgtttatactacttatctggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcacggtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcttgcggccggaattcacaaatggcagtattcatccacaattttaaaagaaaaggggggattggggggtacagtgcaggggaaagaatagtagacataatagcaacagacatacaaactaaagaattacaaaaacaaattactaaaaattcnaaaattttcgggtttattacagggacagcagagatccactttgggc

SEQ ID NO: 11 - DNA, 27 bp, Artificial Sequence
  DWV/VDV-1 (8004-8030), F
gtttgtatgaggttatacttcaaggag

SEQ ID NO: 12 - DNA, 24 bp, Artificial Sequence
  DWV/VDV-1 (8143-8120), R
gccatgcaatccttcagtaccagc

SEQ ID NO: 13 - DNA, 23 bp, Artificial Sequence
  VDV-1 CP (4890-4912), F
ctgtagttaagcggttattagaa

SEQ ID NO: 14 - DNA, 21 bp, Artificial Sequence
  VDV-1 CP (4986-4966), R
ggtgcttctggaacagcggaa

SEQ ID NO: 15 - DNA, 23 bp, Artificial Sequence
  DWV CP (4917-4939), F
ctgtagtcaagcggttacttgag

SEQ ID NO: 16 - DNA, 21 bp, Artificial Sequence
  DWV CP (5013-4993), R
ggagcttctggaacggcaggt

SEQ ID NO: 17 - DNA, 22 bp, Artificial Sequence
  VDV-1 NS (8623-8644), F
ttcattaaaaccgccaggctct

SEQ ID NO: 18 - DNA, 22 bp, Artificial Sequence
  VDV-1 NS (8723-8702), R
caagttcaggtctcatccctct

SEQ ID NO: 19 - DNA, 22 bp, Artificial Sequence
  DWV NS (8650-8671), F
ttcattaaagccacctggaaca

SEQ ID NO: 20 - DNA, 22 bp, Artificial Sequence
  DWV NS (8750-8729), R
caagttcgggacgcattccacg

SEQ ID NO: 21 - DNA, 21 bp, Artificial Sequence
  Varroa beta-actin, R
tgaaggtagtctcatggatac

SEQ ID NO: 22 - DNA, 22 bp, Artificial Sequence
  Varroa beta-actin, F
gtctctgttccagccctcgttc

SEQ ID NO: 23 - DNA, 23 bp, Artificial Sequence
  DWV/VDV-1 (4842-4864), F
cagtagcttgggcgattgtttcg

SEQ ID NO: 24 - DNA, 24 bp, Artificial Sequence
  DWV/VDV-1 (6747-6728), R
cgcgcttaacacacgcaaattatc

SEQ ID NO: 25 - DNA, 21 bp, Artificial Sequence
  DWV (6546-6526), R
cttggagcttgaggctctaca

SEQ ID NO: 26 - DNA, 20 bp, Artificial Sequence
  VDV-1 (6308-6289), R
ctgaagtactaatctctgag

SEQ ID NO: 27 - DNA, 20 bp, Artificial Sequence
  DWV/VDV-1 (9-28), F
gccttccatagcgaattacg

SEQ ID NO: 28 - DNA, 27 bp, Artificial Sequence
  DWV/VDV-1 (1092-1118), F
ttttcaatttaattttgatttcgaagg

SEQ ID NO: 29 - DNA, 18 bp, Artificial Sequence
  DWV/VDV-1 (1245-1228), R
cgccgcctagcttcatca

SEQ ID NO: 30 - DNA, 20 bp, Artificial Sequence
  DWV/VDV-1 (1990-2009), F
ggatgatccatttgataagg

SEQ ID NO: 31 - DNA, 23 bp, Artificial Sequence
  DWV/VDV-1 (2076-2054), R
catatagcatcagaattagcctc

SEQ ID NO: 32 - DNA, 19 bp, Artificial Sequence
  DWV/VDV-1 (3159-3177), F
gggtgcgtaaatatggtgg

SEQ ID NO: 33 - DNA, 20 bp, Artificial Sequence
  DWV/VDV-1 (3269-3250), R
tagtatctgaaacagcttcc

SEQ ID NO: 34 - DNA, 23 bp, Artificial Sequence
  DWV/VDV-1 (4614-4636), F
tgcctgagggccctattgcgaag

SEQ ID NO: 35 - DNA, 24 bp, Artificial Sequence
  DWV/VDV-1 (4716-4693), R
accatacccatatcttcacgcatc

SEQ ID NO: 36 - DNA, 24 bp, Artificial Sequence
  DWV/VDV-1 (6474-6497), F
cagagattgaagcgcatgaacaag

SEQ ID NO: 37 - DNA, 23 bp, Artificial Sequence
  DWV/VDV-1 (6750-6728), R
gcacttaacacacgcaaattatc

SEQ ID NO: 38 - DNA, 24 bp, Artificial Sequence
  DWV/VDV-1 (8028-8051), F
gagtatatacttatccataccatg

SEQ ID NO: 39 - DNA, 22 bp, Artificial Sequence
  DWV/VDV-1 (8141-8120), R
catgcaatccttcagtaccagc

SEQ ID NO: 40 - DNA, 23 bp, Artificial Sequence
  DWV/VDV-1 (10081-10066), R
gagtaacacctaacctgagtacc

SEQ ID NO: 41 - DNA, 25 bp, Artificial Sequence
  GB14044, F
gggtgtttacaccgcgatttattcg

SEQ ID NO: 42 - DNA, 32 bp, Artificial Sequence
  GB14044, R
gatattttttttctccacatttttattcgtcc

SEQ ID NO: 43 - DNA, 28 bp, Artificial Sequence
  GB10640, F
ctctatctcaagaccaacacctacttgc

SEQ ID NO: 44 - DNA, 27 bp, Artificial Sequence
  GB10640, R
ctgattgttgggcacgtccggcaacgc

SEQ ID NO: 45 - DNA, 28 bp, Artificial Sequence
  GB13140, F
cacaatctcaaaaatggatgttgatacg

SEQ ID NO: 46 - DNA, 31 bp, Artificial Sequence
  GB13140, R
aattaataactaattagcgaacgagcaatgg

SEQ ID NO: 47 - DNA, 29 bp, Artificial Sequence
  GB16716, F
ctgcaaaatggagccctctatggattgag

SEQ ID NO: 48 - DNA, 32 bp, Artificial Sequence
  GB16716, R
caaataatgcacctttggatctcttttagctg

SEQ ID NO: 49 - DNA, 23 bp, Artificial Sequence
  Adapter, F
cttggttagctgtgttgcagttg

SEQ ID NO: 50 - DNA, 46 bp, Artificial Sequence
  Adapter-VDV-1 CP (4890-4912), F
cttggttagctgtgttgcagttgctgtagttaagcggttattagaa

SEQ ID NO: 51 - DNA, 46 bp, Artificial Sequence
  Adapter-DWV CP (4917-4939), F
cttggttagctgtgttgcagttgctgtagtcaagcggttacttgag

SEQ ID NO: 52 - DNA, 10167 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggcgttttatagagtagattgccatgtgaccgctcatagaagtccattatggtctatcaatcgaagttgaatgtatttataaggacattatacttaattagtaatattagtagtccgtaactattatcatcctttttcagtttgatgtgataatagaccactgcagtatcgagtagagtttcggatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattattaaaaatggcctttagttgtggaactctttcttactctgccgttgcccaagctccgtctgtcgcctatgcacctcgtacatgggaagttgatgaagctaggcggcgccgagtcattaaacgtttggcgctggagcaagaacgtattcgtaacgttcttgacgttgatgtctatgcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatacggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattgtttgtgcctgctaaccagcaaggacccggcaaagtaagtaacggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaatgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctactgtagattttagtaataattgtagaggtagtaaccaggtatttgtatttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctggattagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagaaaatgctaaacaagcgttagagttagcatttgggtggggtcctgaatattttaattatgtcagaaataccattaaaatggcttttgacaaattgggtatttatgaggaccttatcacatgggaagaaatggatgttagatgttacgctagcgcgtaatattgaattttaaatattcattaattttaattttattttaggttattggaattgagggaagtaccaccccccaagaccttcgttttaaatctactaagaggagtaaacctatatataagagtctaaagacagagtggattagaccatcatctttagcttatatatggggaaggttgagttgcctctaaagactcagctccatagtagagtagttttaattacgattaaagtggtactctaagttaggtgttactcgcgtattatcaactagtggtaatgcgtcctaattttagtatagttttaaccataatagtaaaaaaaaaaaaaaaaaaaaaaaaaaa

SEQ ID NO: 53 - DNA, 10167 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggcgttttatagagtagattgccatgtgaccgctcatagaagtccattatggtctatcaatcgaagttgaatgtatttataaggacattatacttaattagtaatattagtagtccgtaactattatcatcctttttcagtttgatgtgataatagaccactgcagtatcgagtagagtttcgaatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattattaaaaatggcctttagttgtggaactctttcttactctgccgttgcccaagctccgtctgtcgcctatgcacctcgtacatgggaagttgatgaagctaggcggcgccgagtcattaaacgtttggcgctggagcaagaacgtattcgtaacgttcttgacgttgatgtctatgcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgcaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatacggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattgtttgtgcctgctaaccagcaaggacccggcaaagtaagtaacggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaatgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctactgtagattttagtaataattgtagaggtagtaaccaggtatttgtatttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctggattagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagaaaatgctaaacaagcgttagagttagcatttgggtggggtcctgaatattttaattatgtcagaaataccattaaaatggcttttgacaaattgggtatttatgaggaccttatcacatgggaagaaatggatgttagatgttacgctagcgcgtaatattgaattttaaatattcattaattttaattttattttaggttattggaattgagggaagtaccaccccccaagaccttcgttttaaatctactaagaggagtaaacctatatataagagtctaaagacagagtggattagaccatcatctttagcttatatatggggaaggttgagttgcctctaaagactcagctccatagtagagtagttttaattacgattaaagtggtactctaggttaggtgttactcgcgtattatcaactagtggtaatgcgtcctaattttagtatagttttaaccataatagtaaaaaaaaaaaaaaaaaaaaaaaaaaa

SEQ ID NO: 54 - DNA, 8856 bp, Deformed Wing Virus
gcccagacgacatgggaacaagaggacgcgcgggataatgagttcctaacggaacaattaaacaatttatatactatttattcgatcgctgaacgttgtacgcgtcggcccatcaaagagcactctcctatatcagttttgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgcaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatacggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattgtttgtgcctgctaaccagcaaggacccggcaaagtaagtaacggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaatgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctactgtagattttagtaataattgtagaggtagtaaccaggtatttgtatttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctggattagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagaaaatgctaaacaagcgttagagttagcatttgggtggggtcctgaatattttaattatgtcagaaataccattaaaatggcttttgacaaattgggtatttatgaggaccttatcacatgggaagaaatggatgttagatgttacgctagcgcgtaatattgaattttaaatattcattaattttaattttattttaggttattggaattgagggaagtaccaccccccaagaccttcgttttaaatctactaagaggagtaaacctatatataagagtctaaagacagagtggattagaccatcatctttagcttatatatggggaaggttgagttgcctctaaagactcagctccatagtagagtagttttaattacgattaaagtggtactctaggttaggtgttactcgcgtattatcaactagtggtaatgcgtcctaattttagtatagttttaaccataatagtaaaaaaaaaaaaaaaaaaaaaaaaaaaa

SEQ ID NO: 55 - DNA, 9250 bp, Deformed Wing Virus
cgttaatgtctcatagcccagacgatggcggatggaaagacatcactttttattttaatgctacgattattgctgttttattttgctgtttttatttgctattatattttgctattttcattattgctaaatatatttctttgctatttttgttttatatattagattcaattctttttattttatattttcaatttgattttgattttgaaggtaaatatatataaaaatggcatttagttgtggaactctttcttatgctgctgttgcccaagctccctctgtagctcatgctccccgtagttgggagattgatgaagctaggcgtcgacgcgttatcaagcgtttggcgttggaacaggaacggattcgaaacgttcttgacgtcactgtgtatgatcatacaacgtgggagcaagaggatgcgcgtgataatgagtttcttacggaacaattgaataatttgtatacgatatattctatagctgaaagatgtacccgccgccctgttcaagaacatgtccccatttcaatcagtaatagatattcccctctagaatcccttaagattgaggtaggagaagacgcgggtgagttcgtatttaagaaacccaaatatacaaagatttgtaagaaagtgaaacgggtggcatcaaaatttgtgcgcgagaaagttgttaggcccgtctgtaatcgatcgcccatgttgttatttaaaattaagaaagtaatatatgatttacatttgtatcggttacggaaacaagttcggcttctcagacgcgaaaaacagcgtgaatacgagttagagtgtgttactagtttgctacagctatctaattctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaacgatgttgtggatgattatgccactataacttcgcgttggtatcagattgccgagtttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtaattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcattttgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagtaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatacaatattggaataccaacttataatgctgaacgagcaagacaacttgctcagcatttgtatggtggtgggtctttgacagatgaaaaggctaagcaattgtttgtgcctgctaaccagcaaggacccggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaatgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctactgtagattttagtaataattgtagaggtagtaaccaggtatttgtatttttcaagaatacatttgaagtgttgaagaaaatgtggggttatgtgttttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcttatggacaaattttgctacatgatttaactgctgaaatgaatcaatcacgaaatttgagtgtgtttacacgtgtatatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagcgcgtatgtgcatgaacgttcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcaggatgttatctcttgcgtggaatgcgtcccgaacttgagattcaattatcaacgacacagttaatgaggaaaaaaggaataaaacctcacactatattcacggattgtttgaaagatacctgtttgcctgtggaaaaatgtagaatacctggtaagactagaatatttagtataagtccggtacagtttactataccctttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagagaatgctaagcaagcgctagagttagcattcggaggggtccagaatactttaactatgtcagaaatactattaaaatggcttttgacaagttgggtatttatgaagaccttatcacatgggaagaaatggatgttagatgttatgctagcgcgtagtatttaattttgaatacttattagttttaattttattttaggttattggaattgagggaagtaccaccccccaagaccttcgttttaaatctactaaaaggagtgaacctatatataagagtctaacgacagagtggatcagaccaccatctttagcttatatatgggaaaggttgagttgcctctaaagactcagctccatagtagagtagttttaattacgattaaagtggtactctaggttaggtgttactcgcgtattatcaactagtggtaatgcgtcctaattttagtataggtttaaccataattaagtaaaaaaaaaaaaaaaaaaaaaaaa

SEQ ID NO: 56 - DNA, 10144 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggtgttttatagagtagattgccatgtgaccgctcatagaagtccattatggtctatcaatcgaagttgaatgtatttataaggacattatacttaattagtaatattagtagtccgtaactattatcatcctttttcagtttgatgtgataatagaccactgcagtatcgagtagagtttcgaatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattactaaaaatggcctttagttgtggaactctttcttactctgccgttgcccaagctccgtctgtcgcctatgcacctcgtacatgggaagttgatgaagctaggcggcgccgagtcattaaacgtttggcgctggagcaagaacgtattcgtaacgttcttgacgttgatgtctatgcccagacgacatgggaacaggaggacgcgcgggataatgagttcctaacggaacaattgaacaatttatatactatttattcgattgctgaacgttgtacgcgtcggcccatcaaagagcactctcctatatcagtttcgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacctggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcatttcgtccctactggtatgcatagtttagctttaggtactaatctagtagagcctttgcatgcattacgattagatgcatcaggtacaacacaacatccagttgggtgtgcgcctgatgaggatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagcaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtgctatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacctggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttctttggtgaagcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgtaaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaatcaagtatttgtgtttttcaagaatacgtttgaagtgttgaagaaaatgtggggttatgtattttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtatatcgagagagtgtttgcggcacattcatatggacaaattttgctacatgatttaactgctgaaatgaatcaatcacgaaatttgagtgtgttcacacgtgtgtatgatcaaatttcaaaattgaagaccgatcttatggaaatgggatcgaatccatatataaggcgtgaatgttttacgatatgcatgtgtggtgcatctggaattggaaaatcatatttgaccgattctttatgcagcgagctcttacgtgcgagtcgtactcctgtgacaacaggcataaaatgtgttgttaatccattatccgattattgggatcaatgtgattttcagcctgttttgtgcgttgacgatatgtggagtgttgaaacatctactacgctcgataagcaactgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaagatgttgaaagattttcatcatattaaatttaggtatgcacatgacgtatgtaattccgagaccacatggtctgaatggatgacgtataatgaatttcttgaatggataactcctgtgtatatggctaaccgtcgtaaggcgaatgaatcgtttaagatgcgtgtggatgaaatgcaaatgttacgtatggatgaaccattggaaggtgataatattctcaataagtatgttgaagttaatcagcgcttagtggaggaaatgaaggcatttaaggagcgtacactatggtcagatttacatcgcgtaggtgcggaaattagtgcgtcagttaagaaagctttgccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagtgcgtatgcgcatgaacgctcatcttcaactcggctttctacggatgacgtcaagttatataaaacaattagtatgttacatcaaaagtatgataccacagagtgtgctaaatgtcaacattggtatgctccgttgactgatatctatgttgatgacaagaaattgttctggtgtcagaaagagaaaaagacacttattgatgtccgcaaattgtcgaaagaagatgtgactgttcaatcaaaattgattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattggctgatcattatgtgaataggcattgtagctctgatttttggtcaccaggactggcatcacctcaaggattgaaatatagtgaagcagtaacagtaaaggcacctagaattcatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacgcagttaatgaggaaaaagggaataaaacctcacactatattcacagattgtttgaaagatacctgtttgcctgtggaaaaatgtagaatacctggtaagactagaatatttagtataagtccggtacagtttactataccctttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagatgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtatcggtagaaggaacgacaaattggacccatgctcgaggattgggtcgtcgtacagcaaccatagagaatgctaaacaagcgctagagttagcattcggatggggtccagaatactttaaccatgttagaagcaccattaaaatggcttttgacaagttgggtatttatgaagaccttatcacatgggaagaaatggatgttagatgttatgctagcgcgtagtatttaattttgaatactcattagttttaattttattttaggttattggaattgagggaagtaccaccccccaagaccttcgttttaaatctactaagaggagtgaacctatatataagagtctaaagacagagtggattagaccatcatctttagcttatatatggggaaggttgagttgcctctaaagactcagctccgtagtagggtagttttaattacgattaaagtggtactctaggttaggtgttactcgcgtattatcaattagtggtaatgcgtcctaattttagtatagttttaaccataatagtacgt

SEQ ID NO: 57 - DNA, 10139 bp, Deformed Wing Virus
cgatttatgccttccatagtgaattacggtgcaactaacaattttagatagtagccatgaacaaacattatgattactcactacgtattgatcatttttataatgacttgcgtagcatgaagcgcatgcttgtagttgtaactatgttattttgcaagttggagttaattgtattggattatgagtgcgtgcactcagtgtctgtatttatagtcgtttgtggttcaaggttttgtgttagtagtacaatcttgtatgaatgtacctctagtatgaatgatattgaatgacagtatcgaaggataaatctttataaaatacaaaaatattgtttttattatttcgatacggtgttttatagagtagattgccatgtgaccgctcatagaagtccattatggtctatcaatcgaagttgaatgtatttataaggacattatacttaattagtaatattagtagtccgtaactatcataatcctattttagtttgatgtgataatagaccactgcagcagctactagagtttagaaagagtagtgcaatagtatgatcactgtcaccgaccatctattgtaatgtgaggtttgtcggaaaccagtatttttgaagtgactagcaatcatggattaaattagatggtattctagtttagaggtgattcggcgctgcggtgcgactgaaacttctaaattagcatgtcaggttatattatgaatgcgttagtagtaatttctgcgatagagccgggacccctctatctctcaggtattgtatgaggcgaaagtgtgaaagttttgtatgtctctatacataagtgactgtatcgggatttcctttggcaagaatccttttaatacagtataatttatgctacggtacgttacgttcgcagtgcacccgttaatgtcacatagcccagacgatggcgaatggaaagacattattttttattttaatgctgtatttattgctgatttattttgctgtttttatttgctattatatatttgctaattttcattattgcgaaatatatttctttgctatttttattatatatgttagattcaattctattttttctatattttcaatttaattttgatttcgaaggtaaatatatataattaattactaaaaatggcatttagttgtggaactctttcttatgctgctgttgcccaagctccgtctgtagcccatgctccccgtagttgggagattgatgaagctaggcggcgccgagttattaaacgtttggcgctggagcaagaacgtattcgtaacgttcttgacgttgatgtctatgcccagacgacatgggaacaggaggacgcgcgggataatgagttccttacggaacaattgaataatttatatactatttattcgattgctgaacgttgtacgcgtcggcccatcaaagagcactctcctatatcagtttcgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatatgagttagagtgtgttactagtttgctacagctatctaatcctgtttcagctaaacctgagacggacaatcctaatcctggtccagatggcgaaggtgaagttgaattagaaaaggatagtaatgtagtattaactacacaacgtgatccgagtacttctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattgcaggcaacttggtactattcggatcatgaaaatttgaatatccagacgaagcgaagtgtgtatggtttttcgcatatggatcatgctttgattagcgcatcagcgagtaatgaagcaaaattaatgataccttttaaacatgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttagcagaaggattactaaataatactgtaggtggttgtaatatggataacccgtcatatcagcaatctccgcgtcattttgtccctactggtatgcatagtttagctttaggcactaatttagtagagcctttgcatgcattacgattagatgcatcaggtacaacacaacatccagttgggtgtgcgcctgatgaagatatgactgtatcttccattgcatcacgatacggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatacagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagcaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacccggcaaagtaagtaacggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaggcatttaacgatcttaagacgttaatgcgccgataccaattatatggtcaattattgttatccgttactacggataaggatattgatcattgtatgtttaccttcccttgtttacctcaagggctagcgttagatataggttcggctggatctcctcatgaaatatttaatcgctgccgtgatggtatcattccattgatagcgtcagggtatcggttttatcgaggcgatttacggttcaaaattgttttcccaagtaacgttaatagcaatatttgggtacaacaccgaccagatcgtagactgaaaggatggtctgaagcgaaaatagtaaactgtgatgctgtatctactggacaaggcgtttataatcatggatatgctagtcatattcagatgacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgatattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagtccaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaaccaggtatttgtatttttcaagaatacatttgaagtgttgaagaaaatgtggggttatgtattttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcatatggacaaattttgctacatgatttaactgctgaaatgaatcaatcacgaaatttgagtgtgtttacacgtgtgtatgatcaaatttcaaaattgaaaaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgggattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgcagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatattctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacaaaagaaatatatagctacgtggtgtagtaagcgtcgtgaacatattgctgactttgatcttgtgtggactgataatttgcgtgtgttaagcgcgtatgggcatgaacgttcatcttcaactcggctttctacggacgacgttaagttatataaaacgattagcatgttacatcaaaagtatgataccacagagtgtgctaaatgtcaacattggtatgctccgttgactgatatttatgttgatgacaaaaaattgttctggtgccagaaagagaaaaagacacttattgatgtccgaaaattgtcgaaagaagatgtgactgttcaatcaaaattaattaatttatctgttccttgtggtgaagtgtgtatgttacattcaaaatatttcaattatcttttccataaagcatggttgtttgagaacccaacttggcgcctaatatataatggtaccaagaagggtatgcccgagtactttatgaattgtgtggatgaaatttcattagattctaaatttggtaaagtgaaagtgtggttgcaagcgatcattgataagtatttaactcgtcccgtgaaaatgattcgtgattttcttttcaagtggtggccgcaagttgcgtatgtgttgagcttgctaggtataatcggtataactgcgtatgaaatgagaaatccgaaaccaacgtctgaggaattagctgatcattatgtgaataggcattgtagctctgatttttggtcaccaggactggcatcaccccaaggattgaaatatagtgaagcagtaacagtaaaggcacctagaatccatagattgccagttactactaagcctcagggatcaacacaacaagtagacgctgctgtgaataaaattttacagaatatggtttacattggtgttgttttcccgaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacctagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttacccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttgtatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaacaggaaacatctggaattagcgacaaatcatttgaaagaaaaattagtttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggccctggattagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacaacgaattggacccatgctcgaggattgggtcgtcgtacagcaaccatagaaaatgctaaacaagcgttagagttagcatttgggtggggtcctgaatactttaattatgtcagaaataccattaaaatggcttttgacaaattgggtatttatgaagaccttatcacatgggaagaaatggatgttagatgttacgctagcgcgtaatattgaattttgaatattcattaattttaattttattttaggttattggaattgagggaagtaccaccccccaagaccttcgttgtaaatctactaagaggagtgaacctatatataagagtctaaagacagagtggattagaccatcatctttagcttatatatggggaaggttgagttgcctctaaagactcagctccatagtagagtagttttaattacgattaaagtggtactctaagttaggtgttactcgcgtattatcaattagtgggaatgcgtcctaattttagtatagttttaaccataatag

SEQ ID NO: 58 - DNA, 10140 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggcgttttatagagtagattgccatgtgaccgctcatagaagtccattatggtctatcaatcgaagttgaatgtatttataaggacattatacttaattagtaatattagtagtccgtaactattatcatcctttttcagtttgatgtgataataaaccactgcagtatcgagtagagtttcggatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattattaaaaatggcctttagttgtggaactctttcttactctgccgttgcccaagctccgtctgtcgcctatgctccccgtacatgggaagttgatgaagctaggcggcgccgagtcattaaacgtttggcgctggagcaagaacgtattcgtaacgttcttgacgttgatgtctatgcccagacgacatgggaacaggaggacgcgcgggataatgagttcctaacggaacaattgaacaatttatatactatttattcgattgctgaacgttgtacgcgtcggcccatcaaagagcactctcctatatcagtttcgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacttggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcatttcgtccctactggtatgcatagtttagctttaggcactaatctagtagagcctttgcatgcattacgattagatgcatcaggtacaacacaacatccagttgggtgtgcgcctgatgaagatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagagattctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagcaattcgcttgtttttagatggggttcggcttcagatcaaattgctcaatggccaacaataacagtgcctcgagaagagttggcattcttgcgtatccgcgatgctaagcaagctgctgtaggaacgcaaccttggcgtactatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacctggcaaagtaagtaacggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttcttcggtgaagcatttaacgatcttaagacgttaatgcgccgataccaattatatggtcaattattgttatccgttactacggataaggatattgatcattgtatgtttaccttcccttgtttacctcaagggttagcgttagatataggttcggctgggtctcctcatgaaatattcaatcgctgccgtgatggtatcattccactgatagcgtcagggtatcggttttatcgaggcgatttacggttcaaaattgttttcccaagtaacgttaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctactgtagattttagtaataattgtagaggtagtaaccaggtatttgtatttttcaagaatacatttgaagtgttgaagaaaatgtggggttatgtattttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtacatcgagagagtgtttgcggcacattcatatggacaaattttgctacatgatttaactgctgaaatgaatcaatcacgaaatttgagtgtgtttacacgtgtgtatgatcaaatttcaaaattgaagaccgatcttatggaaatgggatcgaatccatatataaggcgtgaatgttttacgatatgtatgtgtggtgcatctggaattggaaaatcatatttgaccgattctttatgcagcgagctcttacgtgcgagtcgtactcctgtgacaacaggcataaaatgtgttgttaatccattatctgattattgggatcaatgtgattttcagcctgttttgtgcgttgacgatatgtggagtgttgaaacatctactacgctcgataagcagttgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaaaatgttgaaagattttcatcatattaaatttaggtatgcacatgatgtgtgtaattccgaaaccacatggtctgaatggatgacgtataatgaatttcttgaatggataactcctgtgtatatggctaaccgtcgtaaggcgaatgaatcgtttaagatgcgtgtggatgaaatgcaaatgctacgtatggatgagccattggaaggtgataatatcctcaataagtatgttgaagttaatcagcgcttagtggaggaaatgaaggcatttaaggagcgtacactatggtcagatttacatcgcgtaggtgcggaaattagtgcgtcagttaagaaagctttaccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagtgcgtatgcgcatgaacgctcatcttcaactcggctttctacggacgacgttaagttatataaaacgattagcatgttacatcaaaagtatgataccacagagtgtgctaaatgtcaacattggtatgctccgttgactgatatctatgttgatgacaaaaaattgttctggtgtcagaaagagaaaaagacacttattgatgtccgaaaattgtcgaaagaagatgtgactgttcaatcaaaattgattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaatccatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagctgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgtggaaaaagggaataaaacctcacactatattcacggattgtttgaaagatacctgtttgcctgtggaaaaatgtagaatacctggtaagactagaatatttagtataagtccggtacagtttactataccttttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctggattagattccgatgttgcagcttcagcgtttgaaattattatcgactgggtattacattatactgaagaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttttcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtatcggtagaaggaacgacgaattggacccatgctcgaggattgggtcgtcgtacagcaaccatagaaaatgctaaacaagcgttagagttagcattcgggtggggtcctgaatattttaaccatgtcagaaataccattaaaatggcttttgacaaattgggtatttatgaagaccttatcacatgggaagaaatggatgttagatgttacgctagcgcgtaatattgaattttaaatattcattaattttaattttattttaggttattggaatcgagggaagtaccaccccccaagaccttcgttttaaatctactaagaggagtgaacctatatataagagtctaaagacagagtggattagaccatcatctttagcttatatatggggaaggttgagttgcctctaaagactcagctccatagtagagtagttttaattacgattaaagtggtactctaggttaggtgttactcgcgtattatcaactagtggtaatgcgtcctaattttagtatagttttaaccataatagt

SEQ ID NO: 59 - DNA, 10166 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggtgttttatagagtagattgccatgtgaccgctcatagaagtccattatggtctatcaatcgaagttgaatgtatttataaggacattatacttaattagtaatattagtagtccgtaactattatcatcctttttcagtttgatgtgataatagaccactgcagtatcgagtagagtttcgaatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctctgtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattactaaaaatggcctttagttgtggaactctttcttactctgccgttgcccaagctccgtctgtcgcctatgcacctcgtacatgggaagttgatgaagctaggcggcgccgagtcattaaacgtttggcgctggagcaagaacgtattcgtaacgttcttgacgttgatgtctatgcccagacgacatgggaacaggaggacgcgcgggataatgagttcctaacggaacaattgaacaatttatatactatttattcgattgctgaacgttgtacgcgtcggcccatcaaagagcactctcctatatcagtttcgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacctggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcatttcgtccctactggtatgcatagtttagctttaggtactaatctagtagagcctttgcatgcattacgattagatgcatcaggtacaacacaacatccagttgggtgtgcgcctgatgaggatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagcaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtgctatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacctggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttctttggtgaagcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgtaaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaatcaagtatttgtgtttttcaagaatacatttgaagtgttgaagaaaatgtggggttatgtattttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtatatcgagagagtgtttgcggcacattcatatggacaaattttgctacatgatttaactgctgaaatgaatcaatcacgaaatttgagtgtgttcacacgtgtgtatgatcaaatttcaaaattgaagaccgatcttatggaaatgggatcgaatccatatataaggcgtgaatgttttacgatatgcatgtgtggtgcatctggaattggaaaatcatatttgaccgattctttatgcagcgagctcttacgtgcgagtcgtactcctgtgacaacaggcataaaatgtgttgttaatccattatccgattattgggatcaatgtgattttcagcctgttttgtgcgttgacgatatgtggagtgttgaaacatctactacgctcgataagcaactgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaagatgttgaaagattttcatcatattaaatttaggtatgcacatgacgtatgtaattccgagaccacatggtctgaatggatgacgtataatgaatttcttgaatggataactcctgtgtatatggctaaccgtcgtaaggcgaatgaatcgtttaagatgcgtgtggatgaaatgcaaatgttacgtatggatgaaccattggaaggtgataatattctcaataagtatgttgaagttaatcagcgcttagtggaggaaatgaaggcatttaaggagcgtacactatggtcagatttacatcgcgtaggtgcggaaattagtgcgtcagttaagaaagctttgccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagtgcgtatgcgcatgaacgttcatcttcaactcgactatctacggatgacgtcaggttatataaaacgattagcatgttacatcaaaagtatgataccacagagtgtgctaaatgtcaacattggtatgctccgttgactgatatttatgttgatgacaagaaattgttctggtgtcagaaagagaaaaagacacttattgatgtccgcaaattgtcgaaagaagatgtgactgttcaatcaaaattgattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgttaggtataattggtataactgcgtatgaaatgagaaatccgaaaccaacgtctgaggaattagctgatcattatgtgaataggcattgtagctctgatttttggtcaccaggactggcatcacctcaaggattgaaatatagtgaagcagtaacagtaaaggcacctagaattcatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattagtttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacggattgtttgaaagatacctgtttgcctgtggaaaaatgtagaatacctggtaagactagaatatttagtataagtccggtacagtttactataccctttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaggaagataataaagacgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtttcggtagaaggaacgacgaattggacccatgctcgaggattgggtcgtcgtacagcaaccatagagaatgctaaacaagcgctagagttagcattcggatggggtccagaatactttaactatgttagaagcaccattaaaatggcttttgacaagttgggtatttatgaagaccttatcacatgggaagaaatggatgttagatgttatgctagcgcgtagtatttaattttgaatactcgttagttttaattttattttaggttattggaattgagggaagtaccaccccccaagaccttcgttttaaatctactaagaggagtgaacctatatataagagtctaaagacagagtggattagaccatcatctttagcttatatatggggaaggttgagttgcctctaaagactcagctccgtagtagggtagttttaattacgattaaagtggtactctaggttaggtgttactcgcgtattatcaattagtggtaatgcgtcctaattttagtatagttttaaccataatagtaaaaaaaaaaaaaaaaaaaaaaaaaa

SEQ ID NO: 60 - DNA, 10141 bp, Deformed Wing Virus
cgatttatgccttccatagcgaattacggtgcaactaacaattttagatagtagccatgaacaaacattatagtggctcactacgtattgatcatttttataatgacttgcgtagcatgaagcgcatgcttgtagttgtaactatgttactttacaagttggagttcactatcttggattatgagtatgtgcacttagtgtctgtatttatagtcgtttgtggttcaaggttttgtgttagtagtacacttatgtatgaatgtacctctagtatgaatgttatagaatgacagtatcgaaggaaaaatctttataaaatacaaaaatattgtttttattatttcgatacggtgttttatagagtagattgccatgtgaccgctcatagaagtccattatggtctatcaatcgaagttgaatgtatttataaggacattatacttaattagtaatattagtagtccgtaactattatcatcctttttcagtttgatgtgataatagaccactgcagtatcgagtagagtttcgaatgcgtagtgcaatagtataatcactgtcaccgaccatctattgtaatgatagatctgtcggaaaccattatttatgaagtgactagcaatcatggattaaattagatggtattctagtttagaggtgattcggcgctgcggtgcgactgaaacttctaaattagcatgtcaggttatattatgaatgcgttagtagtaatttctgcgatagagctgggacccctcagtctctcaggtattgtatgaggcgaaagtgtgaaagttttgtatgtgtttttatatgtacgactgtatcgggaattcctttagcaagaatccttttaatacagtataatctgtgctacggtacgttacgttcgcagggcacccgttaatgtctcatagcccagacgatggcggatggaaagacatcatattttattttaatgctgtctttattgctgatttattttgctgtttttatttgctattttatatttgctaattttcattattgcgaaatatattacattgctatttttattatatacgctagattcaattttattttttctatattttcaatttaattttgatttcgaaggtaaatatatataattaattactaaaaatggcctttagttgtggaactctttcttactctgccgttgcccaagctccgtctgtcgcctatgcacctcgtacatgggaagttgatgaagctaggcggcgccgagtcattaaacgtttggcgctggagcaagaacgtattcgtaacgttcttgacgttgatgtctatgcccagacgacatgggaacaggaggacgcgcgggataatgagttcctaacggaacaattgaacaatttatatactatttattcgattgctgaacgttgtacgcgtcggcccatcaaagagcactctcctatatcagtttcgaataggtttgctccactggaatctctcaaggtcgaggtcggtcaagaagcaggcgaatgtatatttaagaaacctaaatatacgcgcgtttgcaagaaagtgaagcgcgttgcaactcgcttcgttcgtgaaaaagtcgttcgtcctatgtgttctagatcccctatgctcttatttaagcttaagaaaattatttatgatttgcacttatatagattaagaaaacagattaggcttctcagacgcgaaaaacagcgtgaatacgagttagagtgtgttactagtttgctacagctatctaatcctgtttcagctaaacctgagatggacaatcctaatcctggtccagatggtgaaggtgaagttgaattagaaaaggatagtaatgtagtattaactacacaacgtgatcctagtacctctattcctgctccaactagtgtgaagtggagtagatggactagtaatgatgttgtggatgattatgccactataacttcgcgttggtatcagattgccgaatttgtatggtcaaaggatgatccatttgataaggaattggcgcgtttaattttacctcgagctttgttatctagtattgaggctaattctgacgctatttgtgatgtacctaatactattccgtttaaggtacatgcatattggcgtggagatatggaagttcgagtgcagattaactcgaataaattccaggttggtcaattacaggcaacctggtactattcggatcatgaaaatttgaatatccagacgaagcgaagtgtgtatggtttttcgcatatggatcatgctttgattagtgcatcagcgagtaatgaagcaaaattagtgataccttttaaacacgtatatccattcttaccaacgcgtgtcgttcctgattggacaactggtattcttgatatgggtaccttaaatattcgtgtgattgctccactacgtatgagtgcgacgggaccaaccacttgtaatgttgtagtatttattaagttaaataatagtgaattcactggtacttcttctggtaagttttacgcgaatcaaatcagggcaaaacctgaaatggaccgtgtgttaaatttggcagaaggattactaaataatactgtaggtggttgtaatatggataacccgtcatatcagcaatctccgcgtcatttcgtccctactggtatgcatagtttagctttaggtactaatctagtagagcctttgcatgcattacgattagatgcatcaggtacaacacaacatccagttgggtgtgcgcctgatgaggatatgactgtatcttccattgcatcacgatatggtttaattcgccaagtgcaatggaagaaagaccatgcgaaaggatcattattattacaacttgacgctgatcctttcgttgaacagaaaattgagggaaccaatccaatttctttgtattggtttgctccggttggagtcgtatctagtatgtttatgcaatggagaggttctttagaatatagatttgacattatagcttcccaatttcatacgggtaggttaattgtaggttatgttcctggactgactgcttctttacaacgtcaaatggactatatgaaattgaaatcatctagttatgtggtgtttgatttacaggaaagtaatagttttacgtttgaagtgccctatgtgtcatatagaccgtggtgggtgcgtaagtatggtggtaattatctgccatcttctactgatgcgcctagcacactgtttatgtatgtacaagtaccattgatacctatggaagctgtttctgatactatagatatcaatgtgtatgtgcgtggtggcagttcgtttgaggtttgtgttccagtccaacctagtttaggtttgaactggaatacagatttcatattacgtaatgatgaggagtaccgcgcaaagaatggatatgcaccatattatgctggtgtgtggcatagcttcaataatagcaattcgcttgtttttagatggggttcggcttcagatcaaattgctcaatggccaacaataacagtgcctcgaggagagttggcattcttgcgtatccgcgatgctaagcaagctgctgtaggaacgcaaccttggcgtgctatggtcgtttggccttcaggtcatggatataatattggaataccaacttataatgctgaacgagcaagacaacttgctcagcatttgtatggtggtgggtctttgacagatgaaaaggctaagcaattatttgtgcctgctaaccagcaaggacctggcaaagtaagtaatggtaaccctgtctgggaagtaatgcgcgcgcctcttgcaactcagcaagcgcatatacaagattttgaatttgttgaagctgttccagaaggcgaagaatcacgcaacactacggtgctagatacgacaacaacgttacagtctagcggatttggtcgcgctttctttggtgaagcatttaacgatcttaagacgttaatgcgccgataccaattatatggtcaattattgttatccgttactacggataaggatattgatcattgtatgtttaccttcccttgtttacctcaagggttagcgttagatataggttcggctggatctcctcatgaaatattcaatcgctgccgtgatggtatcattccactgatagcgtcagggtatcggttttatcgaggcgatttacggttcaaaattgttttcccaagtaacgtaaatagcaatatttgggtacaacaccgaccagatcgtagactgaaaggatggtctgaagcgaaaatagtaaactgtgatgctgtatctactggacaaggcgtttataatcatggatatgctagtcatattcagattacgcgtgtaaataatgttatagaattggaagtcccgttttataacgctacgtgctataattatttgcaagcgtttaacccatctagtgcagcgtcgagttatgccgtttcgctcggagagatttcggttggttttcaagctactagtgatgacattgcagccatagttaataaacctgtaactatatattacagtattggcgatggtatgcagttttcgcagtgggttggttatcaaccaatgatgattctagatcaattgccagcaccagtagttagggcagtgcctgagggccctatagcgaagataaagaactttttccaccaaacggcagatgaagttcgagaagctcaggccgcaaagatgcgtgaagatatgggtatagtagttcaagacgttataggagagttaagtcaggctatacccgatcttcaacaaccggaagttcaagcgaatgttttttctctggtgtcacagttagtgcatgctatcatcggtactagtcttaagacagttgcttgggcgattgtttcgatttttgtaactttaggtttgattggacgtgaaatgatgcattcagtcataactgtagttaagcggttattagaaaaatatcacttggcgacgcaaccccaggaatccgccaattcaggtacggttatttccgctgttccagaagcacctaatgctgaagcagaggaggctagtgcctgggtatccattatttataatggtgtgtgtaatatgttgaatgtagccgctcaaaaaccgaaacaatttaaagattgggtaaaattagctaccgtagattttagtaataattgtagaggtagtaatcaagtatttgtgtttttcaagaatacgtttgaagtgttgaagaaaatgtggggttatgtattttgtcagagtaatcctgcagcgcgtttgttgaaagctgtgaatgacgagcctgagattttgaaagcatgggtgaaggaatgtctgtatttggatgatcccaaattcagaatgcgtcgagcgcatgatcaagagtatatcgagagagtgtttgcggcacattcatatggacaaattttgctacatgatttaactgctgaaatgaatcaatcacgaaatttgagtgtgttcacacgtgtgtatgatcaaatttcaaaattgaagaccgatcttatggaaatgggatcgaatccatatataaggcgtgaatgttttacgatatgcatgtgtggtgcatctggaattggaaaatcatatttgaccgattctttatgcagcgagctcttacgtgcgagtcgtactcctgtgacaacaggcataaaatgtgttgttaatccattatccgattattgggatcaatgtgattttcagcctgttttgtgcgttgacgatatgtggagtgttgaaacatctactacgctcgataagcaactgaatatgcttttccaggtccattcccctatcgtgctttcccctcctaaagctgatttagaaggtaagaaaatgcgatataacccggaaatattcatatacaatacgaataaacctttcccgaggtttgatcgtattgctatggaagctatttatcggcgtagaaatgttttgattgaatgtaaagcgagtgaagagaagaagcgaggatgtaagcattgtgagaatgatatccctattgctgaatgtagtcctaagatgttgaaagattttcatcatattaaatttaggtatgcacatgacgtatgtaattccgagaccacatggtctgaatggatgacgtataatgaatttcttgaatggataactcctgtgtatatggctaaccgtcgtaaggcgaatgaatcgtttaagatgcgtgtggatgaaatgcaaatgttacgtatggatgaaccattggaaggtgataatattctcaataagtatgttgaagttaatcagcgcttagtggaggaaatgaaggcatttaaggagcgtacactatggtcagatttacatcgcgtaggtgcggaaattagtgcgtcagttaagaaagctttgccaaccatttccataaccgaaaaattaccacattggactgttcaatgtggtattgctaaacctgagatggaccatgcttatgaggttatgagttcgtatgcagctggaatgaatgcagagattgaagcgcatgaacaagttcggcgttcatcagtggaatgtcaatatgcagagcctcaagctccaagaaatcctgatgatgaaggaccaaccatagatgaagaacttatgggcgacactgaattcacatcacaggctctagaacgtcttgtggatgaaggttatataactggaaaacagaagaaatatatagctacgtggtgtagtaagcgtcgtgaacatactgctgactttgatcttgtgtggactgataatttgcgtgtgttaagtgcgtatgcgcatgaacgctcatcttcaactcggctttctacggatgacgtcaagttatataaaacaattagtatgttacatcaaaagtatgataccacagagtgtgctaaatgtcaacattggtatgctccgttgactgatatctatgttgatgacaagaaattgttctggtgtcagaaagagaaaaagacacttattgatgtccgcaaattgtcgaaagaagatgtgactgttcaatcaaaattgattaatttatctgttccttgtggtgaagtgtgtatgttacattcaaaatatttcaattatcttttccataaagcatggttgtttgagaacccaacttggcgcctaatatataatggtaccaagaagggtatgcctgagtactttatgaattgtgtggatgaaatttcattagattccaaattcggtaaagtgaaagtatggttgcaagcgatcattgataagtatttaactcgtcccgtgaaaatgattcgtgattttcttttcaagtggtggccgcaagttgcgtatgtgttgagcttgctaggtataattggtataactgcgtatgaaatgagaaatccgaaaccaacgtctgaggaattggctgatcattatgtgaataggcattgtagctctgatttttggtcaccaggactggcatcacctcaaggattgaaatatagtgaagcagtaacagtaaaggcacctagaattcatagattgccagttactactaagcctcagggatcaacacaacaagtagacgctgctgtgaataaaattctacagaatatggtttatattggtgttgttttcccaaaagtgcctggtagtaagtggcgagatattaattttaggtgtcttatgcttcataataggcaatgtttaatgttgaggcattatattgagtcaacagccgcctttcctgagggaaccaagtactattttaagtatattcataatcaagagactagaatgtctggtgatatttctggtattgaaattgatttgttgaatttacccagattgtattatggtggtcttgcgggagaggagtcatttgatagcaatatcgtgcttgtgactatgcctaatcgtattcctgagtgtaagagcattgttaaatttatagcgtcacataatgaacatatacgtgctcagaatgatggagtgttagtaactggcgaccatactcagctgttggctttcgagaataataataagactccaataagtatcaacgctgatggtttgtatgaggttatacttcaaggagtatatacttatccataccatggcgacggtgtttgtggttcgatattgctgtctcggaatttacaacggccgattataggtatccatgttgctggtactgaaggattgcatggctttggagttgctgaaccactggtacatgaaatgttcactggtaaagcaatcgagagtgaaagagagccgtatgatcgtgtgtatgaacttccattgcgtgaattagatgaatccgatattggtttagataccgacttatatccgattggtagagtggatgcaaagttagctcatgctcaaagcccttctactgggatcaaaaagacgcttatccatggaacatttgatgtaaggactgaaccaaatccgatgtcgtcacgtgatccaagaatagcgccgcatgatcctttgaagttagggtgtgaaaagcatggtatgccttgttcaccgtttaataggaaacatctggaattagcgacaaatcatttgaaagaaaaattaatttcagtagttaaaccaataaatggttgcaagattagaagtttgcaagatgctgtatgtggtgtgcctggtgtagatgggtttgattcgatatcttggaatactagtgctggttttcctttgtcttcattaaagccacctggaacatcaggtaagcgatggttgtttgacattgagctacaagactcgggatgttatctcttgcgtggaatgcgtcccgaacttgagattcaattatcaacgacacagttaatgaggaaaaagggaataaaacctcacactatattcacagattgtttgaaagatacctgtttgcctgtggaaaaatgtagaatacctggtaagactagaatatttagtataagtccggtacagtttactataccctttagacagtattacttagattttatggcatcctatcgagctgcacgacttaatgctgagcatggtattggtattgatgttaacagcttagaatggacaaatttggcaacaagtctgtcaaagtatggcactcatatcgtgacgggtgactataagaattttggtcctgggttagattccgatgttgcagcttcagcgtttgaaattattatcgactgggtattacattatactgaagaagataataaagatgaaatgaagcgagtaatgtggaccatggcgcaggagattttagcgcctagtcatctatgtcgtgatttagtgtaccgagtaccttgtggaattccatcaggttctccgataacggacattttgaatacaatttcgaattgtctgttgattaggttagcttggttaggtattactgatttgcctttatccgagttctctcaaaatgttgttcttgtttgttatggtgatgatcttatcatgaatgttagtgataacatgattgataaatttaatgctgtgacaatagggaaattcttttcacaatataagatggaatttacggatcaggacaaatcaggaaatactgtgaagtggcggacgttacagactgctactttcttgaagcatgggtttttaaaacatccaactagacctgtgtttctggctaacctagacaaggtatcggtagaaggaacgacaaattggacccatgctcgaggattgggtcgtcgtacagcaaccatagagaatgctaaacaagcgctagagttagcattcggatggggtccagaatactttaaccatgttagaagcaccattaaaatggcttttgacaagttgggtatttatgaagaccttatcacatgggaagaaatggatgttagatgttatgctagcgcgtagtatttaattttgaatactcattagttttaattttattttaggttattggaattgagggaagtaccaccccccaagaccttcgttttaaatctactaagaggagtgaacctatatataagagtctaaagacagagtggattagaccatcatctttagcttatatatggggaaggttgagttgcctctaaagactcagctccgtagtagggtagttttaattacgattaaagtggtactctaggttaggtgttactcgcgtattatcaattagtggtaatgcgtcctaattttagtatagttttaaccataatagaa

SEQ ID NO: 61 - DNA, 10141 bp, Deformed Wing Virus; identical to SEQ ID NO: 60

SEQ ID NO: 62 - DNA, 10141 bp, Deformed Wing Virus; identical to SEQ ID NO: 60

SEQ ID NO: 63 - DNA, 20 bp, Artificial Sequence
  Honeybee beta-actin, F
aggaatggaagcttgcggta

SEQ ID NO: 64 - DNA, 21 bp, Artificial Sequence
  Honeybee beta-actin, R
aattttcatggtggatggtgc

SEQ ID NO: 65 - DNA, 18 bp, Artificial Sequence
  AFB-P. larvae, F
cgggagacgccaggttag

SEQ ID NO: 66 - DNA, 20 bp, Artificial Sequence
  AFB-P. larvae, R
ttcttccttggcaacagagc

SEQ ID NO: 67 - DNA, 24 bp, Artificial Sequence
  EFB-M. plutonius, F qPCR
tgttgttagagaagaataggggaa

SEQ ID NO: 68 - DNA, 18 bp, Artificial Sequence
  EFB-M. plutonius, R qPCR
cgtggctttctggttaga

SEQ ID NO: 69 - DNA, 29 bp, Artificial Sequence
  Honeybee Rp49 (GB10903), F
caaaaaaactcgtcatatgttgccaactg

SEQ ID NO: 70 - DNA, 26 bp, Artificial Sequence
  Honeybee Rp49 (GB10903), R
gcatcattaaaacttccagttccttg

SEQ ID NO: 71 - DNA, 22 bp, Artificial Sequence
  Honeybee Vago (GB10896), F
gtcatagcgatcgttttcgctg

SEQ ID NO: 72 - DNA, 41 bp, Artificial Sequence
  Honeybee Vago (GB10896), R
gctataatacgactcactatagggcaattagggaatgcagc

SEQ ID NO: 73 - DNA, 11100 bp, Artificial Sequence
  pTYF-mCMW/SYN-EGFP (2)
ggcctagggataacagggtaatgatatctctgaccgctagcatcgtcgacccgggatccgcggactagtcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcagtagtttaacacattatacacttaaaaattggatctacttattcaagatcggccgctggagctgatctgactcagcagagcagagaagtccatgtccgcaatggaggagaagtcttcatctccggagagaccattgggaagccccgaggtccccaggggtgtgggagctgggtcagggggcctctgggaccctgtcaccaggcgagttatagcttcagggtactccatcagcatgggctcagctgtggagtgagacatggacacaccctggttcaggagctgctgaaactctgagttgtccacagatgccaggtctgtgaacactcctgggtctgtgctgttgccaagcaaggcccccaagtcttcatcagcatcaaactgcaggtgcagcagggcttccgacagcgtgccttccccagcctgggtgctctttggaacaggtgcagacagggactggggaggacccggggttagaactggggcaggagctgggggctgagccagaggtaccatggctgaggaagggaccatggtctgggcaaggactggggcagaggacggtgctaaggccagggcctggtttgagatctgcccagaaggaaacaccatggtgagctctgcagaagcttctagaattccagcccgggcggatcccaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggctagcttagatcccagcttttgttccctttagtgagggttaatttcgagcttggcgtaatcgctagcggatctgacggttcactaaaccagctctgcttatatagacctcccaccgtacacgcctaacgcgctagccttgcatgcctgcaggtcggagtactgtcctccgagcggagtactgtcctccgagcggagtactgtcctccgagcggagtactgtcctccgagcggagactctagagggacgcgtatcgatggcgccagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgactctagagctgcaggcaagcttggtaccgagctcggatccactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacgcggccgctcccgttatttgcgctctgttcctgttaatcaacctctggattacaaaatttgtgaaagattgactggtattcttaactatgttgctccttttacgctatgtggatacgctgctttaatgcctttgtatcatgctattacttcccgtacggctttcattttctcctccttgtataaatcctggttgctgtctctttatgaggagttgtggcccgttgtcaggcaacgtggcgtggtgtgcactgtgtttgctgacgcaacccccactggttggggcattgccaccacctatcaactcctttccgggactttcgctttccccctccctattgccacggcggaactcattgccgcctgccttgcccgctgctggacaggggctcggctgttgggcactgacaattccgtggtgttgtcggggaagctgacgtcctttccatggctgctcgcctgtgttgccaactggattctgcgcgggacgtccttctgctacgtcccttcggccctcaatccagcggaccttccttcccgcggcctgctgccggttctgcggcctcttccgcgtcttcgccttcgccctcagacgagtcggatctccctttgggccgcctatcgattagggataacagggtaatggtacctttaagaccaatgacttacaaggcagctgtagatcttagccactttttaaaagaaaaggggggactggaagggctaattcactcccagggtctctctggttagaccagatctgagcctgggagctctctggctaactagggaacccactgcttaagcctcaataaagctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctgtgcgggagaacggagttctattatgactcaaatcagtctccccaagcattcggggatcagagtttttaaggataacttagtgtgtagggggccagtgagttggagatgaaagcgtagggagtcgaaggtgtccttttgcgccgagtcagttcctgggtgggggccacaagatcggatgagccagtttatcaatccgggggtgccagctgatccatggagtgcagggtctgcaaaatatctcaagcactgattgatcttaggttttacaatagtgatgttaccccaggaacaatttggggaaggtcagaatcttgtagcctgtagctgcatgactcctaaaccataatttcttttttgtttttttttttttatttttgagacagggtctcactctgtcacctaggctggagtgcagtggtgcaatcacagctcactgcagcctcaacgtcgtaagctcaagcgatcctcccacctcagcctgcctggtagctgagactacaagcgacgccccagttaatttttgtatttttggtagaggcagcgttttgccgtgtggccctggctggtctcgaactcctgggctcaagtgatccagcctcagcctcccaaagtgctgggacaaccggggccagtcactgcacctggccctaaaccataatttctaatcttttggctaatttgttagtcctacaaaggcagtctagtccccaggcaaaaagggggtttgtttcgggaaagggctgttactgtctttgtttcaaactataaactaagttcctcctaaacttagttcggcctacacccaggaatgaacaaggagagcttggaggttagaagcacgatggaattggttaggtcagatctctttcactgtctgagttataattttgcaatggtggttcaaagactgcccgcttctgacaccagtcgctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaactgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtactggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagcgcggggaggcagagattgcagtaagctgagatcgcagcactgcactccagcctgggcgacagagtaagactctgtctcaaaaataaaataaataaatcaatcagatattccaatcttttcctttatttatttatttattttctattttggaaacacagtccttccttattccagaattacacatatattctatttttctttatatgctccagttttttttagaccttcacctgaaatgtgtgtatacaaaatctaggccagtccagcagagcctaaaggtaaaaaataaaataataaaaaataaataaaatctagctcactccttcacatcaaaatggagatacagctgttagcattaaataccaaataacccatcttgtcctcaataattttaagcgcctctctccaccacatctaactcctgtcaaaggcatgtgccccttccgggcgctctgctgtgctgccaaccaactggcatgtggactctgcagggtccctaactgccaagccccacagtgtgccctgaggctgccccttccttctagcggctgcccccactcggctttgctttccctagtttcagttacttgcgttcagccaaggtctgaaactaggtgcgcacagagcggtaagactgcgagagaaagagaccagctttacagggggtttatcacagtgcaccctgacagtcgtcagcctcacagggggtttatcacattgcaccctgacagtcgtcagcctcacagggggtttatcacagtgcacccttacaatcattccatttgattcacaatttttttagtctctactgtgcctaacttgtaagttaaatttgatcagaggtgtgttcccagaggggaaaacagtatatacagggttcagtactatcgcatttcagggtggcccgagagctgcatccggagtatctagatggagttccgcgttacataacttacggtaaatggcccgcctggctgaccgcccaacgacccccgcccattgacgtcaataatgactgatgttcccatagtaacgccaatagggactttccattgacgctaatgggagtttgttttggcaccaaaatcaacgggactttccaaaatgtcgtaataaccccgccccgttgacgcaaatgggcggtaggcgtgtactctagaaggtctatataagcagagctcgtttagtgaaccgtttatactacttatctggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcacggtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcttgcggccggaattcacaaatggcagtattcatccacaattttaaaagaaaaggggggattggggggtacagtgcaggggaaagaatagtagacataatagcaacagacatacaaactaaagaattacaaaaacaaattactaaaaattcnaaaattttcgggtttattacagggacagcagagatccactttgggc

(19) United States
(12) Patent Application Publication
(10) Pub. No.: US 2016/0032252 A1
(43) Pub. Date: Feb. 4, 2016

(54) STRAIN OF DEFORMED WING VIRUS (DWV)

(71) Applicant: THE UNIVERSITY OF WARWICK, Coventry (GB)

(72) Inventors: David J. EVANS, Coventry, West Midlands (GB); Eugene RYABOV, Coventry, West Midlands (GB)

(73) Assignee: THE UNIVERSITY OF WARWICK, Coventry (GB)

(21) Appl. No.: 14/783,787

(22) PCT Filed: Apr. 15, 2014
(86) PCT No.: PCT/GB2014/051176

§ 371 Date: Oct. 9, 2015

(30) Foreign Application Priority Data
Apr. 15, 2013 (GB) 1306812.7
Aug. 6, 2013 (GB) 1314063.7

(51) Int. Cl. C12N 7/00; C07K 16/10; C12N 15/113; A01K 67/033; C12Q 1/70

(52) U.S. Cl. CPC C12N 7/00; A01K 67/0333; C12Q 1/701; C12N 15/1131; C07K 16/1009; C12N 2770/32021; C12N 2770/32022; A01K 2227/706; A01K 2217/05; C12N 2310/14; C12N 2320/30

(57) ABSTRACT

The invention is in the field of virology and relates to the deformed wing virus (DWV). A new strain of deformed wing virus (DWV) has been identified that is predominant in bees infested with Varroa mites. This particular strain of DWV can be used in diagnostics to identify at risk colonies. Also, inhibitors of the particular strain may be used in the treatment and/or prevention of DWV.

### FIELD OF THE INVENTION

The invention is in the field of virology and relates to the deformed wing virus (DWV) and the use of inhibitors of DWV to prevent and/or treat DWV infection in bees.

### BACKGROUND OF THE INVENTION

Honeybees are of great importance to the global economy, far surpassing their contribution in terms of honey production. In all, 52 of the world's 115 leading agricultural crops rely on honeybee pollination to some extent. These crops represent approximately 35% of the human diet.

Honeybee numbers have decreased in recent years. One factor contributing to this decrease is disease. In particular, honeybees are susceptible to a host of picorna-like viruses, including the closely related Acute Bee Paralysis Virus (ABPV), Kashmir Bee Virus (KBV), and Israeli Acute Paralysis Virus (IAPV). Viral infection can have a devastating effect on the bee population, resulting in high mortality rates and a decrease in the number of bees and colonies.

The mite, Varroa destructor, which feeds on honeybee haemolymph, originated in Asia and arrived in the UK in the 1980s. The Varroa mites are known to transmit bee viruses. Research has identified several viral pathogens of honeybees that are transmitted by Varroa mites, including the deformed wing virus (DWV). The DWV is so called because the virus causes the appearance of honeybees with characteristic wing deformities together with other developmental defects such as abdominal stunting within bee colonies. Infestation of honeybee colonies with Varroa results in a dramatic increase in DWV levels.

### SUMMARY OF THE INVENTION

The present inventors have identified a novel strain of deformed wing virus (DWV) that is surprisingly predominant in bees infested with Varroa mites. This novel strain of the invention comprises a recombinant genome containing the Varroa Desctructor Virus (VDV-1)-derived structural genes and the DWV-derived non-structural genes. The inventors have identified two predominant genomic sequences for the DWV strain. These two sequences differ only in the 5′ non-coding regions (NCRs). Thus, the regions of the two genomic sequences which code for all the viral proteins are identical. The two genomic sequences are set out in SEQ ID NOs: 1 and 2.

The novel strain of the invention is found at high concentrations within individual bees in Varroa-infested colonies, with other, highly divergent, strains of DWV present at much lower concentrations. Typically the newly identified strain is present at 1,000-10,000 times the concentration of the other DWV strains. This is in contrast to non-Varroa infested colonies, where, although a high diversity of DWV strains is observed, individual bees exhibit a much lower viral load. The particular strain of DWV identified by the inventors can be used in diagnostics to identify those colonies at risk of Varroa-transmitted deformed wing disease. Inhibitors of the particular strain may be used in the treatment and/or prevention of DWV-induced disease.

Also, transgenic bees can be generated which are resistant to the particular strain of DWV and hence resistant to Varroa mite-induced deformed wing disease.

Accordingly, the present invention provides a polynucleotide comprising (a) the sequence shown in SEQ ID NO: 1, 2, 52, 53, 54, 55, 56, 57, 58, 59, 60, 61 or 62, (b) a variant sequence having at least 98% homology to SEQ ID NO: 1, 2, 52, 53, 54, 55, 56, 57, 58, 59, 60, 61 or 62 based on nucleotide identity over its entire length or (c) a sequence that is complementary to the sequence in (a) or (b).

The invention also provides an oligonucleotide which specifically hybridises to a part of the polynucleotide of the invention.

The invention also provides an oligonucleotide which comprises 50 or fewer consecutive nucleotides from a polynucleotide of the invention.

The invention also provides a polynucleotide comprising at least two oligonucleotides of the invention.

The invention also provides an isolated strain of deformed wing virus (DWV) which comprises the varroa destructor virus 1 (VDV-1) capsid proteins (CP) and the DWV non-structural proteins (NS). This isolated strain of deformed wing virus (DWV) is referred to herein as the isolated DWV strain of the invention, or the isolated strain of the invention. This isolated strain may comprise a polynucleotide comprising (a) the sequence shown in SEQ ID NO: 1, 2, 52, 53, 54, 55, 56, 57, 58, 59, 60, 61 or 62, (b) a variant sequence having at least 98% homology to SEQ ID NO: 1, 2, 52, 53, 54, 55, 56, 57, 58, 59, 60, 61 or 62 based on nucleotide identity over its entire length or (c) a sequence that is complementary to the sequence in (a) or (b).

The invention also provides an antibody which specifically binds to an isolated strain of the invention.

The invention also provides a method of determining the presence or absence of an isolated strain of the invention in a sample, comprising (a) contacting the sample with an oligonucleotide of the invention or an antibody of the invention and (b) detecting specific hybridisation of the oligonucleotide or specific binding of the antibody and thereby determining the presence or absence of the isolated strain.

The invention also provides a method of determining the concentration of an isolated strain of the invention in a sample, comprising (a) contacting the sample with an oligonucleotide of the invention or an antibody of the invention and (b) detecting the amount of specific hybridisation of the oligonucleotide or the amount of specific binding of the antibody and thereby determining the concentration of the isolated strain.

The invention further provides a vector comprising a polynucleotide of the invention or an oligonucleotide of the invention, wherein said polynucleotide or oligonucleotide is operably linked to a promoter.

The invention further provides a composition comprising a polynucleotide of the invention, an oligonucleotide of the invention, an antibody of the invention and/or a vector of the invention and a delivery vehicle.

The invention also provides a method of treating or preventing in a bee or bee colony an infection with an isolated strain of the invention, comprising contacting the bee or bee colony with an inhibitor of the isolated strain.

The invention also a method of treating or preventing deformed wing disease in a Varroa mite-infested bee or bee colony, comprising contacting the bee or bee colony with an inhibitor of an isolated strain of the invention.

The invention also provides a method of diagnosing in a bee or bee colony infection with an isolated strain of the invention, comprising determining the presence or absence of the isolated strain, wherein the presence of the isolated strain is indicative of the presence of infection with the isolated strain and wherein the absence of the isolated strain is indicative of the absence of infection with the isolated strain.

The invention also provides a method of diagnosing deformed wing disease in a Varroa mite-infested bee or bee colony, comprising determining the presence or absence of an isolated strain of the invention, wherein the presence of the isolated strain is indicative of the presence of deformed wing and wherein the absence of the isolated strain is indicative of the absence of deformed wing disease.

The invention also provides a transgenic bee that is resistant to infection by an isolated strain of the invention. The invention also provides a transgenic bee that is resistant to Varroa-mite induced deformed wing disease, wherein at least one cell of the bee expresses an oligonucleotide of the invention.

The invention further provides a method of generating a transgenic queen bee that is resistant to infection by an isolated strain of the invention, comprising (a) incorporating an oligonucleotide of the invention or a polynucleotide of the invention into the genome of one or more bee germ cells; and (b) generating the queen bee from said one or more germ cells.

The invention also provides a method of generating a transgenic bee of the invention, comprising using a transgenic queen bee of the invention or a transgenic queen bee produced using a method of the invention to generate the bee.

The invention also provides a method of preventing in a bee or bee colony an infection with an isolated strain of the invention, comprising using a transgenic queen bee of the invention or a transgenic queen bee produced using a method of the invention to generate the bee or bee colony.

The invention further provides a method of preventing deformed wing disease in a Varroa mite-infested bee or bee colony, comprising using a transgenic queen bee of the invention or a transgenic queen bee produced using a method of the invention to generate the bee or bee colony.

The invention also provides a method of producing a bee or bee colony that is resistant to infection from an isolated strain of the invention, comprising using a transgenic queen bee of the invention or a transgenic queen bee produced using a method of the invention to generate the bee or bee colony.

The invention also provides one or more bee germ cells comprising an oligonucleotide of the invention, a polynucleotide of the invention or a vector of the invention.

The invention also provides a bee colony wherein at least 50% of the bees within the colony are resistant to infection by an isolated strain of the invention.

The invention further provides a bee colony wherein at least 50% of the bees within the colony are resistant to Varroa mite-induced deformed wing disease.

### DESCRIPTION OF THE FIGURES

FIG. 1: Schematic of exemplary envelope vector, packaging vector and transduction vector.

FIG. 2: Design of the frame transfer experiment and summary of treatments. Shown are treatment groups, the results of quantification of DWV-like viruses by real-time PCR, average Ct value±standard deviation (SD) and the range of Ct values.

FIG. 3: Quantification of the RNA sequences coding for the DWV and VDV-1 capsid protein (CP) and non-structural protein (NS). The numbers of RNA molecules per bee were quantified with the primers specific to DWV CP, DWV NS, VDV-1 CP, VDV-1 NS and primers recognising both DWV and VDV-1 NS (Universal NS) the VDV-1 CP and non-structural (NS) protein-coding sequences in the treatment group bee (n=8 for each group). Bars show mean value with standard error (SE). Letters above the bars represent statistically significant groupings according to pairwise t-tests comparisons, p-value <0.05; asterisk marks p-value <0.0001.

FIG. 4: Phylogenetic analysis of the central region of DWV strains, positions 4926 to 6255 of DWV GenBank Accession number AJ489744. The tip labels include GenBank accession numbers. The tip labels prefixes are as follows: C, NV, VL, VH denote corresponding honeybee pupae treatment group; Varroa-VH and Varroa-VL mark the sequences from Varroa mites associated with groups VH and VL respectively; “Infested-colony” denotes sequences derived from bees of the Varroa source colony; DWV, VDV-1, VDV-1-DWV-Rec followed by a place name indicate reference DWV, VDV-1 and VDV-1-DWV recombinant sequences present in GenBank. Sequences derived from the group VH bees are indicated with arrows. Sequences from the Varroa mites associated with the groups VH and VL are marked with filled or empty squares respectively. Alignments were performed using CLUSTAL (Thompson et al., 1997), and the neighbour-joining trees were produced and bootstrapped using the PHYLIP package programs (Felsenstein, 1989). Numbers at the nodes represent bootstrap obtained from 1000 replications shown for the major branches supported by more than 750 replications. The length of branches is proportional to the number of changes.

FIG. 5: Shows the results of high throughput sequencing of RNAi populations. The top graph shows the sense and anti-sense RNAs generated against DWV-derived sequences across all treatment groups. The middle graph shows the sense and anti-sense RNAs generated against VDV-1-derived sequences across all treatment groups. The bottom two graphs show the sense and anti-sense RNAs generated by the C and VH treatment colonies. The number of sense and anti-sense RNAs generated by each treatment group is summarised in Table 2.

FIG. 6: Shows profiles of the DWV-type and VDV-1-type siRNA for the RNAi libraries derived from the pooled samples of each of the C, NV, VH, and VL treatment groups.

FIG. 7: Shows a schematic of a transduction vector comprising the preferred promoter of the invention operably linked to a luciferase reporting gene. The graphs and photographs demonstrate that whole body transfection of bee larvae with the transduction vector resulted in luciferase expression by the transfected bee larva.

FIG. 8: Shows changes in the strain composition of DWV complexes in bee pupae following hemolymph injection. Levels of the DWV- and VDV-1 CP-coding RNA determined by qRT-PCR (Left panel) in the virus preparations used in the hemolymph injection, and (Right panel) in the progeny of the injected virus following 3 days of replication. (A) ΔCt values for the DWV-type and VDV-1-type CP were obtained by subtracting Ct values for the corresponding CP from Ct for the total DWV-like viruses quantified using “universal” primers to the NS gene. (B) Ct values for the DWV-type and VDV-1-type CP. Six bee pupae were used for each virus-injected group, three bee pupae were used for the buffer-injected and non-injected control groups. Bars show mean value with standard error (SE). Letters above the bars represent statistically significant groupings according to pairwise t-tests comparisons for VDV-1 CP (p-value <0.01). (B) High-throughput sequencing of the virus preparations from the bees of the treatment groups C, and the virus accumulated in the pupae injected with 20 ng of the virus preparation, 3 days post injection. The graphs show pileup numbers of the DWV and VDV-1 reads determined by a high-throughput sequencing of viral RNA aligning to the DWV and VDV-1 sequences (GeneBank Accession numbers GU109335 and AY251269 respectively) only reads unambiguously aligning to DWV or to VDV-1 sequences were used, up to 3 mismatches were allowed for the 18 nt seed region. The compositions of DWV complexes predicted and structures of the DWV-VDV-1 recombinants predicted by MosaicSolver are shown under the pileup graphs. The pileup graphs and the lines representing viral RNA regions for DWV and VDV-1 are shown. The CP-coding regions of the virus C preparation and the virus C-injected pupae, which shows decrease of the DWV coverage compared to the injected virus, are highlighted.

FIG. 9: Summary of the gene expression changes in the experiment. (A) Total number of differentially expressed (DE) genes in the contrasts. The numbers of up-regulated and down-regulated genes in each contrast are marked, respectively, as T and L. An up-regulated gene level is higher at the head of the arrow showing the contrast; commonality is shown in brackets. The numbers of overrepresented GO Biological Process terms associated with the DE genes are shown in red italic characters for each contrast. (B) A geometrical visualization of the three-stage experimental process: shown are, with numbers of differentially expressed genes, the “orthogonal” stages, contrasts C to NV (black), NV to VL (red), VL to VH (blue), and the commonalities in the composite stages shown in the colour of the “orthogonal” contrast. The DE gene numbers in the composite contrasts without commonalities to the “orthogonal” stages are shown in grey. Commonalities between orthogonal stages are shown in corresponding colour in brackets. (C) Result of principal component analysis applied to a set 60 DE genes (pooled from all contrasts) with low adjusted p-values. Shown is a plot of the first two principal component scores for Cy3 and Cy5 replicates for all honeybee samples.

FIG. 10: High-throughput sequencing of the honeybee small RNA libraries. The graphs show depth of coverage at the genomic loci of DWV (red) and VDV-1 (blue). A statistical summary of the reads is given to the right of each group. Only reads unambiguously aligning to DWV or VDV-1 were used (GenBank Accession numbers GU109335 and AY251269 respectively) with no mismatches being tolerated in the 18 nt. seed.

FIG. 11: Total and strain-specific virus genome quantification in honeybee pupae. Quantification of the viral RNA by qRT-PCR in the honeybee pupae from the frame transfer experiment. Numbers of the viral RNA molecules per pupa (n=8 for each experimental group) are shown. (A) Total DWV-like virus load quantified with the primers recognising the NS region of all DWV-like viruses (DWV, VDV-1, recombinants thereof and KV). (B) Quantification of the DWV CP, DWV NS, VDV-1 CP, and VDV-1 NS with the specific primers. Bars show mean value with standard error (SE). Letters above the bars represent statistically significant groupings according to pairwise t-test comparisons, p<0.05; asterisk marks p<0.0001.

FIG. 12: Phylogenetic analysis of the central region of DWV-like virus genome. PCR amplified cDNA was cloned and sequenced through the region corresponding to positions 4926 to 6255 of the DWV genome (GenBank Accession number AJ489744). The tip labels include GenBank accession numbers and are prefixed as follows: C, NV, VL, VH denote the corresponding honeybee pupae treatment group; Varroa-VH and Varroa-VL indicate sequences from Varroa mites associated with groups VH and VL respectively; “Infested-colony” denotes sequences derived from pupae from the Varroa source colony; DWV, VDV-1, VDV-1-DWV-Rec followed by a place name indicate reference DWV, VDV-1 and VDV-1-DWV recombinant sequences present in GenBank. Sequences derived from the group VH honeybee pupae are highlighted with arrows and sequences from Varroa mites associated with groups VH and VL are indicated with filled or empty squares respectively. Alignments were performed using CLUSTAL [77], and the neighbour-joining trees were produced and bootstrapped using the PHYLIP package [78]. Numbers at the nodes represent bootstrap values obtained from 1000 replications shown for the major branches supported by more than 750 replications. The length of branches is proportional to the number of changes. RF1 to RF4 indicate the distinct DWV/VDV-1 recombinant forms as defined by similarity to reference DWV and VDV-1 sequences (GenBank Accession numbers GU109335 and AY251269 respectively) in the CP and NS regions of the sequence. DWV^{V} indicates virulent form of DWV.

FIG. 13: Changes in the strain composition of DWV complexes in honeybee pupae following direct injection of virus. Levels of the DWV- and VDV-1 CP-coding RNA determined by qRT-PCR (left panel) in the virus preparations used for injection, and (right panel) in pupae following incubation for 3 days. (A) Left panel: ΔCt values for the DWV-type and VDV-1-type CP were obtained by subtracting Ct values for the corresponding CP from Ct for the total DWV-like viruses quantified using “Universal” primers to the NS gene. Right panel: Ct values for the DWV-type and VDV-1-type CP. Six pupae were used for each virus-injected group, three pupae were used for each of the buffer-injected and non-injected control groups. Bars show mean value with standard error. Letters above the bars represent statistically significant groupings according to pairwise t-test comparisons for VDV-1 CP (p-value <0.01). (B) High-throughput sequencing of the virus preparations from the honeybees of group C (left), and the virus accumulated in the pupae injected with 20 ng of the virus preparation (right), 3 days post injection. The graphs show depth of coverage at genomic loci in DWV (red) and VDV-1 (blue) determined by high-throughput sequencing of viral RNA aligning to the DWV and VDV-1 sequences (GeneBank Accession numbers GU109335 and AY251269 respectively). Only reads unambiguously aligning to DWV or VDV-1 sequences were used, with up to 3 mismatches tolerated in the 18 nt. seed region. The percentages of DWV, VDV-1 and the DWV-VDV-1 recombinants predicted by MosaicSolver [40] are shown below. The pileup graphs for DWV and VDV-1 are shown, respectively, in red and dark blue. The CP-coding region of the virus C preparation and the virus C-injected pupae, which shows a decrease of DWV coverage compared to the injected virus, is highlighted in yellow.

FIG. 14: DWV diversity and the level of DWV accumulation. Average Shannon's diversity Index (corrected for NGS sequencing error, as described in [44]) across the NS region, plotted against the proportion of DWV and VDV-1 reads. The error bar associated with each point is a 95% confidence interval for averages produced in this way. (B, C) Shannon's diversity index for all honeybees with low virus levels (groups “Control pupae”, “Buffer-injected pupae” and “Asymptomatic nurse honeybees”) and for the honeybees with high virus levels (groups “Virus-injected pupae” and “Symptomatic nurse honeybees”), (B) for the NS region and (C) for the CP region positions in the DWV reference genome, GenBank Accession number AJ489744, are 5008 to 9826 and 1751 to 4595 respectively. A 95% confidence interval for clonal input RNA library is shown as dashed line at 0.012. The sets of diversity values in (B) and (C) are significantly different, Least Significant Difference (LSD) test at 0.1%.

FIG. 15: Bimodal distribution of DWV accumulation in the experimental honeybee pupae. (A) Dotplot of Ct values by experimental group, determined by qRT-PCR, showing means and 95% confidence intervals for the means. The means for C, NV and VL are not significantly different. The difference between the mean of VH and the pooled C, NV and VL is significant with p-value <10⁻¹⁶. (B) A histogram shows bimodality of Ct values.

FIG. 16: Orthogonality of the differential gene expression pattern. A geometrical visualization of the three-stage experimental process. The first stage is “frame transfer” which includes exposure to Varroa-selected viruses through feeding at larval stage (contrast C to NV), the second stage is exposure to the Varroa mite feeding on the pupae haemolyph (contrast NV to VL) and the third stage is development of high viral load (contrast VL to VH). (A) Numbers of significantly differentially expressed genes in each of the three stages are shown alongside the directional vectors, together with numbers of differentially expressed genes in the composite stages (contrasts C to VL, NV to VH, C to VH). (B) The three stages involve distinct sets of differentially expressed genes, depicted in the graphic as orthogonality of the associated vectors; the very small number of genes common to pairs of contrasts are shown. (C) The large number of differentially expressed genes common to the pairs of non-orthogonal contrasts are shown.

FIG. 17: Principal component analysis (PCA) produced with 30 genes selected from the top genes from each contrast ranked by adjusted p-value. The genes were selected as follows: 7 top genes were selected from each of the 6 contrasts, and the 30 with the lowest adjusted p-values used in subsequent analysis. The scatterplot of the first two principal components for all honeybee samples (average for Cy3 and Cy5 replicates) is shown,

FIG. 18: Summary of numbers of differentially expressed immune-related genes. The number of up- and down-regulated genes in each contrast are marked, respectively, as ↑ and ↓. An up-regulated gene level is higher at the head arrow showing the contrast; commonality is shown in brackets.

FIG. 19: Correlation between the virus levels in honeybee pupae and the corresponding mites. Two-dimensional plots showing the results of the qRT-PCR quantification of viral RNA in the honeybee pupae (log₁₀ transformed copy number of the viral RNA per honeybee) and the corresponding Varroa mites (log₁₀ transformed viral RNA copy number normalised to Varroa (3-actin copy number) from experiment groups VL and VH. Panels shows results of (A) total DWV-like virus quantified with the primers recognising the NS region of DWV, VDV-1 and KV, then specific quantification of (B) VDV-1 CP, (C) VDV-1 NS, (D) DWV CP and (E) DWV NS regions.

FIG. 20: Quantification of negative strands of DWV RNA in the Varroa mites of the groups VH and VL. The graph shows average copy number per mite of DWV- and VDV-1-like CP-coding sequence as determined by negative-strand specific qRT-PCR using primers listed in Table 1. The dotted line indicates the detection threshold as determined by a water-only control plus two standard deviations.

FIG. 21: Genetic diversity of DWV in the honeybee groups. Average Shannon's diversity index values. (A) the CP region positions 1751 to 4595, (B) the NS region, positions 5008 to 9826, and the central region, positions 5250 to 6250. Positions are given for the reference DWV genome, GenBank Accession number AJ489744. Average Shannon's index was calculated for five random 3285-read samples from the viral reads for each NGS library of individual bees. Bars indicate SD. Letters above the bars represent statistically significant groupings according to Fisher's Least Significant Difference (LSD) test as 5% and 0.1% levels, marked with * and *** respectively. The dashed lines indicate the average Shannon's diversity index values for the NGS sequencing error, ±standard deviation (SD). In panel (C) the dotted line at 0.0417 marks the Shannon's diversity index for Group NV of the frame transfer experiment.

### DESCRIPTION OF SEQUENCES

SEQ ID NOs: 1 and 2 are the genomic sequences of the DWV strain of the invention. As discussed herein, the regions encoding the structural proteins and non-structural proteins are identical between SEQ ID NOs: 1 and 2. Only the sequence of the 5′ non-coding region (NCR) differs between SEQ ID NOs: 1 and 2.

SEQ ID NO: 3 is the coding sequence for the Varroa-destructor virus-1 (VDV-1) capsid proteins (CP).

SEQ ID NO: 4 is the coding sequence for the DWV non-structural proteins (NS).

SEQ ID NOs: 5 and 6 are the sequences comprised in the preferred polynucleotides of the invention. Preferred oligonucleotides of the invention may be derived from these sequences.

SEQ ID NO: 7 is the sequence of a preferred transducing vector, pTYF-mCMW/SYN-EGFP.

SEQ ID NO: 8 is the sequence of the newly-identified honeybee heat shock protein 70 (hsp70) promoter.

SEQ ID NO: 9 is the sequence of a preferred honeybee transcription terminator.

SEQ ID NO: 10 is the sequence of a transducing vector derived from pTYF-mCMW/SYN-EGFP which comprises a luciferase reporter gene operably linked to the honeybee hsp70 promoter of SEQ ID NO: 7.

SEQ ID NOs: 11 to 51 are sequences of exemplary oligonucleotides of the invention (Table 1).

SEQ ID NO: 52 is the sequence of Clone-202-complete-KJ437447.

SEQ ID NO: 53 is the sequence of Clone-complete-204

SEQ ID NO: 54 is the sequence of Clone-partial-21

SEQ ID NO: 55 is the sequence of Clone-23-partial

SEQ ID NO: 56 is the sequence of NGS-Library-E7

SEQ ID NO: 57 is the sequence of NGS-Library-E8

SEQ ID NO: 58 is the sequence of NGS-Library-E10

SEQ ID NO: 59 is the sequence of NGS-Library-E11

SEQ ID NO: 60 is the sequence of NGS-Library-INJ4

SEQ ID NO: 61 is the sequence of NGS-Library-INJ5

SEQ ID NO: 62 is the sequence of NGS-Library-INJ6

SEQ ID NOs: 52 to 55 are PCR amplified cDNAs that have been sequenced by standard Sanger sequencing.

SEQ ID NOs: 56 to 62 are consensus sequences determined from next generation sequencing, either of naturally infected bees (SEQ ID NOs: 56 to 59) or honeybee pupae injected with virus in the laboratory (SEQ ID NOs: 60 to 62).

SEQ ID NOs: 63 to 72 are sequences of exemplary oligonucleotides of the invention (Table 5).

SEQ ID NO: 73 is the sequence of a preferred transducing vector, pTYF-mCMW/SYN-EGFP.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more such polynucleotides, reference to “an oligonucleotide” includes two or more such oligonucleotides, reference to “a virus” includes two or more such viruses, and the like.

All publications, patents and patent applications cited herein, whether supra or infra, are hereby incorporated by reference in their entirety.

### Deformed Wing Virus

Deformed wing virus (DWV), Varroa destructor virus-1 (VDV-1) (Genbank Accession No. NC_{—}006494, Version No. NC_{—}006494.1 GI:56121875, Ongus et al 2004 J. Gen. Virol. 85: 3747-3755), their recombinants, and Kakugo virus (KV) (Genbank Accession No. NC_{—}005876, Version No. NC_{—}005876.1 GI:47177088, Fujiyuki et al 2004 J. Virol. 78(3): 1093-1100) are closely related picorna-like RNA viruses infecting the European honeybee (Apis mellifera). Some of these viruses also infect the ectoparasitic mite Varroa destructor. The overall nucleotide homology between the viruses related to DWV is no less than 84%. Therefore, for the purposes of the present invention, these viruses are considered as strains of the same virus, DWV.

Picorna-like viruses, including DWV, possess genomes that are essentially ‘modular’. These modular genomes consist of 4 modules. From the 5′ end (i.e. the direction of translation and the direction that they are always represented in print) the four modules are:
1) a 5′ non-coding region (NCR) module which is involved in genome replication and translation of the virus polyprotein;
2) a structural protein module which encodes the virus capsid or coat proteins;
3) a non-structural protein module which encodes the proteins involved in a) replication of the virus genome and b) interaction with the cellular environment to allow virus replication to occur; and
4) a 3′ NCR module which is almost exclusively involved in replication of the virus genome.

The individual modules can be recombined to create recombinant viral genomes.

Recombinant viruses must have all four modules in the correct order, and the modules must be compatible with each other. Module compatibility is achieved through selection, as only functional viral genomes will allow viral growth and the generation of progeny viruses.

DWV is a single-stranded, positive sense RNA virus. Prior to the present invention, DWV has been relatively poorly characterised. The DWV genome is approximately 10150 nucleotides in length and has a modular architecture as discussed above. In particular, the four DWV modules have been previously defined as follows (Genbank Accession No. NC_{—}004830; Version No. NC_{—}004830.2 GI:71480055, Lanzi et al. 2006 J. Virol. 80(10): 4998-5009):
nucleotides 1-1117: the 5′ NCR module;
nucleotides 1118-4594: the structural protein module;
nucleotides 4595-9799: the non-structural protein module; and
nucleotides 9798-end: the 3′ NCR module.

In the DWV genome there is an additional protein, the L protein (a non-structural protein) which is coded before the first of the structural proteins. Although other strains of DWV are known in the art, the present strain is newly identified by the inventors.

DWV is present in the majority of honeybee colonies, but in the absence of its ectoparasite, the mite V. destructor which feeds on the bee haemolymph, DWV generally causes asymptomatic infection and accumulates to low concentrations in infected bees. Conversely, in Varroa-infested colonies bees show impaired development and increased mortality associated with very high DWV concentrations.

The present inventors have investigated the effect of Varroa infestation on the DWV strain composition and DWV concentration in DWV infected colonies. Pupae infected orally but remaining mite-free showed significant changes in DWV strain composition, but only a slight increase in overall DWV concentrations. Varroa-infestation resulted in further changes in DWV strain compositions with concentrations of DWV showing bimodal distribution, being either similar to the orally infested or 1000-times higher. Surprisingly, the present inventors found that in bees with the very highest concentrations of DWV-like viruses, which comprise about 80% of the Varroa mite-infested bee pupae, there was a single recombinant strain of DWV/VDV. The predominant (up to 99.9% of the virus genomes present) strain in the Varroa-infested bees had recombinant genomes containing the VDV-1-derived structural genes and the DWV-derived non-structural genes.

Accordingly, the present invention provides an isolated strain of DWV comprising a recombinant genome containing the VDV-1-derived structural genes and the DWV-derived non-structural genes. In one embodiment, in the isolated strain of the invention the precise recombination junction between the VDV-1 derived sequence and the DWV-derived sequence matches exactly to the junction of the VDV-1-derived structural genes and the DWV-derived non-structural genes. In another embodiment, the precise recombination junction between the VDV-1 derived sequence and the DWV-derived sequence does not matches exactly to the junction of the VDV-1-derived structural genes and the DWV-derived non-structural genes. For example, the recombination junction may lie within 100 nucleotides, within 50 nucleotides, within 20 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides or within 1 nucleotide of the junction of the structural genes and the non-structural genes.

The present inventors have sequenced the genome of the isolated DWV strain and have identified two predominant sequences. The sequences of the isolated strain are set out in SEQ ID NOs: 1 and 2. SEQ ID NO: 1 comprises (1) the DWV 5′ NCR module; (2) a structural protein module encoding the varroa destructor virus 1 (VDV-1) capsid proteins (CP); (3) a non-structural protein module encoding the DWV non-structural proteins (NS); and (4) a 3′ NCR. SEQ ID NO: 2 comprises (1) the VDV-1 5′ NCR module; (2) a structural protein module encoding the VDV-1 CP; (3) a non-structural protein module encoding the DWV NS; and (4) a 3′ NCR.

The VDV-1 CP are encoded by SEQ ID NO: 3, which is present in both SEQ ID NO: 1 and 2. The DWV NS are encoded by SEQ ID NO: 4, which is present in both SEQ ID NO:1 and 2. SEQ ID NO: 3 is found at nucleotides 1118 to 4594 of SEQ ID NO: 1 and at nucleotides 1145 to 4621 of SEQ ID NO: 2. SEQ ID NO: 4 is found at nucleotides 4595 to 9799 of SEQ ID NO: 1 and at nucleotides 4622 to 9826 of SEQ ID NO: 2. Corresponding regions in SEQ ID NO: 52, 53, 54, 55, 56, 57, 58, 59, 60, 61 or 62 can be determined using sequence alignments. Methods of aligning sequences are discussed in more detail below.

The sequences of SEQ ID NOs: 1 and 2 differ in their 5′ NCR. The regions of the two genomic sequences which code for all the viral proteins (approximately 90% of the genome) are identical. Thus, the isolated strain of the invention can be thought of as possessing a single defined protein coding region with one of two alternative 5′ NCRs. It is the particular combination of protein coding regions of the isolate strain, i.e. modules 2 and 3 identified above, which give the isolated strain of the invention its unique characteristics. The 5′ NCR is not determinative. Therefore, the strain of the invention may comprise the NCR of either DWV or VDV-1, such that translation of the strain of the invention is mediated by the 5′ NCR of either DWV or VDV-1.

In one embodiment the isolated strain of the invention comprises a polynucleotide comprising a non-structural protein module encoding the DWV NS and/or a structural protein module encoding the VDV-1 CP. In a preferred embodiment the isolated strain comprises a polynucleotide comprising both a non-structural protein module encoding the DWV NS and a structural protein module encoding the VDV-1 CP.

The VDV-1 CP is the capsid protein region of the DWV strain of the invention. The capsid proteins form the outer proteins of the virus particle and are therefore considered to be structural proteins (Lanzi et al. 2006 J. Virol. 80(10): 4998-5009). The VDV-1 CP comprises 4 capsid proteins, VP1, VP2, VP3 and VP4 which are expressed as part of a polyprotein, preceded by protein L.

The DWV NS is the non-structural protein region of the DWV strain of the invention. This region comprises: (1) a helicase that is predicted to have RNA structure unwinding activity; (2) a viral protein genome linked (VPg) protein which covalently attaches to the viral genome during replication; (3) a second protease similar to the 3C protease of other picornaviruses; and (4) an RNA-dependent RNA polymerase (RdRp).

In one embodiment, the isolated strain of the invention comprises a polynucleotide sequence of SEQ ID NO: 3, which encodes the VDV-1 CP and/or a polynucleotide sequence of SEQ ID NO: 4, which encodes the DWV NS.

In a preferred embodiment, the isolated strain of the invention comprises a polynucleotide sequence of the invention as discussed below. The polynucleotide sequence of the isolated strain is preferably SEQ ID NO: 1, 2, 52, 53, 54, 55, 56, 57, 58, 59, 60, 61 or 62. The polynucleotide sequence of the isolated strain may be any of the variant sequences discussed below.

The strain of the invention is “isolated” in the sense that it is isolated (or separated) from its natural state and the molecules with which it is found in nature. The virus may be mixed with other molecules, carriers or diluents, such as those disclosed below, which will not interfere with its intended use. Typically, the isolated virus is not present in a bee or a Varroa mite.

An isolated strain of the virus may be produced by expressing a polynucleotide of the invention are discussed below.

### Polynucleotides and Oligonucleotides

The invention provides a polynucleotide comprising (a) the sequence of the genome of the isolated strain of the invention, namely SEQ ID NO: 1, 2, 52, 53, 54, 55, 56, 57, 58, 59, 60, 61 or 62, (b) a variant sequence having at least 98% homology to SEQ ID NO: 1, 2, 52, 53, 54, 55, 56, 57, 58, 59, 60, 61 or 62 based on nucleotide identity over its entire length or (c) a sequence which is complementary to SEQ ID NO: 1, 2, 52, 53, 54, 55, 56, 57, 58, 59, 60, 61 or 62 or a variant sequence as defined in (b).

A polynucleotide, such as a nucleic acid, is a polymer comprising two or more nucleotides. The nucleotides can be naturally occurring or artificial.

A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light.

One or more nucleotides in the polynucleotide may be modified, for instance with a label or a tag. The label may be any suitable label which allows the polynucleotide to be detected. Suitable labels include, but are not limited to fluorescent molecules, radioisotopes, e.g. ¹²⁵I, ³⁵S, enzymes, antibodies, antigens, other polynucleotides and ligands such as biotin.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide may be double stranded. The polynucleotide is preferably single stranded. The polynucleotide may be one strand from a double stranded polynucleotide.

The polynucleotide can be nucleic acids, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may comprise any of the nucleotides discussed above, including the modified nucleotides. The polynucleotide of the invention is preferably RNA.

SEQ ID NOs: 1 and 2 encode an isolated strain of DWV as discussed above. The VDV-1 CP are encoded by SEQ ID NOs: 3 and 4 within SEQ ID NO: 1 and 2 as discussed above. SEQ ID NOs: 52 to 55 are PCR amplified cDNAs that have been sequenced by standard Sanger sequencing. SEQ ID NOs: 56 to 62 are consensus sequences determined from next generation sequencing, either of naturally infected bees (SEQ ID NOS: 56 to 59) or honeybee pupae injected with virus in the laboratory (SEQ ID NOS: 60 to 62).

The polynucleotide of the invention may comprise a variant sequence based on SEQ ID NO: 1, 2, 52, 53, 54, 55, 56, 57, 58, 59, 60, 61 or 62. A variant sequence is a polynucleotide that has a nucleotide sequence which varies from that of SEQ ID NO: 1, 2, 52, 53, 54, 55, 56, 57, 58, 59, 60, 61 or 62 and which typically retains its ability to encode an isolated strain of the invention. The variant sequence preferably retains the ability to encode an isolated strain which comprises the varroa destructor virus 1 (VDV-1) capsid proteins (CP) and the DWV non-structural proteins (NS). The ability of a variant sequence to encode such a virus can be assayed using any method known in the art. For instance, the polynucleotide may be expressed as discussed below.

The variant sequence may comprise any of the nucleotides discussed above, including the modified nucleotides. The variant sequence is typically the same length as SEQ ID NO: 1, 2, 52, 53, 54, 55, 56, 57, 58, 59, 60, 61 or 62, but may be longer or shorter.

Over the entire length of the polynucleotide sequence of SEQ ID NO: 1, 2, 52, 53, 54, 55, 56, 57, 58, 59, 60, 61 or 62, a variant sequence is at least 98% homologous to that sequence based on nucleotide identity. The variant sequence may be at least 98% homologous to the region of SEQ ID NO: 1, 2, 52, 53, 54, 55, 56, 57, 58, 59, 60, 61 or 62 that comprises the structural protein module and/or the non-structural protein module of the DWV strain of the invention. Typically, the variant sequence may be at least 98% homologous to the region of SEQ ID NO: 1, 2, 52, 53, 54, 55, 56, 57, 58, 59, 60, 61 or 62 that comprises the structural protein module and the non-structural protein module of the DWV strain of the invention.

More preferably, the variant sequence may be at least 98.1%, at least 98.2%, at least 98.3%, at least 98.4%, at least 98.5%, at least 98.6%, at least 98.7%, at least 98.8%, at least 98.9%, at least 98.95%, at least 98.96% at least 98.97% at least 98.98% at least 98.99%, at least 99%, at least 99.1%, at least 99.2%, at least 99.3%, at least 99.4%, at least 99.5%, at least 99.6%, at least 99.7%, at least 99.8%, at least 99.9%, at least 99.95%, at least 99.96%, at least 99.97%, at least 99.98% or at least 99.99% homologous based on nucleotide identity to SEQ ID NO: 1, 2, 52, 53, 54, 55, 56, 57, 58, 59, 60, 61 or 62 over the region of SEQ ID NO: 1, 2, 52, 53, 54, 55, 56, 57, 58, 59, 60, 61 or 62 that comprises the structural protein module and the non-structural protein module of the DWV strain of the invention. The variant sequence may be at least 98.1%, at least 98.2%, at least 98.3%, at least 98.4%, at least 98.5%, at least 98.6%, at least 98.7%, at least 98.8%, at least 98.9%, at least 98.95%, at least 98.96% at least 98.97% at least 98.98% at least 98.99%, at least 99%, at least 99.1%, at least 99.2%, at least 99.3%, at least 99.4%, at least 99.5%, at least 99.6%, at least 99.7%, at least 99.8%, at least 99.9%, at least 99.95%, at least 99.96%, at least 99.97%, at least 99.98% or at least 99.99% homologous based on nucleotide identity to SEQ ID NO: 1, 2, 52, 53, 54, 55, 56, 57, 58, 59, 60, 61 or 62 over its entire sequence (i.e. over its entire length). There may be at least 99%, for example at least 99.5%, at least 99.8%, at least 99.9% or 100%, nucleotide identity over a stretch of 1000 or more, for example 2000, 3000, 4000, 5000, 6000, 7000, 8000 or 9000 or more, contiguous nucleotides of SEQ ID NO: 1, 2, 52, 53, 54, 55, 56, 57, 58, 59, 60, 61 or 62 (“hard homology”).

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p 387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

One of skill in the art is capable of generating sequences which are complementary to SEQ ID NO: 1, 2, 52, 53, 54, 55, 56, 57, 58, 59, 60, 61 or 62 or a variant sequence using canonical base pairing. The complementary sequence may comprise any of the nucleotides discussed above, including the modified nucleotides.

The invention also provides an oligonucleotide which specifically hybridises to a part of a polynucleotide of the invention, i.e. an oligonucleotide which specifically hybridises to part of (a) SEQ ID NO: 1, 2, 52, 53, 54, 55, 56, 57, 58, 59, 60, 61 or 62, (b) a variant sequence as defined above or (c) a sequence which is complementary to (a) or b). Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide of the invention is preferably 21 or 22 nucleotides in length. The oligonucleotide may comprise any of the nucleotides discussed above, including the modified nucleotides. The oligonucleotide may be double stranded. The oligonucleotide is preferably single stranded. The oligonucleotide is preferably RNA.

An oligonucleotide of the invention specifically hybridises to a part of a polynucleotide of the invention, hereafter called the target sequence. The length of the target sequence typically corresponds to the length of the oligonucleotide. For instance, a 21 or 22 nucleotide oligonucleotide typically specifically hybridises to a 21 or 22 nucleotide target sequence. The target sequence may therefore be any of the lengths discussed above with reference to the length of the oligonucleotide. The target sequence is typically consecutive nucleotides within the polynucleotide of the invention.

The target sequence is preferably present within the region of SEQ ID NO: 1, 2, 52, 53, 54, 55, 56, 57, 58, 59, 60, 61 or 62 that codes for structural proteins or non-structural proteins. In particular, the target sequence is preferably present in the region of SEQ ID NO: 1 or 2 that encodes the VDV-1 CP or the DWV NS, i.e. within SEQ ID NO: 3 or SEQ ID NO: 4. The target sequence is preferably in a corresponding region in SEQ ID NO: 52, 53, 54, 55, 56, 57, 58, 59, 60, 61 or 62. These can be determined using standard sequence alignment.

An oligonucleotide “specifically hybridises” to a target sequence when it hybridises with preferential or high affinity to the target sequence but does not substantially hybridise, does not hybridise or hybridises with only low affinity to other sequences, for example other sequences in SEQ ID NO: 1 or sequences from other strains of DWV (i.e. strains outside the scope of the present invention). For instance, an oligonucleotide “specifically hybridises” to the VDV-1 CP coding sequence when it hybridises with preferential or high affinity to that sequence (SEQ ID NO: 3) but does not substantially hybridise, does not hybridise or hybridises with only low affinity to other sequences in SEQ ID NO: 1, such as the DWV NS coding sequence in SEQ ID NO: 1 (SEQ ID NO: 4).

An oligonucleotide “specifically hybridises” if it hybridises to the target sequence with a melting temperature (Tm) that is at least 2° C., such as at least 3° C., at least 4° C., at least 5° C., at least 6° C., at least 7° C., at least 8° C., at least 9° C. or at least 10° C., greater than its Tm for other sequences in the HCV genome. More preferably, the oligonucleotide hybridises to the target sequence with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for other nucleic acids. Preferably, the portion hybridises to the target sequence with a Tm that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tm for a sequence which differs from the target sequence by one or more nucleotides, such as by 1, 2, 3, 4 or 5 or more nucleotides. The portion typically hybridises to the target sequence with a Tm of at least 90° C., such as at least 92° C. or at least 95° C. Tm can be measured experimentally using known techniques, including the use of DNA microarrays, or can be calculated using publicly available Tm calculators, such as those available over the internet.

More preferably, the oligonucleotide does not hybridise to sequences from other DWV strains or sequences from picorna-like viruses to which bees are susceptible, even under high stringency conditions. Most preferably, the oligonucleotide does not hybridise to any other nucleic acid even under high stringency conditions or sequences from picorna-like viruses to which bees are susceptible.

Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1× (0.1650 M Na+) to 2× (0.33 M Na+) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na+) to 1× (0.1650 M Na+) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na+) SSC at 60° C.

The oligonucleotide of the invention may comprise a sequence which is substantially complementary to the target sequence. Typically, the oligonucleotides are 100% complementary. However, lower levels of complementarity may also be acceptable, such as 95%, 90%, 85% and even 80%. Complementarity below 100% is acceptable as long as the oligonucleotides specifically hybridise to the target sequence. An oligonucleotide may therefore have 1, 2, 3, 4, 5, 6, 7, 8, 9, 10 or more mismatches across a region of 5, 10, 15, 20, 21, 22, 30, 40 or 50 nucleotides. Preferably 100% complementarity is present at positions in the target sequence that are unique to the isolated strain of the invention.

The oligonucleotide of the invention preferably comprises a sequence derived from SEQ ID NO: 5 or 6.

SEQ ID NO: 5
tagtagtccaagacgttataggagagttaagtcaggctatacccgatctt
caacaaccggaagttcaagcgaatgttttttctctggtgtcacagttagt
g
SEQ ID NO: 6
ttcaattatcaacgacacagttaatgaggaaaaagggaataaaacctcac
actatattcacggattgtttgaaagatacttgtttgcctgttgaaaaatg
tagaatacctggtaagactagaatatttagcataagtccggtgcagttta
ccataccgtttcgacagtattatttagactttatggcatcctatcgagct
gcacgacttaatgctgagcatggtattggtattgatgttaacagcttaga
gtggacaaatttggcaacaaggttgtctaagtatggcactcacatcgtga
caggagactataagaattttggtcctgggttagattccgatgttgcagct

The invention also provides an oligonucleotide which comprises 50 or fewer consecutive nucleotides from a polynucleotide of the invention, i.e. an oligonucleotide which comprises 50 or fewer consecutive nucleotides from (a) SEQ ID NO: 1, 2, 52, 53, 54, 55, 56, 57, 58, 59, 60, 61 or 62, (b) a variant sequence as defined above or (c) a sequence which is complementary to (a) or (b). The oligonucleotide may be any of the lengths discussed above. It is preferably 21 or 22 nucleotides in length. The oligonucleotide may comprise any of the nucleotides discussed above, including the modified nucleotides. The oligonucleotide preferably comprises or consists of any one of SEQ ID NOs: 11 to 51 to 63 to 72. The oligonucleotide may be double stranded. The oligonucleotide is preferably single stranded. The oligonucleotide is preferably RNA.

The oligonucleotide may derived from any region of a polynucleotide of the invention. In particular, the oligonucleotide of the invention may be any of the target sequences discussed above. The oligonucleotide is preferably derived from within the region of SEQ ID NO: 1, 2, 52, 53, 54, 55, 56, 57, 58, 59, 60, 61 or 62 that codes for structural proteins or non-structural proteins. The oligonucleotide of the invention is more preferably derived from the region of SEQ ID NO: 1 that encodes that encodes the VDV-1 CP or the DWV NS, i.e. from within SEQ ID NO: 3 or SEQ ID NO: 4. The oligonucleotide of the invention is more preferably derived from the region of SEQ ID NO: 2, 52, 53, 54, 55, 56, 57, 58, 59, 60, 61 or 62 that encodes that encodes the VDV-1 CP or the DWV NS.

The invention also provides a polynucleotide which comprise two or more oligonucleotides of the invention. The polynucleotide may be used to generate multiple oligonucleotides of the invention within a bee cell. Typically the polynucleotide is processed by the bee cell to produce two or more oligonucleotides of the invention. The polynucleotide may be processed by the bee cell to produce two or more oligonucleotides of the invention without maintenance of the polynucleotide within the bee cell. These resultant oligonucleotides may then generate a small interfering RNA (siRNA or RNAi) effect as discussed below.

Alternatively, the polynucleotide of the invention may be maintained within a bee cell. For example, the polynucleotide of the invention may be reversed-transcribed within the bee cell and the resulting DNA molecule either imported into the nucleus and integrated into the bee genome or maintained as extra-chromosomal DNA. The DNA can then be transcribed by the bee cell to produce two or more oligonucleotides of the invention, which may be useful in generating a RNAi effect as discussed below.

In a preferred embodiment, the polynucleotide of the invention comprises at least 2, at least 3, at least 4, at least 5, at least 10 or more oligonucleotides of the invention. The polynucleotides of the invention may be used to reduce the likelihood of resistance occurring within the isolated DWV strain as a result of the selection of single point mutations. The use of a polynucleotide comprising more than one oligonucleotide of the invention may give rise to an increased RNAi immune response compared with the use of a single oligonucleotide. This is discussed in more detail below.

In a preferred embodiment, the polynucleotide of the invention may comprise the sequence of SEQ ID NO: 5 and/or SEQ ID NO: 6.

As will be clear from the discussion below, the polynucleotides and oligonucleotides of the invention may be useful in generating an RNAi immune response against the isolated strain of the invention. The polynucleotide or oligonucleotide of the invention is preferably capable of generating an RNAi immune response to the isolated strain of the invention in a bee cell. This RNAi immune response typically leads to gene silencing within the isolated strain. Gene silencing in bees using oligonucleotides has been demonstrated in the art in relation to other bee viruses (Maori et al., Insect Molecular Biology, 2009; 18(1): 55-60; and Hunter et al., PLoS Pathogens, 2010; 6(12): e1001160). Typically an RNAi immune response involves the generation of dsRNA molecules or hairpin RNA molecules. The mechanism of action of such RNA molecules is well characterised in the art. The polynucleotide or oligonucleotide of the invention may be derived from any region to be silenced, including but not limited to, the coding sequence of the gene to be silenced, the 5′ untranslated region, the 3′ untranslated region, the promoter of the gene to be silenced, or any combination thereof.

More preferably the generated RNAi immune response is specific to the isolated strain of the invention. An RNAi immune response may be obtained when the polynucleotide or oligonucleotide is applied exogenously to a bee or bee cell. Exogenous application of the polynucleotide or oligonucleotide may or may not lead to maintenance of the polynucleotide or oligonucleotide in the bee or bee cell. Specifically, exogenous application of the polynucleotide or oligonucleotide may or may not lead to integration of the polynucleotide or oligonucleotide into the bee genome. A polynucleotide of the invention may be delivered to a bee or bee cell. Preferably the polynucleotide will be processed to generate two or more dsRNA molecules comprising oligonucleotides of the invention. These dsRNA molecules may then generate an RNAi immune response.

Alternatively, upon delivery the polynucleotide or oligonucleotide of the invention may be maintained in a bee or bee cell. The polynucleotide or oligonucleotide of the invention may be maintained as a stable genetic element outside the bee genome, i.e. the polynucleotide or oligonucleotide may be maintained within the bee or bee cell without integration into the bee genome. The polynucleotide or oligonucleotide of the invention may be maintained by being integrated into the bee genome. Preferably, the polynucleotide or oligonucleotide of the invention may be capable of acting as described in Goic et al. (Nature Immunology 2013: 14(4): 396-403). Specifically, in a preferred embodiment, the polynucleotide or oligonucleotide is capable of being reverse-transcribed in a bee cell, resulting in DNA forms embedded in retrotransposon sequences. The virus-retrotransposon DNA chimeras may then produce transcripts that are processed by the RNAi machinery within the bee cell, which in turn inhibits replication of the isolated strain. This is discussed in more detail below.

Thus, in generating an RNAi immune response, the polynucleotide or oligonucleotide of the invention is preferably capable of reducing the level of the isolated strain of the invention in a bee cell, bee or bee colony.

The invention also provides a method of generating a polynucleotide or oligonucleotide of the invention derived from SEQ ID NO: 1, 2, 52, 53, 54, 55, 56, 57, 58, 59, 60, 61 or 62. Typically the method involves the design and/or production of primers specific to the target sequence in SEQ ID NO: 1, 2, 52, 53, 54, 55, 56, 57, 58, 59, 60, 61 or 62 and using the specific primers to amplify the target sequence. Specific primers can be designed and generated using standard techniques known in the art. Similarly, methods for amplifying target sequences using such primers are also known in the art. A polynucleotide or oligonucleotide of the invention derived from SEQ ID NO: 1, 2, 52, 53, 54, 55, 56, 57, 58, 59, 60, 61 or 62 may also be generated synthetically using known techniques.

### Antibodies

The invention provides an antibody which specifically binds to the isolated strain of the invention.

Screening methods are well known to those of skill in the art which may be used to generate and identify antibodies that are capable of specifically binding to the isolated strain of the invention (e.g. Making monoclonals: A practical beginners' guide to the production and characterization of monoclonal antibodies against bacteria and viruses, Newell, Public Health Laboratory Service (1988), ISBN-13: 978-0901144232). Antibodies of the invention can be tested for specific binding to an isolated strain of the invention by, for example, standard ELISA or Western blotting. An ELISA assay can also be used to screen for hybridomas that show positive reactivity with the strain. The binding specificity of an antibody may also be determined by monitoring binding of the antibody to the virus, for example by flow cytometry.

Antibodies of the invention will specifically bind to antigens and epitopes within the isolated strain of the invention. The antigens and epitopes may be identified and used to prepare additional antibodies of the invention.

An antibody “specifically binds” or “specifically recognises” an isolated strain of the invention when it binds with preferential or high affinity to the strain for which it is specific but does not substantially bind, or binds with low affinity, to other viruses or proteins. The specificity of an antibody of the invention for the isolated strain may be further studied by determining whether or not the antibody binds to other related strains or whether it discriminates between them.

An antibody of the invention binds with preferential or high affinity if it binds with a Kd of 1×10⁻⁷ M or less, more preferably 5×10⁻⁸ M or less, more preferably 1×10⁻⁸ M or less or more preferably 5×10⁻⁹ M or less. An antibody binds with low affinity if it binds with a Kd of 1×10⁻⁶ M or more, more preferably 1×10⁻⁵ M or more, more preferably 1×10⁻⁴ M or more, more preferably 1×10⁻³ M or more, even more preferably 1×10⁻² M or more. A variety of protocols for competitive binding or immunoradiometric assays to determine the specific binding capability of antibodies are well known in the art (see for example Maddox et al, J. Exp. Med. 158, 1211-1226, 1993).

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

An antibody of the invention may be a monoclonal antibody or a polyclonal antibody, and will preferably be a monoclonal antibody. An antibody of the invention may be a chimeric antibody, a CDR-grafted antibody, a nanobody, a human or humanised antibody or an antigen binding portion of any thereof. For the production of both monoclonal and polyclonal antibodies, the experimental animal is typically a nonhuman mammal such as a goat, rabbit, rat or mouse but may also be raised in other species such as camelids. Polyclonal antibodies may be produced by routine methods such as immunisation of a suitable animal, with the antigen of interest. Blood may be subsequently removed from the animal and the IgG fraction purified. Monoclonal antibodies (mAbs) of the invention can be produced by a variety of techniques, including conventional monoclonal antibody methodology e.g., the standard somatic cell hybridization technique of Kohler and Milstein. The preferred animal system for preparing hybridomas is the murine system. Hybridoma production in the mouse is a very well-established procedure and can be achieved using techniques well known in the art.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

Once a suitable antibody has been identified and selected, the amino acid sequence of the antibody may be identified by methods known in the art. The genes encoding the antibody can be cloned using degenerate primers. The antibody may be recombinantly produced by routine methods.

### Determining the Presence, Absence or Concentration of the Isolated Strain

The invention also provides a method of determining the presence or absence of an isolated strain of the invention. The method of the invention involves detecting the presence or absence of an isolated strain of the invention. In other words, the method involves determining whether or not the isolated strain of the invention is present in the sample. The method may give a positive result, i.e. where the isolated strain of the invention is present in the sample. The method may alternatively give a negative result, i.e. where the isolated strain of the invention is not present in the sample. The method may be helpful to identify diagnose bee colonies as discussed below.

The method comprises contacting the sample with an oligonucleotide of the invention or an antibody of the invention and (b) detecting specific hybridisation of the oligonucleotide or specific binding of the antibody. The presence of specific hybridization or specific binding indicates the presence of the isolated strain in the sample. The absence of specific hybridization or specific binding indicates the absence of the isolated strain in the sample.

The invention also provides a method of determining the concentration of an isolated strain of the invention in a sample, comprising (a) contacting the sample with an oligonucleotide or antibody of the invention and (b) detecting the level of specific hybridisation of the oligonucleotide or specific binding of the antibody and thereby determining the concentration of the isolated strain.

Determining the presence, absence or concentration of the isolated strain allows the detection of the isolated strain of the invention in any bee or bee tissue as described herein. Also, determining the presence, absence or concentration of the isolated strain allows the detection of sequences derived from the strain of the invention, including a polynucleotide and/or oligonucleotide of the invention, that are integrated within the genome of a bee or bee.

The method of determining the concentration of the isolated strain may be used to quantify the isolate strain of the invention by other measures, for example the level, amount or number of viral particles of the isolated strain of the invention.

The sample may be any suitable sample. The invention is typically carried out on a sample that is known to contain or suspected of containing the isolated strain of the invention. The invention may be carried out on a sample that contains one or more viral strains whose identity is unknown. Alternatively, the invention may be carried out on a sample to confirm the identity of the isolated strain of the invention whose presence in the sample is known or expected.

The sample may be a biological sample. The invention may be carried out in vitro on a sample obtained from or extracted from a bee or a bee colony. The sample may be take from any bee tissue. For example, the sample may be taken from tissues from the abdomen, thorax or head of a bee. In particular, a sample may be from brain tissue or gut tissue of a bee. The sample is preferably a fluid sample. Preferably the sample is haemolymph.

The sample may be obtained from a bee at any stage of its lifecycle, including bee eggs, particularly freshly laid eggs, bee larvae, bee pupae and adult bees of any age. The bee may be of any type, including a worker bee, drone bee or queen bee,

The sample to be tested may also be obtained from or extracted from waste material produced during the development of a bee, for example the pupal case that is shed during development, or faeces.

The sample is typically processed prior to being assayed, for example by centrifugation or by passage through a membrane that filters out unwanted molecules or cells. The sample may be measured immediately upon being taken. The sample may also be typically stored prior to assay, preferably below −70° C.

The level of the isolated strain present in a sample encompasses all appropriate measures for quantifying a virus, and includes, for example, the amount, the concentration, the number of virus particles given in any appropriate units.

The presence or level of the isolated strain may be measured direct or indirect means. Any method that allows for the detecting of the isolated strain and the quantification, or relative quantification of the isolated strain may be used. Appropriate technique are known in the art, including, but not limited to, plaque assays, 50% tissue culture infective dose (TCID₅₀) assays, fluorescent focus assays (FFA), protein assays such as the single radial immunodiffusion assay, flow cytometry, quantitative polymerase chain reaction (qPCR) and antibody-based assays.

The level of the isolated strain in a sample of interest may be compared with the level of the isolated strain in another sample. Alternatively, the level of the isolated strain in a sample of interest may be compared with the level of the other DWV strains in the same or another sample.

Any method may be used to detect and quantify specific hybridisation of the oligonucleotide of the invention. The oligonucleotide is typically tagged (or “labelled”) with a molecular marker of either radioactive or fluorescent molecules. Suitable tags (or labels) include, but are not limited to, any of those discussed above, ³²P (a radioactive isotope of phosphorus incorporated into the phosphodiester bond in the probe DNA) an digoxigenin, which is a non-radioactive, antibody-based marker.

Any method may be used to detect and quantify specific binding of the antibody of the invention. Methods of quantitatively measuring the binding of an antibody to an antigen are well known in the art. For example, when an isolated strain of the invention is present in the sample, it may bind or substantially bind with the antibody to form antibody-virus complexes, which may then be detected or quantitatively measured. Detection of such complexes is typically carried out using a secondary antibody which recognises general features in the antibody of the invention. The secondary antibody is typically labelled with a detectable label. This facilitates identification of the autoantibody-antigen complex. Any detectable label may be used. Suitable labels include those discussed above with reference to the polynucleotides of the invention.

For instance, the secondary antibody may be conjugated to an enzyme such as, for example, horseradish peroxidise (HRP), so that detection of an antibody-virus complexes is achieved by addition of an enzyme substrate and subsequent colorimetric, chemiluminescent or fluorescent detection of the enzymatic reaction products, or it may be conjugated to a fluorescent or luminescent signal. Alternatively, the secondary antibody may be labelled with a reporter molecule such as a heavy metal or a radioactive tag. Preferably, the intensity of the signal from the secondary antibody is indicative of the relative amount of the antigen-autoantibody complex in the sample when compared to a positive or negative control, and using different dilutions of the samples.

The binding of antibodies to viruses may be detected by any immunological assay technique, of which many are well known in the art. Examples of suitable techniques include enzyme-linked immunosorbent assays (ELISAs), radioimmunoassays, competition assays, inhibition assays, sandwich assays, fluorescent microscopy, microarrays (such as a protein microarray), fluorescence-activated cell sorting (FACS) or the like.

### Vectors

### Vectors

The polynucleotides and oligonucleotides of the present invention may be provided in the form of a vector which includes a promoter operably linked to the inserted sequence, thus allowing for expression of the polynucleotide or oligonucleotide in a bee cell. Any suitable vector may be used which enables the expression of the polynucleotide or oligonucleotide of the invention.

Accordingly, the present invention thus provides a vector that comprises a polynucleotide or oligonucleotide of the invention. The vector is routinely constructed in the art of molecular biology and may, for example, involve the use of plasmid DNA and appropriate initiators, promoters, enhancers and other elements, such as for example polyadenylation signals which may be necessary, and which are positioned in the correct orientation, in order to allow for expression of the polynucleotide or oligonucleotide of the invention. Other suitable vectors would be apparent to persons skilled in the art. By way of further example in this regard refer to Sambrook et al., 2001, Molecular Cloning: a laboratory manual, 3rd edition, Cold Spring Harbour Laboratory Press; and Current Protocols in Molecular Biology, Ausubel et al., Eds., Greene Publishing and Wiley-Interscience, New York (1995)). Therefore, the vector of the invention may be for example, a plasmid, virus or phage vector provided with an origin of replication, a promoter for the expression of the polynucleotide or oligonucleotide and optionally a regulator of the promoter.

In a preferred embodiment vector is a plasmid vector. A plasmid is an autonomously replicating, extrachromosomal circular or linear polynucleotide. The plasmid may include additional elements, such as an origin of replication, or selector genes. Such elements are known in the art and can be included using standard techniques. Numerous suitable expression plasmids are known in the art. For example, one suitable plasmid is phGFP-S65T (Robinson et al. 2000 Insect Molecular Biology 9(6):625-634), pHCMW-G (Kurita et al. 2004 PANS 101(5): 1263-1267). A plasmid vector of the invention may be modified to include retrotrasposon sequences from bees or related insects, for example from Drosophila. Such retrotransposons are known in the art, with 60-450 such sequences being known in honeybees. Any functional retrotrasnposon sequence may be used, including, but not limited to, the mariner element known in the art.

In one embodiment, the plasmid vector of the invention is a bacterial plasmid, i.e. a DNA plasmid comprising T7 promoters operably linked to the polynucleotide or oligonucleotide of the invention. The T7 promoters allow the expression of dsRNA molecules from the polynucleotide or oligonucleotide of the invention, which are useful in generating an RNAi immune response as described herein.

In another preferred embodiment, the vector of the invention is a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors. In the case of viral vectors, administration of the polynucleotide or oligonucleotide is mediated by viral infection of a bee cell.

For example, the polynucleotide or oligonucleotide can be inserted into a viral vector and packaged as retroviral particles using techniques known in the art. The isolated strain of the invention may be produced synthetically be inserted the polynucleotide of the invention into a suitable viral vector or viral envelope. Production of the isolated strain can be carried out be introducing the virus particle into an appropriate host cell in which the virus can replicate. This can be carried out using standard techniques known in the art. The virus particles can then be isolated and/or concentrated using known techniques.

The recombinant virus comprising the polynucleotide or oligonucleotide of the invention can then be isolated and delivered to a bee cell. Preferably the virus is delivered to a bee cell, most preferably a bee germ cell. The virus may be delivered either in vivo or ex vivo, preferably in vitro.

Viral vectors may be based on any suitable virus. For example, retroviral vectors may be based upon the Moloney murine leukaemia virus (Mo-MLV) or human-immunodeficiency virus (HIV). A number of adenovirus vectors are known. Adenovirus subgroup C serotypes 2 and 5 are commonly used as vectors. The wild type adenovirus genome is approximately 35 kb of which up to 30 kb can be replaced with foreign DNA. Suitable adenoviral vectors include Ad5 vectors and simian adenovirus vectors. Viral vectors may also be derived from the pox family of viruses, including vaccinia viruses and avian poxvirus such as fowlpox vaccines. Additional types of virus such as adeno-associated virus (AAV) and herpes simplex virus (HSV) may also be used to develop suitable vector systems.

In a preferred embodiment, the polynucleotide, oligonucleotide or vector of the invention is encapsulated in a viral envelope. In a most preferred embodiment, the viral envelope is a vesicular stomatitis virus envelope (pseudotype virus).

Methods for the production of viral vectors are well known in the art. One non-limiting example of such an approach is a viral vector comprising three separate plasmids: (1) an envelope vector comprising a plasmid encoding a viral coat (capsid) or envelope operably linked to a promoter; (2) a plasmid encoding the non-structural viral genes required for viral replication operably linked to a promoter (the so-called “packaging” vector); and (3) a transducing vector comprising a plasmid in which the sequence of interest operably linked to a promoter.

One such viral vector is described in Coleman et al. (Physiol. Genomics (2002) 12: 221-228). Colmen et al. discloses a viral vector comprising three separate plasmids: (1) the pHEF-VSVG plasmid which encodes the Vesicular Stomatitis Virus (VSV) glycoprotein under the control of an EF1 promoter (commercially available from Addgene, http://www.addgene.org/22501/); (2) the pNHP plasmid which encodes lentiviral gag and pol proteins (commercially available from Addgene, http://www.addgene.org/22500/); and (3) a pTYF-mCMV/SYN-EGFP which encodes a green fluorescent protein under the control of a cytomegalovirus promoter (commercially available from Addgene, http://www.addgene.org/199751).

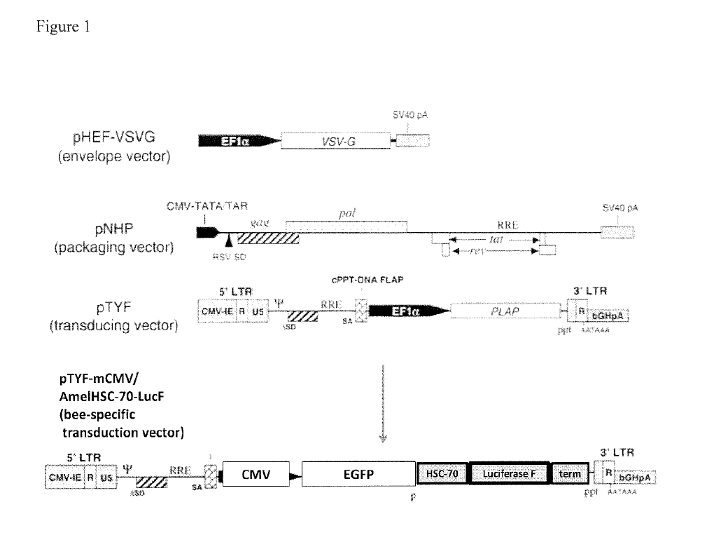

In the present invention, the transducing vector comprises a polynucleotide or oligonucleotide of the invention. The transducing vector may be derived from any suitable vector, such as the pTYF-mCMV/SYN-EGFP of Colmen et al. to comprise a polynucleotide or oligonucleotide of the invention under the control of a suitable promoter, such as the particular promoters described below. FIG. 1 illustrates schematically a preferred envelope vector, packaging vector and transducing vector of the invention. The transducing vector of the invention may be modified to replace the luciferase reporter gene with a polynucleotide or oligonucleotide of the invention. In a preferred embodiment, the transducing vector of the invention may be derived from SEQ ID NO: 7 or 73, into which is inserted a payload sequence operably linked to a suitable promoter, such as those described herein.

As an alternative to viral vectors, liposomal preparations can alternatively be used to deliver the polynucleotide or oligonucleotide of the invention. Useful liposomal preparations include cationic (positively charged), anionic (negatively charged) and neutral preparations, with cationic liposomes particularly preferred. Cationic liposomes may mediate intracellular delivery of the polynucleotide or oligonucleotide.

As another alternative to viral vector systems, the polynucleotide or oligonucleotide may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyornithine, spermine, spermidine, as well as conjugates of these molecules.

The vector of the invention comprises the polynucleotide or oligonucleotide operably linked to a promoter. Operably linked refers to an arrangement of elements wherein the components so described are configured so as to perform their usual function. Thus, a promoter operably linked to the polynucleotide or oligonucleotide sequence is capable of effecting the expression of that sequence when the proper enzymes are present. Promoters for use in the present invention are discussed further below.

In addition to the polynucleotide or oligonucleotide sequence operably linked to a promoter, additional sequences may be present. These additional sequences are discussed further below.

The vector may be used in vitro, for example to transfect or transform a bee cell to produce a bee cell comprising the vector of the invention. Preferably a vector comprising the oligonucleotide of the invention enables incorporation of the oligonucleotide into the genome of the bee cell. Preferably the bee cell is a bee germ cell. The vectors may also be adapted to be used in vivo, for example to allow in vivo expression of the polynucleotide or oligonucleotide. This is discussed in more detail below.

The polynucleotide, oligonucleotide or vector of the present invention may be administered directly in a “naked” form, meaning that they are not carried in any delivery vehicle. Alternatively, as discussed below, the polynucleotide, oligonucleotide or vector may be administered in a composition comprising a delivery vehicle, such as a sugar syrup.

### Promoters

A “promoter” is a nucleotide sequence which initiates and regulates transcription of a polypeptide-encoding polynucleotide. Promoters can include inducible promoters (where expression of a polynucleotide or oligonucleotide sequence operably linked to the promoter is induced by an analyte, cofactor, regulatory protein, etc.), repressible promoters (where expression of a polynucleotide sequence or oligonucleotide operably linked to the promoter is repressed by an analyte, cofactor, regulatory protein, etc.), and constitutive promoters. It is intended that the term “promoter” includes full-length promoter regions and functional (e.g., controls transcription or translation) segments of these regions.

Promoters for use in the invention are selected to be compatible with the bee cell for which expression is designed. In a preferred embodiment, the promoter is the honeybee heat shock protein 70 (hsp70) promoter. In a most preferred embodiment the honeybee hsp70 promoter comprises the sequence of SEQ ID NO: 8. The honeybee hsp70 promoter is located at nucleotide positions 7306390 to 7307483 of the honeybee genome (Genbank Accession No. NC_{—}007070, Version No. NC_{—}007070.3 GI:323388987). The present inventors have demonstrated that the honeybee hsp70 promoter is function using experiments in bumblebees (Bombus mori), by attaching the promoter in front of a luciferase reporter gene and demonstrating expression of the luciferase reporter gene upon injection of bumblebees with the construct.

The promoter need not be contiguous with the polynucleotide or oligonucleotide sequence, so long as it functions to direct the expression thereof. Thus, for example, intervening untranslated yet transcribed sequences can be present between the promoter sequence and the polynucleotide or oligonucleotide sequence and the promoter sequence can still be considered “operably linked” to the polynucleotide or oligonucleotide sequence.

### Additional Sequences

The vector of the invention may comprise one or more additional sequence in addition to the polynucleotide/oligonucleotide and promoter of the invention. These additional sequences may regulate the expression of the polynucleotide or oligonucleotide.

A polynucleotide, oligonucleotide or vector of the invention may comprise an untranslated leader sequence. In general the untranslated leader sequence has a length of from about 10 to about 200 nucleotides, for example from about 15 to 150 nucleotides, preferably 15 to about 130 nucleotides. Leader sequences comprising, for example, 15, 50, 75 or 100 nucleotides may be used. Generally a functional untranslated leader sequence is one which is able to provide a translational start site for expression of a coding sequence in operable linkage with the leader sequence.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra. In a preferred embodiment, a vector of the invention comprises a honeybee transcription terminator sequence of SEQ ID NO: 9. This honeybee transcription terminator is located at nucleotide positions 7311340 to 7311727 of the honeybee genome (Genbank Accession No. NC_{—}007070, Version No. NC_{—}007070.3 GI:323388987).

The polynucleotide, oligonucleotide vector may include transcriptional modulator elements, referred to as “enhancers”. Enhancers are broadly defined as a cis-acting agent, which when operably linked to a promoter and polynucleotide/oligonucleotide sequence, will increase transcription of that polynucleotide or oligonucleotide sequence. Enhancers can function from positions that are much further away from the polynucleotide or oligonucleotide sequence of interest than other expression control elements (e.g. promoters), and may operate when positioned in either orientation relative to the sequence of interest. Enhancers have been identified from a number of viral sources, including polyoma virus, BK virus, cytomegalovirus (CMV), adenovirus, simian virus 40 (SV40), Moloney sarcoma virus, bovine papilloma virus and Rous sarcoma virus. Examples of suitable enhancers include the SV40 early gene enhancer, the enhancer/promoter derived from the long terminal repeat (LTR) of the Rous Sarcoma Virus, and elements derived from human or murine CMV, for example, elements included in the CMV intron A sequence.

The vector may contain one or more selectable marker genes, for example an ampicillin resistance gene in the case of a bacterial plasmid or a resistance gene for a fungal vector.

The vector may include a retroviral polynucleotide or oligonucleotide which facilitate the insertion of the oligonucleotide of the invention into the bee genome. Typically plasmid vectors of the invention include such retroviral sequences.

### Inhibition of Deformed Wing Virus

As discussed above, the inventors have identified a single strain of DWV, the isolated strain of the invention, that is predominant in bees and bee colonies infested with Varroa mites and which is present at high levels in the infested bees and colonies.

Therefore, inhibitors of the isolated strain of the invention may be used to treat and/or prevent infection by the strain, and so reduce the impact of infection on the bee or bee colony. Inhibitors of the isolated strain of the invention may also be used to treat and/or prevent deformed wing in a bee or bee colony infested with Varroa mites.

Accordingly, the present invention provides methods and compositions for inhibiting the isolated strain of the invention. The inhibitors for use in the invention are able to reduce the level, amount or concentration of the isolated strain within a bee. The compositions and methods of the invention can be used in individual cells, cells or tissue in culture, or in vivo in bees, or in organs or other portions of bees. The compositions and methods of the invention can be used to treat part of or entire bee colonies.

Inhibition of the isolated strain of the invention may be measured by any suitable means. For example, the Examples herein disclose methods for determining infection or viral load. Infection or viral load may be used as an indicator of the effectiveness of the inhibitors of the invention: the more effective an inhibitor, the greater the decrease in infection or viral load.

According to the present invention, inhibitors of the isolated strain of the invention can be used for reducing the presence and/or level of viral particles in a bee cell, bee or bee colony. The inhibitors may achieve this reduction by reducing the expression of or cause silencing or knockdown of any gene of the isolated strain of the invention. The inhibitors of the invention may cause reduction of expression of at least one viral protein. Typically, the reduction in level of viral particles in the cell, bee or bee colony may be at least 50% of the amount of viral particles in the absence of the inhibitor. Preferably, the reduction is at least 55%, at least 60%, at least 65%, at least 70%, at least 75% or at least 80% or more preferably, at least 85%, at least 90% or at least 95%. A method for determining the relative amount of viral particles may be any suitable method known in the art.

### Inhibitors

Any suitable inhibitor may be used according to the invention, for example peptides and peptidomimetics, antibodies, small molecule inhibitors, double-stranded and antisense RNA, aptamers and ribozymes. Preferred inhibitors are polynucleotide or oligonucleotide inhibitors, more preferably oligonucleotide inhibitors, and most preferably RNA oligonucleotide inhibitors.

Inhibitors for use in the invention include the antibodies, polynucleotides, oligonucleotides, vectors, and compositions of the invention. The most preferred inhibitors are the polynucleotides and oligonucleotides of the invention.

The inhibitors of the invention may be delivered to a bee or bee cell by any appropriate means. In particular, the polynucleotides and oligonucleotides of the invention may be delivered as naked sequences, preferably in the form of dsRNA or hairpin RNA structures, as DNA molecules operably linked to a suitable promoter, in a vector, particularly a plasmid or viral vector as discussed herein.

### Pharmaceutical Compositions

In some embodiments, the polynucleotide, oligonucleotide, antibody or vector of the invention is contained in a composition comprising a delivery vehicle. The delivery vehicle is preferably a sugar syrup. This allows the composition to be fed to a target bee and allows uptake of the molecule or inhibitor by the target bee. In a preferred embodiment the sugar syrup comprises a 1:1 w/v mix of granulated sugar and water, a 2:1 w/v mix of granulated sugar and water, invertase treated sugar or sugar solutions. Fondant, particularly bakers fondant may also be used a delivery vehicle. Alternatively, or in addition to these sugars-based delivery vehicles, real or artificial pollen patties may be used.

The formulated compositions will include an appropriate amount of the polynucleotide, oligonucleotide, antibody or vector of the invention which is sufficient achieve the desired result. For example, the composition may comprise an amount of the oligonucleotide of the invention that is sufficient to generate an RNAi immune response against the isolated strain of the invention when the composition is contacted with a bee. An appropriate effective amount can be readily determined by one of skill in the art. Such an amount will fall in a relatively broad range that can be determined through routine trials. The compositions may can be administered directly to a bee or, alternatively, delivered ex vivo, to bee cells, using methods known to those skilled in the art.

### Bees

The invention concerns treating and diagnosing bees. The bee may be any type of bee, including, without limitation, honeybees (of the genus Apis) and bumblebees (of the genus Bombus). Preferably the bee is Apis mellifera, Apis cerana, Bombus terrestris or Bombus impatiens. More preferably the bee is Apis mellifera. The term “bee” as used herein covers all stages of the bee life cycle. Thus, egg, larval, pupal and adult bees are all covered by the term “bee”.

Bee cells, including bee germ cells, may also be used according to the present invention. A bee germ cell is a cell that is responsible for transferring the bee genome to the next generation of bee. Examples include bee gametes, such as bee spermatozoa or bee eggs (oocytes).

The present invention may also be applied to a bee colony. A bee colony typically comprises at least one queen bee and two or more, preferably many, worker and/or drone bees. Preferably the bee colony also comprises at least one bee egg and/or at least one bee larva. The bee colony may also comprise at least one bee pupa.

### Delivery Regimes

The compositions are administered to a bee in an amount that is compatible with the dosage formulation and that will be prophylactically and/or therapeutically effective. An appropriate effective amount will fall in a relatively broad range but can be readily determined by one of skill in the art by routine trials.

As used herein, the term “prophylactically or therapeutically effective dose” typically means a dose in an amount sufficient to reduce the level of an isolated strain of the invention as discussed above. The term “prophylactically or therapeutically effective dose” may mean a dose in an amount sufficient to alleviate, reduce, cure or at least partially arrest symptoms and/or complications from infection with an isolated strain of the invention. The term “prophylactically or therapeutically effective dose” may mean a dose in an amount sufficient to alleviate, reduce, cure or at least partially arrest symptoms and/or complications from Varroa mite-induced deformed wing.

Prophylaxis or therapy can be accomplished by a single direct administration at a single time point or by multiple administrations, optionally at multiple time points. Administration can also be delivered to a single or to multiple sites within a bee. Further, administration may be to an individual bee or to more than one bee. Administration may be to the whole or part of one or more bee colony. Those skilled in the art can adjust the dosage and concentration to suit the particular route of delivery. In one embodiment, a single dose is administered to a bee or a bee colony on a single occasion. In one embodiment, multiple doses are administered to a bee or bee colony on multiple occasions. Any combination of such administration regimes may be used.

Different administrations may be performed on the same occasion, on the same day, one, two, three, four, five or six days apart, one, two, three, four or more weeks apart. Preferably, administrations are 1 to 5 weeks apart, more preferably 2 to 4 weeks apart, such as 2 weeks, 3 weeks or 4 weeks apart. The schedule and timing of such multiple administrations can be optimised for a particular composition or compositions by one of skill in the art by routine trials.

For the administration of inhibitors of the invention, particularly exogenous polynucleotides or oligonucleotides of the invention (for the generation of an RNAi immune response) via a feeding delivery route, the preferred times for delivery are early spring, typically March or April in the UK, or late summer/early autumn, typically August or September in the UK. Bee keepers typically feed bees at both these times, such that either solid or liquid food may be used as a delivery vehicle as described herein. Typically bee keepers feed bees for three to six weeks within these time periods. The feeding periods are weather-dependent and may be altered as necessary according to the specific conditions.

Further, administration may take place at any other time a bee keeper routinely feeds the bee. For example, bee keepers typically feed bees after hiving a swarm. Therefore, an inhibitor of the invention may be administered on feeding after hiving a swarm. This is a particularly preferred time for administration, because it would allow the treatment and/or prevention of the isolated strain of the invention or Varroa mite-induced deformed wing disease according to the invention in a newly formed bee colony.

Dosages for administration will depend upon a number of factors including the nature of the composition, the route of administration and the schedule and timing of the administration regime. The dose will also vary according to the severity of the infection with the isolated strain and/or the deformed wing in a Varroa mite-infested bee or bee colony. The dose will also vary according to number of bees or bee colonies to be treated. Optimum dosages may vary depending on the relative potency of the oligonucleotide or antibody of the invention, and can generally be estimated based on EC50s found to be effective in vitro and in in vivo animal models.

### Therapeutic Applications

As discussed above, the isolated strain of the invention has been identified at high concentrations and with low diversity in bee colonies infested with Varroa mites. DWV infection and Varroa infestation are coincident with the appearance of pathology in the bees, including the appearance of wing deformities. In contrast, bee colonies not infested with Varroa mites have low-concentration, covert DWV infections with a high diversity of DWV strains.

Accordingly, the present invention provides a method of treating or preventing in a bee or bee colony infection with the isolated strain of the invention, comprising contacting the bee or bee colony with an inhibitor of the isolated strain. The invention also provides an inhibitor of the isolated strain of the invention for use in a method of treating and/or preventing in a bee or bee colony infection with the isolated strain, comprising contacting the bee or bee colony with an inhibitor of the isolated strain. The invention also provides use of an inhibitor of the isolated strain of the invention in the manufacture of a medicament for treating and/or preventing in a bee or bee colony infection with the isolated strain

The invention also provides a method of treating or preventing deformed wing disease in a Varroa mite-infested bee or bee colony infection, comprising contacting the bee or bee colony with an inhibitor of the isolated strain. The invention also provides an inhibitor of the isolated strain of the invention for use in a method of treating and/or preventing deformed wing disease in a Varroa mite-infested bee or bee colony, comprising contacting the bee or bee colony with an inhibitor of the isolated strain of the invention. The invention also provides use of an inhibitor of the isolated strain of the invention in the manufacture of a medicament for treating and/or preventing deformed wing disease in a Varroa mite-infested bee or bee colony.

In a preferred embodiment the inhibitor is specific for the isolated strain of the invention. An inhibitor is specific for the isolated strain of the invention if is inhibits the isolated strain of the invention, but inhibits other viruses, such as other strains of DWV, to a lower degree or not at all. In a more preferred embodiment, the inhibitor is a polynucleotide of the invention, an oligonucleotide of the invention, an antibody of the invention, a vector of the invention or a composition of the invention.

The therapeutic or prophylactic method of the invention may comprise contacting at least one bee with an oligonucleotide of the invention to stimulate the bee's immune system to generate resistance to the isolated strain of the invention.

### Inhibition of DWV by RNAi

The methods of the invention preferably involve generating bees that are resistant to the isolated strain of the invention by contacting at least one bee with a polynucleotide or an oligonucleotide of the invention and thereby stimulating the at least one bee's immune system to generate resistance to the isolates strain. In a preferred embodiment, the polynucleotide or oligonucleotide is fed to the at least one bee. Typically the polynucleotide or oligonucleotide is operably linked to a promoter, contained in a vector and encapsulated in a viral envelop (preferably from vesicular stomatitis virus) to form a pseudotyped retrovirus particle. The pseudotyped retrovirus particles are then typically added to a sugar syrup, facilitating their ingestion by the at least one bee. Adult bees and larval bees will ingest the syrup.

Alternatively, the pseudotyped retrovirus particles may be mixed with drone semen and used during instrumental insemination of virgin bee queens to produce transgenic fertilised bee queens that are capable of transmitting the RNAi-expressing DNA vertically to progeny bees.

Alternatively, a non-integrating or integrating plasmid vector of the invention capable of being maintained and vertically transmitted may be introduced to drone semen for use in instrumental insemination.

The at least one bee is contacted with a polynucleotide or an oligonucleotide of the invention, either in its naked form or comprised in a vector or composition of the invention. On contact, the bee will ingest the polynucleotide or oligonucleotide. After ingestion, the polynucleotide or oligonucleotide is taken up by cells of the at least one bee. Once inside a bee cell, the polynucleotide or oligonucleotide is processed by the bee's intracellular machinery to generate an RNAi immune response. In particular, the polynucleotide or oligonucleotide may be expressed in the bee cell in a hairpin configuration. These hairpin sequences do not integrate into the bee genome, but may be processed by the endogenous enzyme (Dicer), and associate with Argonaute protein (Ago). Dicer and Ago are involved in the early stages of RNAi processing and expression. Dicer cleaves the dsRNA into siRNAs. Ago directs the resulting siRNAs protein complex, the RNA-induced silencing complex (RISC), from the bee cell to complementary RNA molecules from the isolated strain within the bee cell. Contact of the RISC to the viral RNAs targets the viral RNAs for degradation using cellular ribonucleases or inhibits translation of the RNA. Thus, the ability of the isolated strain to replicate in the bee cell is reduced. Consequently, the bee itself is resistant to the isolated strain.

Entry of polynucleotides or oligonucleotides delivered in a viral vector of the invention to a bee cell is mediated by the viral coat or capsid, which is preferably the VSV capsid as described herein. Once the bee cell is infected with the viral vector, the viral vector migrates to the bee cell nucleus and integrates. The vector payload, i.e. the polynucleotide or oligonucleotide of the invention operably linked to a suitable promoter is then expressed in a form that generates dsRNA molecules. These dsRNA molecules are processed by Dicer, leading to the Ago and RISC-mediated cascade described above.

Expression of one or more oligonucleotides of the invention as hairpin sequences allows a highly focused means of suppressing DWV replication. Alternatively, multiple oligonucleotides of the invention or a more extensive region of the polynucleotide of the invention may be expressed, enabling the generation of longer dsRNA for Dicer-mediated processing. Such an embodiment may be used to reduce the risk of resistant forms of DWV emerging, because a greater number of RNAs from the isolated strain can be targeted by the bee protein complexes. Such multiple oligonucleotides or more extensive polynucleotides may be expressed with two opposing promoters, preferably honeybee promoters.

### Transgenic Bees

The present invention also provides a transgenic bee that is resistant to the isolated strain of the invention. The transgenic bee may be an adult bee or a larval bee. Typically the bee is engineered to express an oligonucleotide of the invention, making the bee resistant to the isolated strain of the invention. Typically the transgenic bee comprises at least one cell that has been modified in a way that confers resistance to the isolated strain of the invention. The transgenic bee may be modified such that at least one cell expresses at least one oligonucleotide of the invention. The transgenic bee may be modified such that an oligonucleotide or polynucleotide of the invention has been incorporated into the genome of at least one cell, such that at least one oligonucleotide of the invention is synthesised within the at least one cell. The invention also provides a transgenic bee that is resistant to Varroa mite-induced deformed wing disease, wherein at least one cell of the bee has been modified in a way that confers resistance to deformed wing disease. The transgenic bee may be modified such that at least one cell expresses at least one oligonucleotide of the invention. The transgenic bee may be modified such that an oligonucleotide or polynucleotide of the invention has been incorporated into the genome of at least one cell, such that at least one oligonucleotide of the invention is synthesised within the at least one cell.

Typically a sufficient number of bee cells have been modified as described above such that the bee is resistant to the isolated strain or to deformed wing disease. The polynucleotide or at least one oligonucleotide may be stably incorporated into the bee genome. Alternatively, the polynucleotide or at least one oligonucleotide, or the vector, particularly viral or plasmid vector, containing the polynucleotide or at least one oligonucleotide may be retained within the bee cell(s) without incorporation into the genome.

In a preferred embodiment, the transgenic bee of the invention is a queen bee. The queen bee may be an adult queen bee or a larval queen bee. A transgenic queen bee allows the production of a bee colony which is resistant to the isolated strain of the invention.

The invention also provides one or more bee germ cells which expresses at least one oligonucleotide of the invention. Preferably the oligonucleotide is incorporated into the genome of the bee germ cell(s). The one or more bee germ cell may comprise a polynucleotide of the invention incorporated into the genome of at the bee germ cell(s), such that at least one oligonucleotide of the invention is synthesised within the at least one cell. Thus, the invention provides one or more bee germ cells comprising an oligonucleotide, poly nucleotide or vector of the invention. The one or more bee germ cells are preferably one or more bee spermatozoa.

The transgenic bee of the invention may be designed to express regions of the genome of the isolated strain of the invention, such a way that they induce small interfering RNA (siRNA or RNAi) molecules. In a preferred embodiment, these oligonucleotides are engineered into the genome of the bee, such that the resulting resistance to the isolate strain is stable and may be passed on to subsequent generations.

Bees can be considered a superorganism with a single fertilized queen, several thousand female (unfertilized) workers and a small number of male (fertile) drones within a single colony. Queens mate within 2 weeks of emergence and never leave the colony (other than when swarming). Workers cannot mate and only under exceptional circumstances lay unfertilized eggs (which develop into drones). Fertilized eggs develop into queens or workers depending upon how they are maintained in the first 3 days after hatching. Beekeepers have developed ways to raise queens from any newly hatched larvae.

To introduce polynucleotides or oligonucleotides of the invention into the germline of bees it is necessary to manipulate a queen bee to lay fertilized eggs containing the polynucleotides or oligonucleotides. Sperm mediated gene transfer (SMGT) in honeybees is known in the art (Robinson et al. 2000 Insect Molecular Biology 9(6): 625-634).

Accordingly, the invention provides a method of generating a transgenic queen bee that is resistant to infection by an isolated strain of the invention, comprising (a) incorporating a polynucleotide or an oligonucleotide of the invention into the genome of one or more bee germ cells, such as one or more spermatozoa; and (b) generating the queen bee from said one or more germ cells. This method may also be used to generate a transgenic queen bee that is resistant to Varroa mite-induced deformed wing disease. Bee spermatozoa may be collected from one or more drone bee.

The polynucleotide or oligonucleotide of the invention may be contacted with the bee germ cell using methods known in the art (Robinson et al 2000), such that the polynucleotide or oligonucleotide is incorporated into the genome of the bee germ cell. The polynucleotide or oligonucleotide may be contacted with the bee germ cell as a naked polynucleotide or oligonucleotide, or in a vector or composition of the invention. In particular, the polynucleotide or oligonucleotide may be incorporated into the bee germ cell using retroviral mediated infection. The retroviral vector used in said method may be as described herein.

The bee germ cell, particularly the bee spermatozoa, comprising the polynucleotide or oligonucleotide of the invention may then be transferred to a virgin queen bee. In the case of bee spermatozoa, the spermatozoa will migrate to the spermatheca where they are stored for months to years and can be used to fertilise the eggs of the virgin queen bee to generate a transgenic bee. Preferably the transgenic bee is a queen bee.

Once the polynucleotide or oligonucleotide of the invention has been contacted with the bee germ cell it will first be taken up by that cell. After uptake of the polynucleotide or oligonucleotide by the bee germ cell, the polynucleotide or oligonucleotide will be incorporated into the genome of the bee germ cell. This can be mediated by retroviral sequences provided in a vector of the invention as discussed above. Incorporation of the polynucleotide or oligonucleotide into the genome allows the oligonucleotide to be transmitted vertically to a progeny bee, thus enabling the production of bee that are resistant to the isolated strain of the invention or to Varroa-mite induced deformed wing.

As discussed above, the polynucleotides and oligonucleotides of the invention may be useful in generating an small interfering RNA (siRNA or RNAi) immune response against the isolated strain of the invention. As a result of this RNAi immune response, the transgenic bee will be resistant to the isolated strain of the invention or to Varroa-mite induced deformed wing disease. In a preferred embodiment, the polynucleotide or oligonucleotide is reverse-transcribed in a bee cell, resulting in DNA forms embedded in retrotransposon sequences to generate the RNAi immune response.

Alternatively, transgenic bees of the invention may be generated by other suitable techniques, such as feeding developing drones with the retroviral vectors of the invention, or microinjecting developing drone larvae or pupae with the retroviral vectors of the invention.

Once a transgenic queen bee of the invention has been produced, this transgenic queen can be used produce transgenic progeny bees that also comprise the oligonucleotide of the invention and so are resistant to the isolated strain of the invention or to Varroa-mite induced deformed wing disease. The transgenic queen bee of the invention can be used to initiate new a new bee colony, the worker and drone bees of which will all transgenic and hence resistant to the isolated strain of the invention or to Varroa-mite induced deformed wing disease. Accordingly, the invention also provides a method of generating a transgenic bee of the invention, comprising producing the transgenic bee from a transgenic queen bee of the invention or from a transgenic queen bee producing using a method of the invention.

The invention further provides a method of preventing in a bee or bee colony an infection with an isolated strain of the invention, comprising using a transgenic queen bee of the invention or from a transgenic queen bee produced using a method of the invention to generate the bee or the bee colony.

The invention further provides a method of preventing deformed wing disease in a Varroa mite-infested bee or bee colony, comprising using a transgenic queen bee of the invention or from a transgenic queen bee produced using a method of the invention to generate the bee or bee colony.

The invention further provides a method of producing a bee or bee colony that is resistant to infection from an isolated strain the invention, comprising using a transgenic queen bee of the invention or from a transgenic queen bee produced using a method of the invention to generate the bee or bee colony.

### Resistant Bee Colonies

As discussed herein, the inventors have identified a sub-population of bees present in Varroa mite-infested colonies that have sequences from the strain of the present invention integrated into their genomes. Such bees are naturally resistant to the isolated strain. However, whilst a sub-population of bees in Varroa mite-infested colonies might be naturally resistant to the isolated strain, the presence of such a naturally resistant sub-population is not sufficient for colony survival. In practice, a bee colony will only survive if a sufficient number of individual bees are resistant to the isolated strain.

Bees comprising such incorporated sequences may be identified according to the present invention, for example using the oligonucleotides or antibodies of the invention in the methods of determining the presence or absence of the isolated strain of the invention. These naturally resistant bees may be used to generate bees or bee colonies that are also resistant to the isolated strain of the invention, or that are resistant to Varroa mite-induced deformed wing disease.

The invention provides for the first time a bee colony comprising sufficient bees that are individually resistant to infection by the isolated strain of the invention, such that the colony as a whole is resistant infection by the isolated strain or to Varroa mite-induced deformed wing disease. Typically at least 50%, at least 60%, at least 70%, at least 80, at least 90% or more, up to 100% of the individual bees within a colony must be resistant to the isolated strain in order for the colony to be resistant to the isolated strain or to Varroa mite-induced deformed wing disease.

Accordingly, the invention provides a bee colony wherein at least 50% of the bees within the colony are resistant to the isolated strain of the invention. The invention also provides a bee colony wherein at least 50% of the bees within the colony are resistant to Varroa mite-induced deformed wing disease. Typically the at least 50% of the bees comprise an oligonucleotide of the invention, a polynucleotide of the invention comprising two or more oligonucleotides of the invention, or a vector of the invention. In a preferred embodiment, the individual bees that are resistant to the isolated strain of the invention have an oligonucleotide of the invention or a polynucleotide of the invention comprising two or more oligonucleotides of the invention incorporated into their genome.

The present invention also provides a method of producing a bee or bee colony that is resistant to the isolated strain of the invention comprising: identifying a bee or bee larva that have sequences from the strain of the invention incorporated into the bee or bee larva genome; generating a queen bee from said bee or bee larva; and using said queen bee to generate the bee or bee colony. The present invention also provides a method of producing a bee or bee colony that is resistant to Varroa mite-induced deformed wing disease comprising: identifying a bee or bee larva that have sequences from the strain of the invention incorporated into the bee or bee larva genome; generating a queen bee from said bee or bee larva; and using said queen bee to generate the bee or bee colony.

### Diagnostic Applications

The invention provides a method of diagnosing in a bee or bee colony infection with an isolated strain of the invention, comprising determining the presence or absence of the isolated strain, wherein the presence of the isolated strain is indicative of the presence of infection with the isolated strain and wherein the absence of the isolated strain is indicative of the absence of infection with the isolated strain. Typically the determination step takes place in vitro in sample from the bee or colony. Suitable samples are discussed above.

The invention also provides a method of diagnosing deformed wing disease in a Varroa mite-infested bee or bee colony, comprising determining the presence or absence of an isolated strain of the invention, wherein the presence of the isolated strain is indicative of the presence of deformed wing disease and wherein the absence of the isolated strain is indicative of the absence of deformed wing disease. Typically the determination step takes place in vitro in sample from the bee or colony. Suitable samples are discussed above.

In a preferred embodiment, an oligonucleotide of the invention or an antibody of the invention is used to identify the presence or absence of the isolated strain as described above.

Methods of determining the presence or absence of the isolated strain of the invention and methods of determining the level of the isolated strain as disclosed herein may be used in the diagnosis in a bee or bee colony of infection with the isolated strain of the invention or the diagnosis of deformed wing disease in a Varroa mite-infested bee or bee colony.

The invention is illustrated by the following Examples:

### EXAMPLES

### Introduction

There are indications that in the absence of the Varroa mite DWV is transmitted vertically through eggs and male sperm. This route of transmission favours covert infection with low pathogenicity and DWV levels, because it depends on the survival of the host. Moreover, it cannot be ruled out that DWV may be associated with favourable bee traits. For example KV (a Japanese strain of DWV) is associated with aggressive behaviour traits, which could be beneficial for the colony survival. With the spread of Varroa destructor mites, which reached the UK in the 1990s, overt DWV infections became common. There is a possibility that pathogenicity of DWV increased in Varroa-infested colonies due to changes in DWV populations facilitated by the mite. The Varroa mite, which is a host to at least some DWV variants, provides a route for effective horizontal transmission of DWV, therefore the virus was no longer dependent on the survival of infected individuals. Moreover, aggressive strains of DWV accumulating to higher levels faster could be more easily acquired from infected bees and transmitted by the mite. Changes in DWV population composition on a large temporal and spatial scale following Varroa infestation were reported for Hawaiian honeybees, although this study did not provide enough DWV sequence data to pinpoint the identity of the winning virus strains. There is no agreement on whether the feeding by Varroa mites suppresses bee innate immunity, which would make possible the replication of DWV to high levels. Earlier reports showed that bees exposed to Varroa feeding are immuno-compromised, but later transcriptome analysis studies found little or no evidence of negative effects of Varroa on the expression of innate immunity genes. It should be noted, however, that analysis of the impact of Varroa mite feeding on antivirus defences in bees is difficult, because these defences remain poorly understood in all insects, including the much better studied Drosophila and mosquito. Although RNA interference (RNAi) is considered to be the major defence against RNA viruses in insects, there is evidence suggesting that Toll, Imd and Jak-Stat signalling pathways are also involved in antivirus resistance. It is possible that Varroa destructor interferes with antivirus pathways in bees and that contributes to activation of DWV.

The present inventors sought to explore both hypotheses on the role of Varroa in dramatic increase of DWV pathogenicity and the replication levels in bees, namely: (i) Varroa infestation results in the introduction of pathogenic strains of DWV, which were selected for their ability to be effectively transmitted by Varroa mites, or (ii) Varroa mites disrupt bee antivirus defence mechanisms, including immune defence pathways, RNAi machinery, or tissue barriers. To test these distinct, but not mutually exclusive hypotheses, the inventors devised a system which modelled encounter of naïve Varroa-free bees with Varroa mites and the mite-associated DWV strains. We infected bees from a Varroa-free honeybee colony with the DWV strains circulating in a Varroa-infested colony, either solely orally or orally and via injection into bee haemolymph by the mites. The inventors analysed the virus population dynamics, RNAi responses, and the global gene expression in the bees subjected to the above treatments and in the control bees. Analysis of the resulting changes in DWV populations and in the bee transcriptome provided data indicating that both introduction of pathogenic strains of DWV and suppression of antivirus defences play their parts in activation of DWV replication in Varroa-infested bees. The results suggest that pathogenic strains of DWV are effectively suppressed when delivered orally, although oral infection results in changes in DWV diversity. Varroa mite feeding on developing pupae, which allowed direct injection of pathogenic DWV strains into haemolymph as well as suppression of innate immunity, were the likely causes of DWV activation in the mite associated pupae.

### Example 1

### Experimental Infestation of Bees with Varroa Mites and Varroa-Associated DWV Resulted in Overt and Covert DWV Infection

Frame Transfer System for Honeybee Infestation with DWV and Varroa Mites

Infestation of bees included transfer of a brood frame with newly hatched larvae from Varroa-free colony to Varroa-infested colony and subsequent sampling the capped pupae. A Varroa-free colony of black British honeybee, Apis mellifera mellifera with a naturally-mated one year old queen was imported from Colonsay, Scotland, an island with no historic reports of Varroa incidence and no imports of bees from Varroa-infested areas. The presence of DWV strains associated with Varroa mite infestation could thus be excluded. As a source of Varroa mites and the mite-associated DWV strains, a Warwickshire honeybee colony, heavily infested with Varroa and having high DWV levels in bees and mites was selected. The Varroa-free and Varroa-infested colonies were contained in separate mesh flight cages and were maintained on an artificial diet of sugar syrup and pollen. The pollen was imported from Varroa-free Australia to exclude contamination with Varroa-associated viruses through foraged food. In order to minimise possible effects on bee gene expression due through differences in nutrition, both the control Varroa-free and the Varroa-infested colonies were maintained in flight cages in the same apiary (at the University of Warwick, UK) and were fed on the artificial diet for two months before the start of the frame transfer experiments.

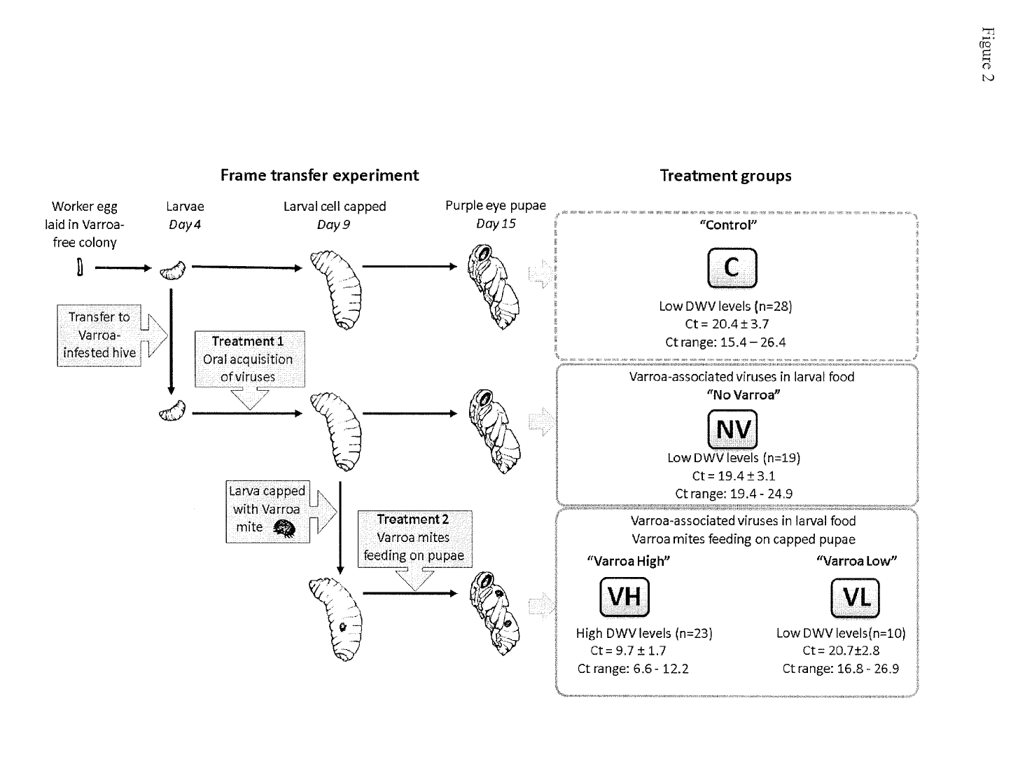

The experimental infestation, summarised in FIG. 2, was conducted on 4th-15th August 2011, and involved the transfer of a brood frame with newly hatched worker bee larvae (on day 4 of development) from the Varroa-free to the Varroa-infested colony. As a result, all the transferred larvae were exposed to the Varroa-selected DWV-like viruses in food delivered by the house bees of the Varroa-infested colony for five days before their capping on day nine of development (FIG. 2 left-hand panel, Treatment 1, Oral DWV infection). Bee larvae continue to develop in the capped cells for six days until sampling at the pupal purple-eye stage on days 15. A proportion of the pupae were capped together with a Varroa mite and were subjected to mite feeding which resulted in direct injection of DWV to bee haemolymph and possible suppression of antiviral responses by Varroa (FIG. 2 left-hand panel, Treatment 2, Mite feeding). The Varroa-infested pupae and the mite groups associated with individual pupae were sampled, with mite feeding on a bee confirmed by the presence of at least one protonymph. Control pupae at the same developmental stage were sampled from the Varroa-free hive at the same time. The pupae and the mites associated with individual pupae were snap frozen in liquid nitrogen immediately after being removed from brood cells and stored at −80° C. prior to total RNA extraction.

In this study we carried out comprehensive analysis of virus diversity and gene expression in the same individual bees which were subjected to oral infection with the strains of DWV associated with Varroa destructor infestation, or both oral DWV in, as well as analysis siRNA and miRNA.

### Real-Time RT PCR

The whole individual honeybee pupae were ground to fine powder in liquid nitrogen, and a half of ground bee was used for RNA extraction was carried out using 1 mL of Trizol Reagent (Invitrogen) according to the manufacturer's instructions. Total RNA extraction from mites was carried out using RNeasy spin columns (Qiagen RNeasy Plant Mini kit). Real-time reverse transcription PCR was carried out essentially as in Moore et al 2011, in brief, RNA extracts were treated with DNAse, then purified DNA-free total RNA preparations were used to as a template to produce cDNA using random primer and Superscript III reverse transcriptase (Invitrogen). The cDNA samples produced were used for real-time PCR quantification of the DWV or host transcripts using SYBR green mix (Agilent Technologies).

Individual bee samples were used to take into account differences in pathogen load and diversity between individuals, as well as to have an opportunity to consider effect of genetic differences on transcriptional responses to the treatments. Bees were analysed bees at the same developmental stage, narrowing to one day, to reduce developmental differences in gene expression which would allow to detect changes induced by parasite and/or pathogens.

Amplification of the cDNA fragments corresponding to the central region of DWV genomic RNA was carried out by nested PCR using GoTaq PCR mix (Promega) and appropriate primers in the cDNA to total RNA samples produced from bee or Varroa mite samples in the pooled bee sample from the Varroa-infested colony used in the transfer experiment.

The outside PCR primers were designed to amplify known DWV strains. For each first round reaction four second round amplification reactions were carried out using VDV-1 or DWV specific primers which allowed VDV-1-type and DWV-type CP and NS to be distinguished, thus amplifying all potential combinations even those which were present at very low levels. The PCR fragments were cloned into plasmid vector, pGemT-Easy (Promega) and sequenced using Sanger dideoxy method.

### Results

Worker honeybees from a Varroa-free colony (sourced in a region with no historic contacts with Varroa), were infected orally with the Varroa-associated DWV strains at the larval stage (from day four until capping on day nine). A proportion of the pupae capped with Varroa mites were also subjected to the mites feeding on their haemolymph during the pupal stage until sampling on day fifteen, five days after capping. Feeding of the mites (adult females) on pupae was confirmed by the presence of at least one protonymph. Sampling capped bee pupae allowed not only identification of the individual bees on which Varroa mites were fed, but also provided bees at the developmental stage when DWV and Varroa mite introduction results in highly pronounced symptoms, such as deformed wings.

The total levels of DWV viruses in each collected pupae was assessed by qRT-PCR using a pair of primers for the conserved polymerase-coding region, designed to detect all known DWV strains, including DWV, VDV-1 and KV. The real-time PCR Ct values showed bimodal distribution, with distinct low DWV level and high DWV level groups (P<0.0001) (FIG. 2 right-hand panel). Low DWV levels were observed in all 23 pupae which were raised in the Varroa-free colony (group C “Control”), in all 19 sampled pupae which were transferred to the Varroa-infested colony but were not capped with a Varroa mite and subjected only to Treatment 1, namely oral DWV infection (treatment group NV, “No Varroa”), and in 10 out of 33 pupae on which Varroa mites were fed. High levels of DWV-like viruses, were detected exclusively in the remaining 23 of 33 Varroa-associated pupae which were subjected to both Treatment 1 and Treatment 2 (group VH, “Varroa High”). The Ct ranges for VH lied entirely below the VL range, indicating the higher virus levels in VH. A similar proportion of the Varroa-associated pupae with low and high levels of DWV were reported previously. The difference in DWV levels between VLand VH could not be explained by different mite loads, there were no difference between the number of adults Varroa female mites in the groups VL and VH (with average 1.375 adult females per cell in each group). Therefore, in order to investigate further what prevented the development of high levels of DWV in these Varroa-infested pupae, we included them in the analysis separately (group VL, “Varroa Low”).

A sample of eight randomly selected bee pupae was taken from each of the four treatment groups for further in-depth analysis of DWV diversity (which included real time qPCR quantification of DWV strains, sequencing of the cDNA clones and next generation sequencing of genomic RNA), transcriptional responses (which included microarray transcriptional profiling and high-throughput micro RNA sequencing), and antiviral RNAi responses (which included high throughput siRNA sequencing).

### Example 2

### Strain-Specific Real-Time PCR Revealed Changes in DWV Populations Following the Oral Infection and Varroa Mite Feeding

The levels of DWV, VDV-1 and their recombinants were quantified in individual pupae and the mites associated with the pupae. Sets of primers designed to distinguish between the DWV-type and VDV-1-type sequences coding for the capsid proteins (CP) and the non-structural proteins (NS) were used (Table 1 below).

The level of the DWV-type and VDV-1-type CP and NS was determined in each of the 32 bees (eight bees in each of the four treatment groups) and in the 15 Varroa mite samples associated with the VH and VL pupae. The 32 bees were also used for the whole genome expression microarray analysis.

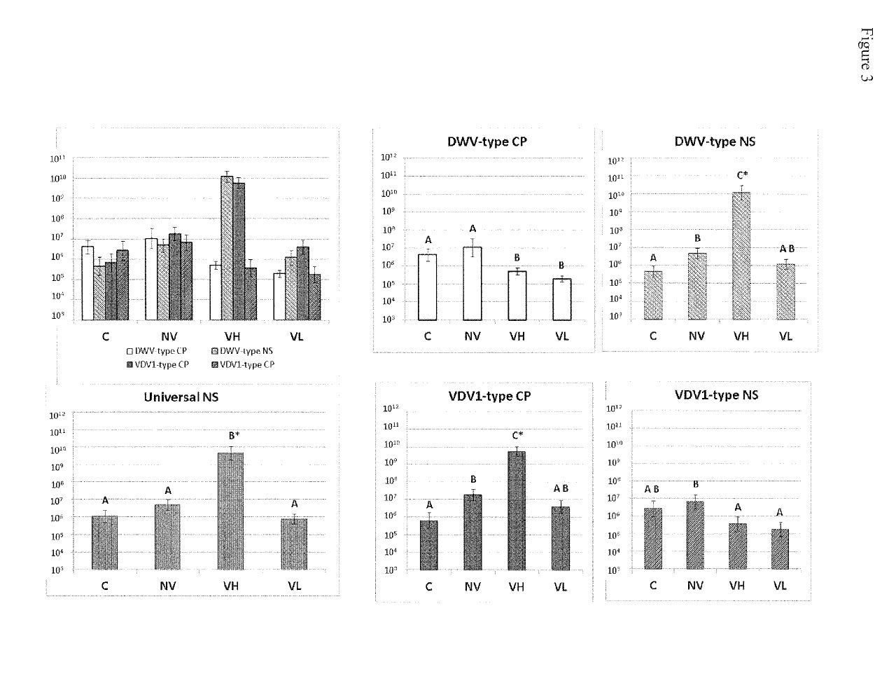

The strain-specific quantification showed that the bees in each of the four treatment groups had unique and significantly distinct combinations of the levels of each of four tested target RNA sequences. The most pronounced was an increase of the number of DWV-like genomes with the VDV-1 CP and the DWV NS sequences in the group VH pupae compared to those in other treatment groups. In comparison with the control group C, the group VH had about a 6,000-fold increase in the VDV-1 CP and about a 26,000-fold increase in the DWV NS. When compared with the group NV, the group VH showed lower relative increases (312-fold for VDV-1 CP and 2500-fold for DWV NS, both with P<0.0001) because an increase of VDV-1 CP and DWV NS also took place in group NV compared to the group C (FIG. 3). The dramatic rise of the recombinant genome(s) containing VDV-1 CP and DWV NS in group VH was accompanied by a statistically significant 30-fold decrease (P=0.0151) of the DWV-type CP and 26-fold decrease (P=0.0477) of the VDV-1 NS when compared with the group NV. The levels of VDV-1 CP and DWV NS showed strong positive correlation (r=0.9691) suggesting preferable replication of VDV-1 CP/DWV NS recombinant(s).

TABLE 1
showing SEQ ID NOs: 11 to 51 respectively

| Sequence (5′-3) | Description |
|---|---|
| GTTTGTATGAGGTTATACTTCAAGGAG | DWV/VDV-1 (8004-8030), F |
| GCCATGCAATCCTTCAGTACCAGC | DWV/VDV-1 (8143-8120), R |
| CTGTAGTTAAGCGGTTATTAGAA | VDV-1 CP (4890-4912), F |
| GGTGCTTCTGGAACAGCGGAA | VDV-1 CP (4986-4966), R |
| CTGTAGTCAAGCGGTTACTTGAG | DWV CP (4917-4939), F |
| GGAGCTTCTGGAACGGCAGGT | DWV CP (5013-4993), R |
| TTCATTAAAACCGCCAGGCTCT | VDV-1 NS (8623-8644), F |
| CAAGTTCAGGTCTCATCCCTCT | VDV-1 NS (8723-8702), R |
| TTCATTAAAGCCACCTGGAACA | DWV NS (8650-8671), F |
| CAAGTTCGGGACGCATTCCACG | DWV NS (8750-8729), R |
| TGAAGGTAGTCTCATGGATAC | Varroa β-actin, R |
| GTCTCTGTTCCAGCCCTCGTTC | Varroa β-actin F |
| CAGTAGCTTGGGCGATTGTTTCG | DWV/VDV-1 (4842-4864), F, |
| CGCGCTTAACACACGCAAATTATC | DWV/VDV-1 (6747-6728), R |
| CTTGGAGCTTGAGGCTCTACA | DWV (6546-6526), R |
| CTGAAGTACTAATCTCTGAG | VDV-1 (6308-6289), R |
| GCCTTCCATAGCGAATTACG | DWV/VDV-1 (9-28), F |
| TTTTCAATTTAATTTTGATTTCGAAGG | DWV/VDV-1 (1092-1118), F |
| CGCCGCCTAGCTTCATCA | DWV/VDV-1 (1245-1228), R |
| GGATGATCCATTTGATAAGG | DWV/VDV-1 (1990-2009), F |
| CATATAGCATCAGAATTAGCCTC | DWV/VDV-1 (2076-2054), R |
| GGGTGCGTAAATATGGTGG | DWV/VDV-1 (3159-3177), F |
| TAGTATCTGAAACAGCTTCC | DWV/VDV-1 (3269-3250), R |
| TGCCTGAGGGCCCTATTGCGAAG | DWV/VDV-1 (4614-4636), F |
| ACCATACCCATATCTTCACGCATC | DWV/VDV-1 (4716-4693), R |
| CAGAGATTGAAGCGCATGAACAAG | DWV/VDV-1 (6474-6497), F |
| GCACTTAACACACGCAAATTATC | DWV/VDV-1 (6750-6728), R |
| GAGTATATACTTATCCATACCATG | DWV/VDV-1 (8028-8051), F |
| CATGCAATCCTTCAGTACCAGC | DWV/VDV-1 (8141-8120), R |
| GAGTAACACCTAACCTGAGTACC | DWV/VDV-1 (10081-10066), R |
| GGGTGTTTACACCGCGATTTATTCG | GB14044, F |
| GATATTTTTTTTCTCCACATTTTTATTCGTCC | GB14044, R |
| CTCTATCTCAAGACCAACACCTACTTGC | GB10640, F |
| CTGATTGTTGGGCACGTCCGGCAACGC | GB10640, R |
| CACAATCTCAAAAATGGATGTTGATACG | GB13140, F |
| AATTAATAACTAATTAGCGAACGAGCAATGG | GB13140, R |
| CTGCAAAATGGAGCCCTCTATGGATTGAG | GB16716, F |
| CAAATAATGCACCTTTGGATCTCTTTTAGCTG | GB16716, R |
| CTTGGTTAGCTGTGTTGCAGTTG | Adapter, F |
| CTTGGTTAGCTGTGTTGCAGTTGCTGTAGTTAAGCGGTTATTAGAA | Adapter-VDV-1 CP (4890-4912), F |
| CTTGGTTAGCTGTGTTGCAGTTGCTGTAGTCAAGCGGTTACTTGAG | Adapter-DWV CP (4917-4939), F |

Primer descriptions are given as follows: target (position in DWV or VDV-1 nucleotide sequence), polarity (F, forward; R, reverse). GenBank accession numbers used to express primer positions are AJ489744 (DWV), NC_{—}006494 (VDV-1), and AB242568 (Varroa destructor β-actin mRNA).

The strain-specific real-time PCR also showed that the oral acquisition of the DWV-like viruses present in a Varroa-infested hive results in changes in virus diversity. The group NV bees had statistically significant 27-fold increase of the VDV-1 CP (P=0.0217) and 10-fold increase of DWV NS (P=0.0314) compared to the Varroa-free control group C.

A significant difference in DWV diversity was observed between the groups NV and VL. Although both these groups contained similar overall levels of the DWV-like viruses (FIG. 2 right-hand panel), there was a statistically significant 54-fold decrease of DWV CP (P=0.0057) and a 36-fold decrease of VDV-1 NS (P=0.0151) in the group VL compared to the group NV. This may suggest that the group VL bees were subjected to a different strain of virus, likely to be delivered by the mite injection.

To determine if increase of the VDV-1 CP/DWV NS recombinants in the Varroa-infested pupae was linked to the increased levels of these recombinants in associated Varroa mites, the strain-specific real-time PCR was carried out in the mite samples. Surprisingly, the viral levels in the mites did not show strong correlation with viral levels in the corresponding bee pupae. The highest statistically significant (P<0.05) correlations were found for the total DWV levels determined using universal NS primers across all samples of the VL and VH groups (r=0.6025) and for the VDV-1 NS levels across the samples of both VH and VL groups (r=0.5342) and for the samples of the VH group (r=0.7850). Notably, no significant correlation was found between the levels of VDV-1 CP and DWV NS in bees and corresponding mites.

### Example 3

### Phylogenetic Analysis of DWV Strains Revealed Reduced Diversity in Overtly Infected Bees and High Virus Variation in the Mites

The real-time PCR quantification of DWV and VDV-1 targets suggested that in the bees of group VH the predominant DWV variants were recombinant genomes with VDV-1 CP and the DWV NS, but this approach did not yield information about the type of these recombinants.

Previously it was found that the recombination points could be located in different positions in the 4500-5500 nt region of the DWV genome. Therefore, sequence analysis of this region was required to provide data on phylogenetic relationships between the DWV strains in the treatment groups, including information on the types of the VDV-1/DWV recombinants. The cDNA fragments corresponding to the central regions of the DWV genome were amplified by nested RT-PCR in the bee or Varroa mite sample pools for each treatment group, as well as in the pooled bee sample from the Varroa-infested colony used in the transfer experiment. The outside primers were designed to amplify known DWV strains including VDV-1 and DWV. For each first round reaction four second round amplification reactions were carried out using the specific primers, which allowed VDV-1-type and DWV-type CP and NS to be distinguished, allowing all potential combinations to be amplified.

Recombinant fragments amplified with the VDV-1 CP and the DWV NS primers were detected in the pupae of all treatment groups as well as in the mite samples, being most abundant in the group VH and the Varroa-infected hive sample. The full-length VDV-1 and DWV sequences were detected in treatment groups C and NV and the Varroa-infested colony sample. Notably, no recombinant sequences with DWV CP and VDV-1 NS parts were detected in any sample. The PCR fragments were cloned into a plasmid vector and 8 to 18 individual clones per treatment group were sequenced. In total 93 individual clones were sequenced, which were aligned with 12 published full-length and nearly full-length sequences of DWV strains including DWV, KV, VDV-1 and DWV-VDV-1 recombinants and used for phylogenetic analysis. The 1330 nt alignment, corresponding to the region 4926-6255 nt of the type DWV strain (GenBank Accession No. AJ489744) had 27% divergent positions (23% of divergent positions among the sequences produced in this study). The high proportion of divergent positions allowed us to generate a robust phylogenetic tree of the central genomic regions of DWV strains.

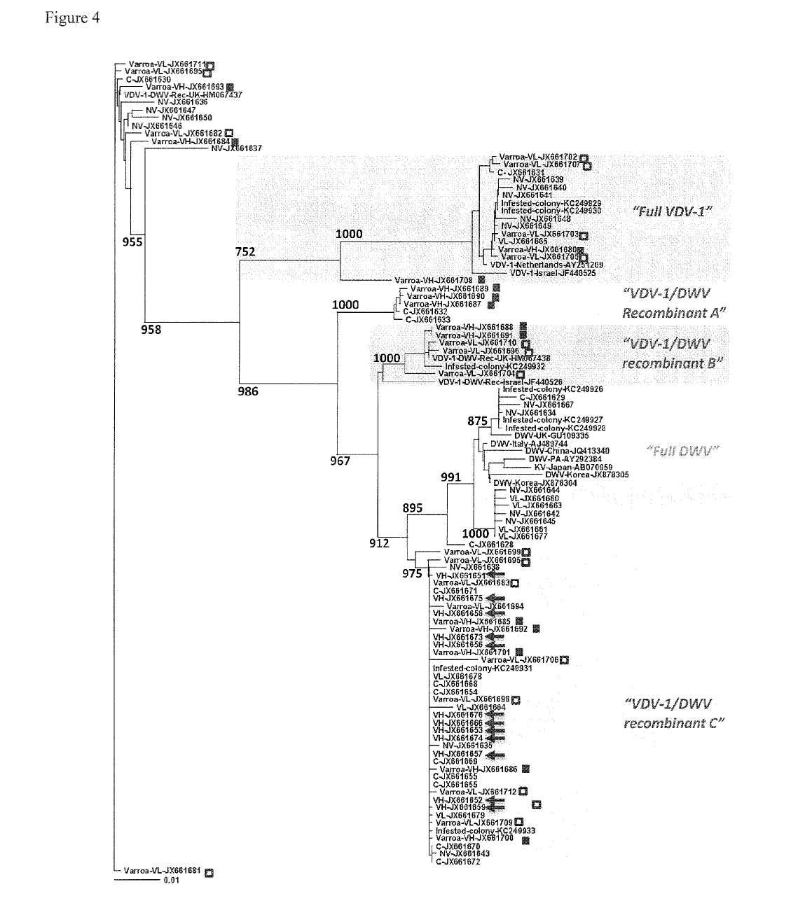

The sequences derived from the bees of the treatment groups C, NV, VL and the Varroa-infested colony appeared in all major clusters of the phylogenetic tree, including DWV, VDV-1 and VDV-1/DWV recombinant clusters, FIG. 4). The presence of the recombinant sequences in different clades may reflect polyphyletic origin of these recombinants, which could be generated with different types of the parental DWV and VDV-1 and/or with the recombination position at different places. All sequences from the group VH bees were confined to a single cluster and were almost identical.

Surprisingly, we found that Varroa mites harboured wider diversity of DWV variants than the pupae on which they were feeding. Although all sequences from the group VH pupae were in a single clade “VDV-1/DWV Recombinant-C” (FIG. 4), sequences from the associated mites were present in all major clades, except the clade “Full DWV”. The sequences from the mites associated with the group VL bees were also present throughout the phylogenetic tree, except “Full DWV” clade (FIG. 4).

### Example 4

### High-Throughput Sequencing of Genomic DWV Genomic RNA

Genomic RNA of DWV and DWV-related viruses was amplified using nested PCR to generate overlapping DNA fragments spanning the entire viral genome. Primers used were directed against conserved regions of the DWV genome and were designed to amplify all known DWV-like viruses, including DWV, VDV-1, KV. Uncloned cDNA populations were sequenced commercially (GATC Biotech.) using standard library preparation methodologies. Paired end sequence data (˜5 million reads per cDNA fragment) was assembled using tview and Bowtie by alignment with reference sequences from Genbank (DWV reference NC_{—}004830) and previous recombinant cDNAs sequenced by the inventors (Moore et al. 2011 J. Gen. Virol. 92: 156-161, Genbank HM063437 and HM063438).

Sequence reads were used to generate a consensus sequence for the majority DWV-like virus present in the preparation. Two predominant forms exist, differing only in their 5′ non-coding regions. The predominant forms were both recombinants in which the structural proteins were more closely related to VDV and the non-structural proteins to DWV. The two recombinant forms had either DVD-like or VDV-like 5′ non coding regions. The sequences for the two predominant forms are given in SEQ ID NOs: 1 and 2.

Sequence variation was determined by analysing all aligned reads showing mismatches to the consensus and templates sequences. ˜81% of all reads in the library were DWV-like. The overall variation of the predominant virus sequence was determined as 0.99051%.

### Example 5

### High-Throughput Sequencing of DWV siRNA Populations Suggested Differential Targeting of DWV and VDV-1-Derived RNA Sequences

### Results

The small RNA (15 to 40 nt) fractions were isolated from the bee total RNA samples (which were used for real-time PCR), pooled according to the treatment groups and used for small RNA library preparations and small RNA sequencing. Libraries which contained 15 to 25 million reads were aligned to the reference DWV and VDV-1 sequences using Bowtie (Langmead B et al. Genome Biol 10:R25.).

DWV and VDV-1 specific siRNAs of both polarities were present in all treatment groups. The most abundant siRNA species were 21 and 22 nt in length, consistent with previous reports on siRNA in insect and suggesting that Dicer operated bees in all treatment groups. It is generally believed that in insects siRNAs are produced by processing of dsRNA replication intermediates and no amplification of siRNA takes place due to the lack of host-encoded RNA-dependent polymerase gene(s). The presence of the DWV-specific 21-22 nt siRNA derived from both positive and negative strands was an indication that replication of these viruses takes place in developing bees of all groups, including those with the low levels of DWV-like viruses (“Control” C, and “No Varroa” NV).

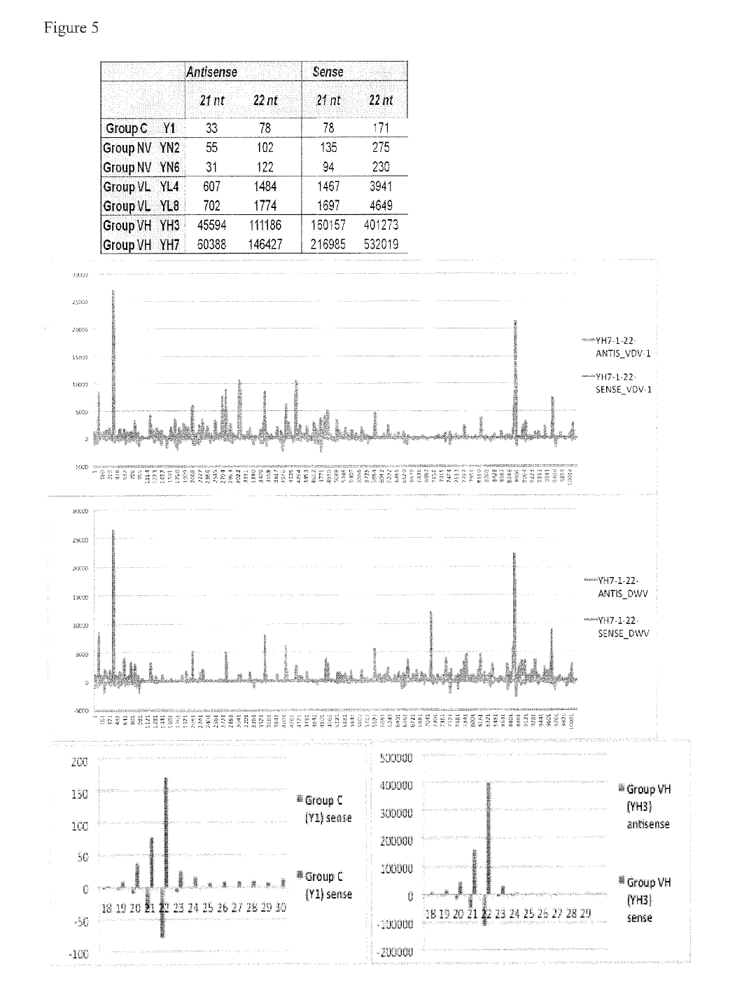

Most of the reads were 21 or 22 nt long, with the three to four times more reads being produced from positive RNA strand than from negative RNA strand (FIG. 5 and Table 2 below). The numbers of 21 nt and 22 nt DWV and VDV-1-specific reads varied from about 350 in the control group C to about 900,000 in the high DWV Varroa-infested group VH1.

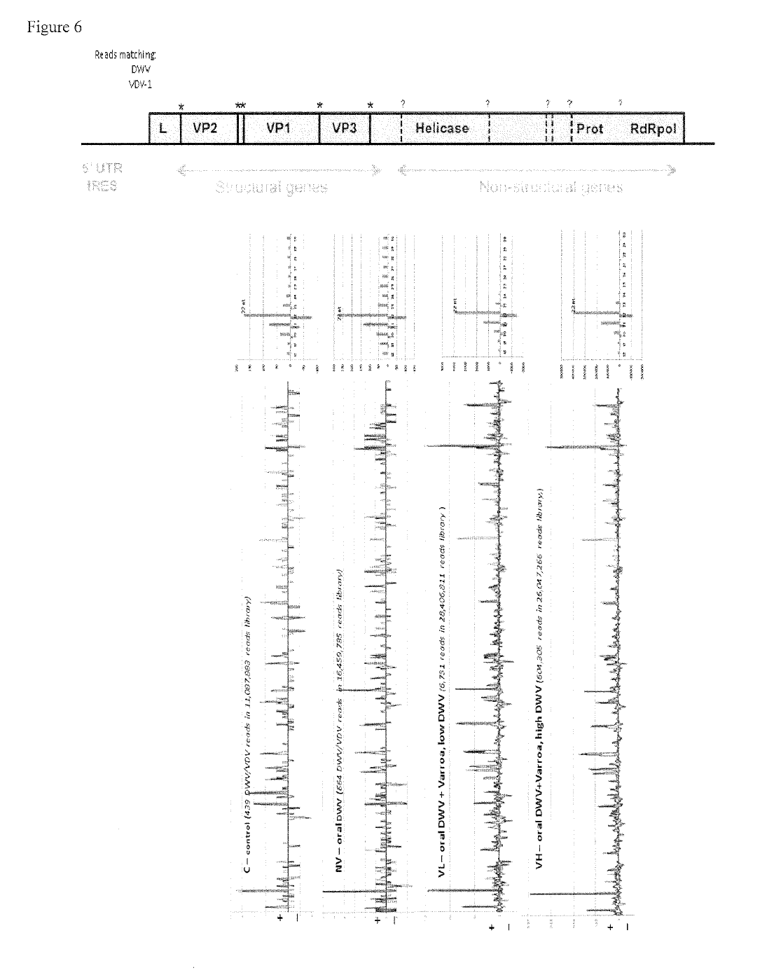

Pools of siRNA were selected which were unambiguously matching either DWV or VDV-1 reference sequences and profiles of the DWV-type and VDV-1-type siRNA for the libraries derived from the pooled samples of the groups C, NV, VH, and VL were created and analysed (FIG. 6). RNAi is likely to be a major defence mechanism operating in insects. RNA viruses encode suppressor of RNAi. The presence of DWV and VDV-1-specific siRNAs in bee pupae suggest that RNAi may operate against DWV-like viruses. It cannot be excluded, however, that DWV encodes suppressors of RNAi which operate downstream of the production of siRNA and, possibly, incorporate into the Argonaute complex. For example, suppression may target action of the RISC complex.

TABLE 2
Summary of the small RNA sequencing in FIG. 5

| Treatment group | C | NV | NV | VL | VL | VH | VH |
|---|---|---|---|---|---|---|---|
| Small RNA library | Grop C-1 | Group NV-2 | Group NV-2 | Group VL-1 | Groip VL-2 | Groip VH-1 | Group VH-2 |
| Total reads | 11087883 | 16459758 | 13102754 | 28406811 | 32232127 | 26047266 | 35064538 |
| miRNA reads | 1585149 | 2655959 | 2084357 | 5110815 | 5572315 | 3160162 | 4179594 |
| miRNA reads % | 14.3% | 16.1% | 15.9% | 18.0% | 17.3% | 12.1% | 11.9% |
| Total number of DWV | 540 | 1001 | 828 | 9844 | 11513 | 900332 | 1198197 |
| and VDV-1 reads: | | | | | | | |
| DWV, VDV-1 sense reads | 407 | 806 | 615 | 7214 | 8385 | 710809 | 947468 |
| DWV, VDV-1 antisense reads | 133 | 195 | 213 | 2630 | 3128 | 189523 | 250729 |
| DWV and VDV-1 reads per | 0.341 | 0.377 | 0.397 | 1.926 | 2.066 | 284.901 | 286.678 |
| 1000 miRNA reads | | | | | | | |
| | 25% | 19% | 26% | 27% | 27% | 21% | 21% |
| | 75% | 81% | 74% | 73% | 73% | 79% | 79% |
| DWV coverage | 9434 | 12934 | 10327 | 114715 | 138700 | 9906028 | 13212790 |
| VDV-1 coverage | 6715 | 11507 | 9123 | 123321 | 142988 | 10850253 | 14609501 |
| Coverage ratio, | 1.405 | 1.124 | 1.132 | 0.930 | 0.970 | 0.913 | 0.904 |
| DWV/VDV-1 | | | | | | | |
| Number of DWV and | | | | | | | |
| VDV-1 small RNAs | | | | | | | |
| Total sense (%) | 407 (75%) | 806 (81%) | 615 (74%) | 7214 (73%) | 8385 (73%) | 710809 (79%) | 947468 (79%) |
| 18 nt sense | 12 | 30 | 32 | 141 | 134 | 10553 | 14064 |
| 19 nt sense | 10 | 42 | 29 | 200 | 220 | 17273 | 22713 |
| 20 nt sense | 38 | 62 | 47 | 488 | 561 | 46599 | 61970 |
| 21 nt sense | 78 | 135 | 94 | 1467 | 1697 | 160157 | 216985 |
| 22 nt sense | 171 | 275 | 230 | 3941 | 4649 | 401273 | 532019 |
| 23 nt sense | 26 | 55 | 50 | 416 | 501 | 32333 | 43047 |
| 24 nt sense | 17 | 28 | 21 | 106 | 132 | 7292 | 9616 |
| 25 nt sense | 7 | 43 | 24 | 99 | 97 | 5656 | 7592 |
| 26 nt sense | 9 | 29 | 19 | 85 | 76 | 5804 | 7938 |
| 27 nt sense | 9 | 25 | 15 | 81 | 85 | 5933 | 7952 |
| 28 nt sense | 10 | 29 | 23 | 68 | 85 | 5653 | 7350 |
| 29 nt sense | 7 | 27 | 14 | 61 | 56 | 5839 | 7659 |
| 30 nt sense | 13 | 26 | 17 | 61 | 92 | 6444 | 8563 |
| Total antisense (%) | 133 (25%) | 195 (19%) | 213 (26%) | 2630 (27%) | 3128 (27%) | 189523 (21%) | 250729 (21%) |
| 18 nt antisense | 2 | 2 | 3 | 42 | 47 | 2184 | 3049 |
| 19 nt antisense | 4 | 9 | 5 | 80 | 127 | 5705 | 7818 |
| 20 nt antisense | 9 | 15 | 31 | 246 | 302 | 16295 | 21826 |
| 21 nt antisense | 33 | 55 | 31 | 607 | 702 | 45594 | 60388 |
| 22 nt antisense | 78 | 102 | 122 | 1484 | 1774 | 111186 | 146427 |
| 23 nt antisense | 7 | 3 | 12 | 155 | 145 | 7581 | 9989 |
| 24 nt antisense | 0 | 3 | 4 | 6 | 19 | 553 | 707 |
| 25 nt antisense | 0 | 1 | 0 | 2 | 9 | 131 | 174 |
| 26 nt antisense | 0 | 0 | 1 | 3 | 1 | 64 | 90 |
| 27 nt antisense | 0 | 1 | 1 | 1 | 0 | 46 | 56 |
| 28 nt antisense | 0 | 2 | 2 | 2 | 1 | 51 | 70 |
| 29 nt antisense | 0 | 0 | 1 | 1 | 0 | 54 | 64 |
| 30 nt antisense | 0 | 2 | 0 | 1 | 1 | 79 | 71 |

### Example 6

### Generation of Transgenic Bees

### Outline

Honeybees (Apis mellifera) can be engineered to express foreign sequences, stably integrated into their genome, by retroviral vector-mediated infection of spermatozoa and instrumental insemination. Transgenic honeybees, resistant to acute infection with pathogenic viruses such as deformed wing virus (DWV), may be created that express regions of the virus genome engineered in such a way that they induce small interfering RNA (siRNA or RNAi) molecules. For this resistance to be inheritable the virus sequences will need to be engineered into the genome of the honeybee.

### Background

To introduce novel genetic material into the germline of honeybees it will be necessary to manipulate a queen to lay fertilized eggs containing the novel genetic material. Sperm mediated gene transfer (SMGT) in honeybees has been achieved in the art by mixing plasmid DNA with spermatozoa used for instrumental insemination. The sequences introduced were stably maintained for several months but did not integrate into the genome. The introduced sequences were also passed on to progeny raised from the manipulated queen. It appears as though a limitation to this approach is the absence of genome integration.

In the present application, to circumvent this limitation, retroviral mediated infection of honeybee spermatozoa is used. This approach has been used to generate transgenic animals from other species, such as zebrafish. Retroviruses are RNA viruses that integrate into DNA during their replication cycle. Virologists have developed methods to create novel retroviruses carrying different ‘payloads’ e.g. foreign genes. To ensure expression of these foreign genes a suitable promoter must be present.

### Method

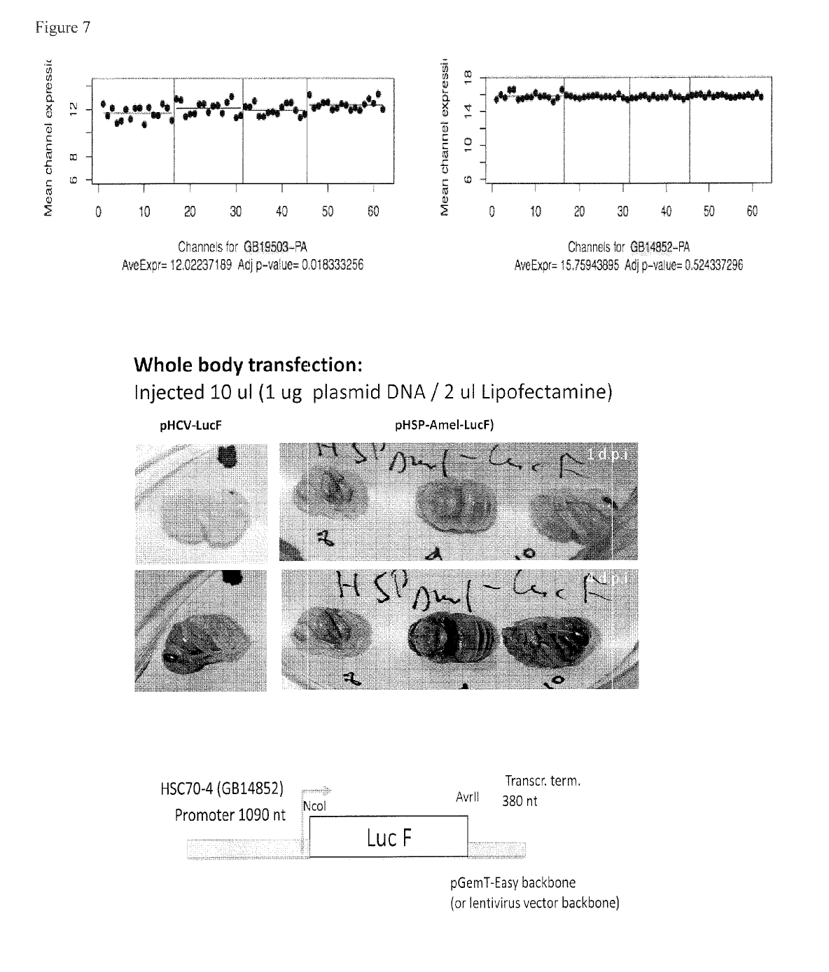

A functional honeybee promoter from a heat shock protein (hsp70) has been identified and cloned (SEQ ID NO: 8). This promoter has been demonstrated to function upon microinjection of bumblebee (Bombus) pupae. A recombinant retrovirus genome (designated plasmid 1) has been generated based upon a standard lentivirus (such as human immunodeficiency virus (HIV), several of which are available commercially) which contains the hsp70 promoter adjacent to a luciferase reporter gene. The sequence of this recombinant retrovirus genome is given in SEQ ID NO: 10. Whole body transfection of bee larvae with the recombinant retrovirus has been shown to result in luciferase expression within the bee larvae (FIG. 7).

To generate recombinant retrovirus particles suitable cells e.g. mammalian HeLa cells, are co-tranfected with plasmid 1 together with a plasmid (designated plasmid 2) encoding a suitable envelope glycoprotein (for example, the envelope glycoprotein from vesicular stomatitis virus (VSV) which (a) is usually used to create such recombinant retrovirus particles and (b) is known to attach to and allow infection of insect cells) and a plasmid (plasmid 3) encoding the gag and pol genes of a suitable retrovirus. With the exception of the newly-identified honeybee promoter, which is disclosed herein for the first time, this technology is all known in the art and available commercially.

The resulting recombinant retrovirus is mixed with donor spermatozoa harvested from suitable drones and used for retroviral vector-mediated infection of spermatozoa and instrumental insemination of virgin honeybee queens. The resulting fertilized queens are used to establish small colonies and the progeny screened for the presence of introduced foreign DNA (e.g. luciferase).

### Example 7

### Inoculation of Honeybees by Injection into Haemolymph Results in Preferential Amplification of Particular VDV-1/DWV Recombinants

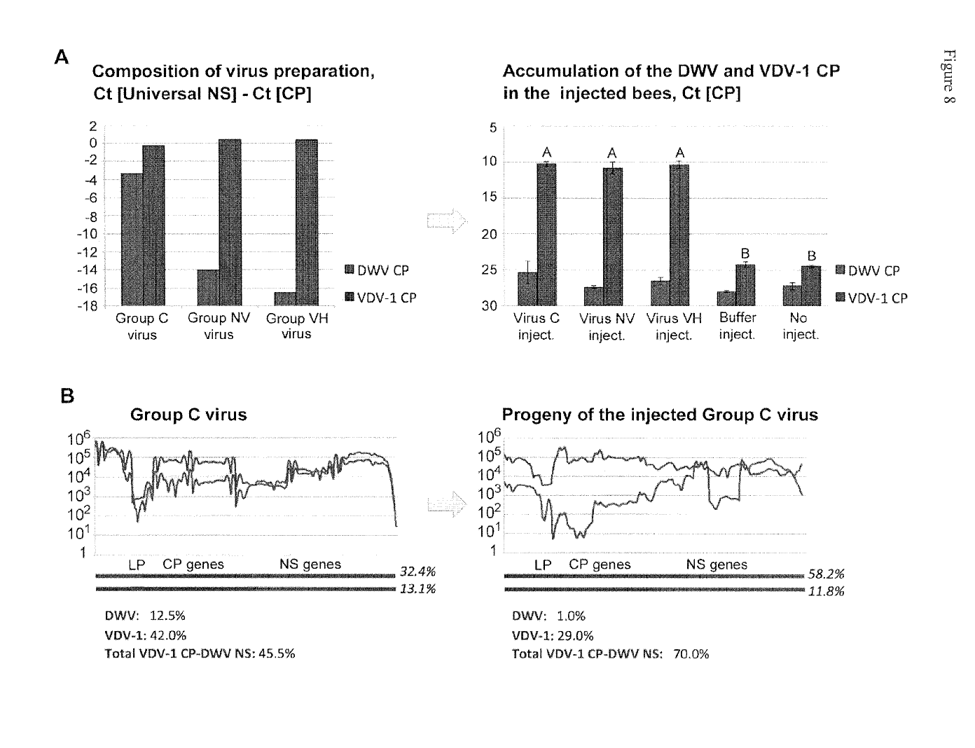

White eye pupae (day 12-13 of development) maintained in vitro (as described in Mockel et al. 2011 J Gen Virol 92: 370-377) were directly injected with virus particles purified from groups C, NV and VH pupae according to Moore et al (2011). The proportion of the DWV- and VDV-1-type CP coding regions in the inocula and injected pupae (following incubation to the blue eye stage for 3 days) were determined by qRT-PCR using strain-specific primers to the CP and universal primers to the NS region. All preparations contained higher and broadly similar levels of VDV-1-like CP coding regions. In contrast, the amount of DWV-like CP coding regions was much higher in the virus preparation from the group C pupae (where it accounted for ˜12% of the population) than from either the NV or VH group pupae (FIG. 8A). Pupae inoculated with buffer alone exhibited no significant increased accumulation of DWV-like viruses when compared with untreated pupae. In contrast, irrespective of the viral inocula, all recipient pupae injected with virus preparations exhibited high virus levels (as determined by Ct values) similar to those previously observed in the VH experimental group (FIG. 3 top left panel and FIG. 2).

Next generation sequencing (Illumina paired end reads) was conducted to more completely characterise the group C inocula and the viruses present in pupae injected with the group C virus. The composition of the inoculum, as determined by qRT-PCR and subsequent MosaicSolver analysis of the NGS reads, were in close agreement and consisted of 12.5% DWV, 42% VDV-1 with the remainder being a VDV-1 CP-encoding recombinants with a DWV-like NS region (FIG. 8B). In addition, these recombinants were both carried 5′ sequences that were DWV-like (approximately two thirds of the RFs contained a DWV-like 5′ NCR and Leader protein [LP] coding region with the remainder carrying a VDV-1-like 5′NCR and DWV-like LP).

Pupae inoculated with group C virus exhibited a marked reduction in the DWV content (1%) and a concomitant statistically significant increase in recombinant forms of the virus (70% of the total) that were characterised by the presence of VDV-1 CP coding region and DWV-like NS regions (FIG. 8B). Again, two recombinant forms predominated, though these were different from those in the inocula and could be distinguished by possession of an entire 5′ NCR and LP region from VDV-1 and two distinct crossovers in the helicase-coding domain of NS region (FIG. 8B). These results further support the conclusion that DWV-like viruses bearing VDV-1 CP coding regions, and particularly recombinants forms with DWV-derived NS coding regions, are advantaged in a Varroa-infested honeybee colony, but additionally indicate that this advantage is manifest after transmission of the virus by direct inoculation and is not dependent upon Varroa per se.

### Example 8

### A Virulent Strain of Deformed Wing Virus (DWV) of Honeybees (Apis mellifera) Prevails after Varroa destructor-Mediated, or In Vitro, Transmission

Host-pathogen interactions can be broadly divided into asymptomatic or symptomatic infections [1]. In the former, the absence of symptomatic disease is typically due to restricted pathogen replication, which reduces the opportunities for horizontal transmission within its host population. Conversely, prolonged survival of the infected host increases the likelihood of vertical transmission of the pathogen[2]. In contrast, symptomatic infections are typically characterized by high levels of pathogen replication, with consequent enhanced virulence, thereby maximizing horizontal transmission [1-4]. The ‘lifestyle choice’ of asymptomatic or symptomatic infection is determined by multiple factors including the duration of host-pathogen co-evolution, host physiology and anti-pathogen responses, routes of transmission and environmental factors. Evolutionary changes in pathogen virulence may be triggered by changes in pathogen-host assemblages [5]. In the case of multi-host pathogens with interspecies transmission, a pathogen's virulence may increase following introduction of a second host, when the constraint on pathogen virulence in a given host is removed [6].

The European honeybee (Apis mellifera) is the predominant managed pollinating insect and delivers economically important pollination services for agriculture which are estimated to add ˜$40 bn globally to crop value/annum [7]. Factors that influence colony health and viability are therefore important for colony survival and pollination performance. In addition to the bacterial foulbroods, the most important diseases of A. mellifera are caused by a range of viruses many of which are vectored by the ectoparasitic mite Varroa destructor when feeding on honeybee haemolymph. Varroa is believed to have expanded its host range from Apis cerana to A. mellifera during the first half of the 20^{th} century and subsequently spread to all beekeeping regions of the world with the exception of Australia [8-11].

Deformed wing virus (DWV), a picorna-like single-stranded, positive-sense, RNA virus [12,13], is present in the majority of honeybee colonies [10]. DWV is closely related to Varroa destructor virus type 1 (VDV-1) [14]. Their recombinants [15,16] and Kakugo virus (KV) [17], which together exhibit at least 84% nucleotide identity, can be considered as strains of the same virus (henceforth we use the term DWV to refer to this related group of viruses). In the absence of Varroa, DWV generally causes asymptomatic infection and is present at low levels in honeybees. In contrast, in Varroa-infested colonies, mite-exposed pupae can exhibit very high DWV levels which may result in impaired development of the general adult honeybee and increased mortality [10,13]. The mechanisms underlying the transition of DWV from a relatively benign virus to a major honeybee pathogen in the presence of Varroa remain unclear. Two possibilities, not mutually exclusive, have been proposed: suppression of honeybee antivirus defences by Varroa mites which allows the virus to proliferate [18,19], and a Varroa-driven selection of particular DWV genotypes, potentially due to replication in the mite [15,20].

Previous studies using functional or gene expression analysis have produced contradictory conclusions on the impact of Varroa on the immune responses of honeybees. Initial reports indicated that Varroa-exposed honeybees were immuno-compromised [18,19], although later transcriptome analysis found little or no effect on genes implicated in insect immunity [21,22]. Additional studies have shown down-regulation of a honeybee NF-κB transcription factor [23]. Recent reports have implicated the Drosophila Toll, Imd and Jak-Stat signalling pathways in controlling RNA virus infection [24] and RNA interference (RNAi), which has long been considered the major antiviral mechanism in insects [25], has recently been associated with controlling the persistence of RNA virus infections in Drosophila [26]. It was therefore possible that high levels of DWV in Varroa-exposed honeybees could be the result of a suppression of these antivirus responses and so warranted further analysis.

We have previously demonstrated that Varroa infestation is associated with the accumulation in mite-exposed pupae of a particular subset of DWV-like viruses [15]. These recombinant forms (RF) are predominantly comprised of genomes with structural and non-structural coding regions that most closely align with VDV-1 and DWV respectively. The organisation of these recombinants suggests that, as with other picorna-like viruses, DWV likely has a modular genome, with a 5′ untranslated region (5′-UTR) driving translation of the structural or capsid (CP) and non-structural (NS) ‘modules’ [15]. We hypothesised that such recombinants were transmitted more efficiently between Varroa and honeybees, resulting in their amplification to the markedly elevated levels observed in Varroa-parasitized pupae (about 1000 times higher than in unexposed pupae). In recent complementary studies, changes in the composition of the DWV population over a large temporal and spatial scale following Varroa infestation were reported for honeybees colonies following accidental introduction of Varroa into the Hawaiian islands [20]. The introduction of Varroa was associated with a marked restriction in DWV diversity measured in the pooled honeybee samples collected from the Varroa-infested colonies, although the precise identity of the dominant virus was not determined [20].

In the present study we devised a novel experimental system to specifically test two hypotheses on the role of Varroa in the development of high-level DWV infection in the honeybee, namely that the mite (i) amplifies and transmits virulent genotypes of DWV, and (ii) suppresses antiviral responses, including immune signalling pathways and RNA interference. The experimental procedure included exposure of Varroa-naïve honeybees to mites and their associated DWV payload together with the per os in-hive horizontal transmission. The use of Varroa-naïve honeybees from a Varroa-free region allowed us to monitor changes in DWV diversity and loads, as well as potential antivirus responses in the honeybee responses, following exposure to the viral genotypes associated with Varroa infestation. Importantly, we analysed immune responses and viral load/diversity in individual mite-exposed and -unexposed pupae, rather than in pooled samples. This allowed us to stratify individual responses into four distinct experimental groups, characterised by Varroa exposure and viral load, that clearly correlated with characteristic changes in the transcriptome and virus population diversity. In addition, we recapitulated the exposure of Varroa-free honeybees to DWV by direct injection and analysed virus diversity in bees of a colony with long-established Varroa infestation.

Our results indicate that a virulent recombinant form of DWV, while transmissible orally, only replicates to high levels when directly inoculated into honeybee haemolymph—by Varroa or experimental injection. This results in massive reduction of DWV diversity in bees with high virus levels, both in the Varroa-exposed pupae and newly emerged bees with symptomatic deformed wing disease. Significantly, the same virulent recombinant form of DWV reached the highest levels in mite-exposed pupae and in adult bees exhibiting characteristic deformed wing symptoms. Although exposure to Varroa resulted in changes in expression of a number of immune-related genes, the roles of which should be further explored, we demonstrate that it is the route of virus acquisition that is responsible for the amplification of this virulent form of DWV in a Varroa-infested colony.

### Results

### Experimental Infestation by Varroa Mites Results in Bimodal DWV Levels

Worker honeybee larvae from a Varroa-free colony (sourced from a region with no historic contacts with or presence of Varroa) were moved in a frame transfer experiment to a Varroa-infested colony. The larvae were subsequently exposed through feeding to DWV strains circulating in the infested colony from day 4 until the cells were capped at day 9 (all times relative to egg laying; FIG. 2, Treatment 1). Varroa mites enter brood cells immediately prior to capping. Therefore, pupae located within brood cells that contain Varroa mites are also subjected to the mite feeding on haemolymph during pupal development (FIG. 2, Treatment 2) until sampling on day 15 (the purple-eye stage), six days after cell capping. Feeding of the mites (adult females) on pupae was confirmed by the presence of at least one protonymph in the capped cell [27].

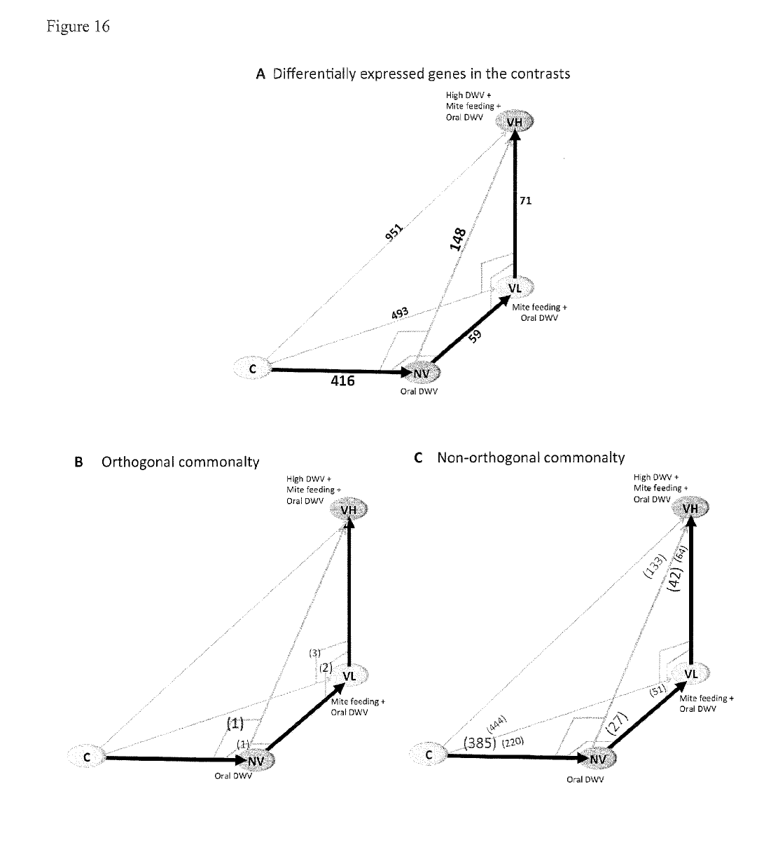

We assessed the total levels of DWV viruses in 80 individual pupae by qRT-PCR using a primer pair for a conserved polymerase-coding region, designed to detect all known DWV strains, including DWV, VDV-1 and KV (Tables 1 and 5). The real-time PCR Ct values showed a clear bimodal distribution, with distinct low- and high-levels of DWV (p<10⁻¹⁶; FIG. 2, FIG. 15). Low DWV levels were observed in all (n=23) sampled pupae maintained in the Varroa-free colony (group C, “Control”), in all 19 sampled pupae transferred to the Varroa-infested colony that were not capped with a Varroa mite and therefore subjected only to Treatment 1 (oral DWV infection; group NV, “No Varroa”), and in 10 of 33 pupae upon which Varroa mites had fed, Treatment 2 (group VL, “Varroa Low”). In contrast, high levels of DWV-like viruses were detected in the remaining 23 of 33 Varroa-associated pupae, which experienced both Treatment 1 and Treatment 2 (group VH, “Varroa High”). The Ct ranges for the VH group lay entirely below the VL range, indicating significantly higher virus levels in VH (FIG. 15A) whereas the Ct values in groups C, NV and VL were statistically indistinguishable (ANOVA). We have previously reported similar proportions of Varroa-associated pupae with low and high levels of DWV in an independent (temporally and geographically) study [15]. These results indicate that direct Varroa exposure does not inevitably lead to high, presumed pathogenic, DWV levels, as reported previously [15,28,29], at least when age-matched, synchronously exposed pupae are analysed individually. The difference in DWV levels between pupae in the VL and VH groups could not be explained by different mite loads—both contained an average of 1.375 adult female Varroa mites per cell (data not shown). These two distinct classes of Varroa-exposed pupae, and their associated mites, were included as separate groups in subsequent analyses to investigate host or parasite determinants that influenced the outcome of exposure.

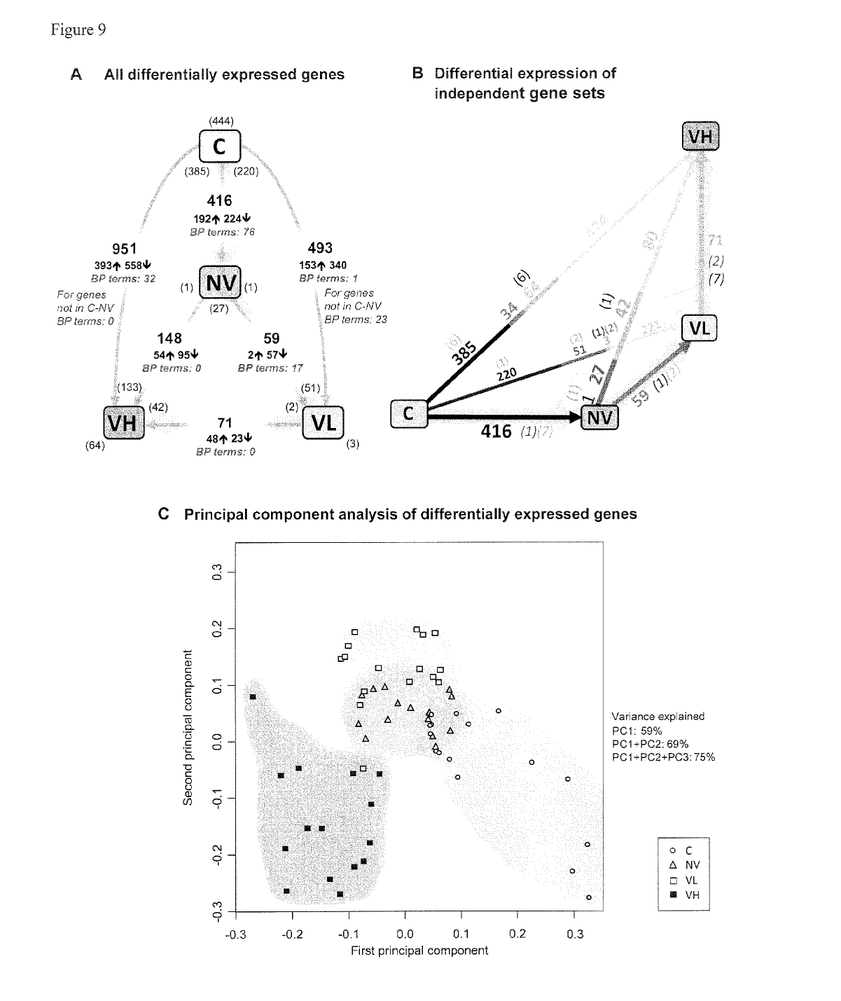

We sampled eight honeybee pupae selected at random from each of the four groups (C, NV, VH and VL; FIG. 9) for further analysis. With the exception of the siRNA responses (for which pooled samples of each of the four groups were used), subsequent analysis of transcriptional responses (microarray transcriptional profiling) and virus diversity (qRT-PCR, cloning and sequencing) were conducted individually on each of the eight pupae from the four response groups.

### Significant Changes to the Honeybee Transcriptome are Characteristic of Experimental Groups and Varroa/Virus Exposure

We used a two-colour dye-balanced loop design microarray [30,31] to determined the genome-wide transcriptional profile using RNA extracted from the 32 samples defined above, (8 pupae from each experimental group). The oligonucleotide expression array contained probes to all protein-coding transcripts of A. mellifera [32], as well as probes to all known viral and fungal pathogens of honeybees, including distinct DWV and VDV-1 probes. After array normalization, differentially expressed (DE) genes were determined for each contrast between experimental groups (FIG. 9A). Microarray results were validated by qRT-PCR using oligonucleotide primers to a set of honeybee DE genes and the constitutively expressed ribosomal protein 49 (Rp49) gene (GB10903; Table 5), showing strong positive correlations between the processed microarray signals and normalized Ct values (Pearson correlation coefficients between 0.504 and 0.873). Additionally, there was a strong positive correlation between the DWV microarray signal and qRT-PCR Ct values for DWV-like viruses using generic DWV primers (Table 1, SEQ ID NOs: 11 and 12), Pearson correlation coefficient 0.797. Other than DWV-like viruses, no other honeybee pathogens were detected.

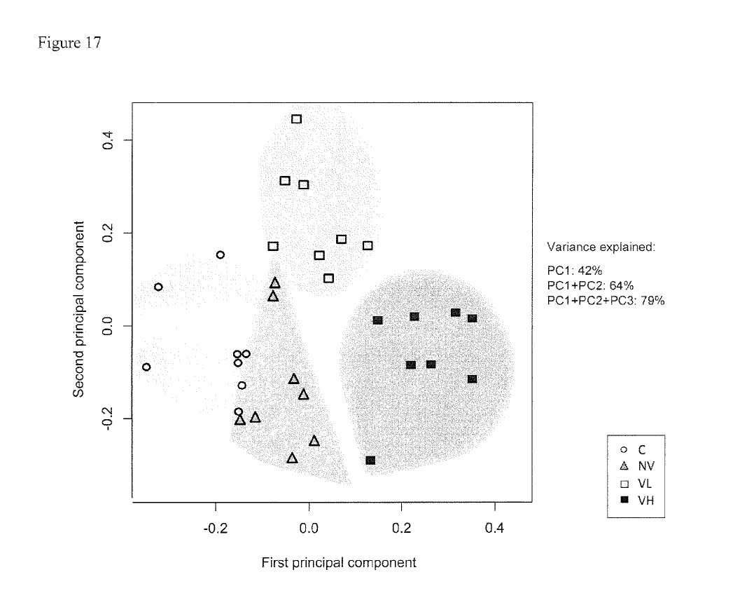

There were high levels of commonality and additivity for DE genes in the contrasts considered (FIG. 9A, FIG. 16 A, C). For example, the C to VH contrast (in which ˜10% of genes were DE) can be decomposed into two sub-contrasts by exposure regime, i.e. split C to VH at oral exposure (NV) or at mite feeding (VL). Similarly the C to VL contrast can be split at NV. These decompositions exhibit high orthogonality (FIG. 9 A,B; FIG. 16 B,C). This suggests that expression of essentially different sets of genes are influenced following oral exposure to DWV, Varroa feeding, and the markedly elevated levels of DWV in Varroa-exposed pupae. To explore this further we conducted principal component analysis (PCA). Distinct clustering by experimental group was observed when two independent sets of DE genes with the lowest p-values were analysed i.e. those from the DE genes pooled from all contrasts (FIG. 9C), or DE genes in each of 6 contrasts (FIG. 17). Consequently, PCA strongly suggests that the experimental groups exhibit characteristic gene expression signatures reflecting their fate after exposure in a Varroa-infested colony.

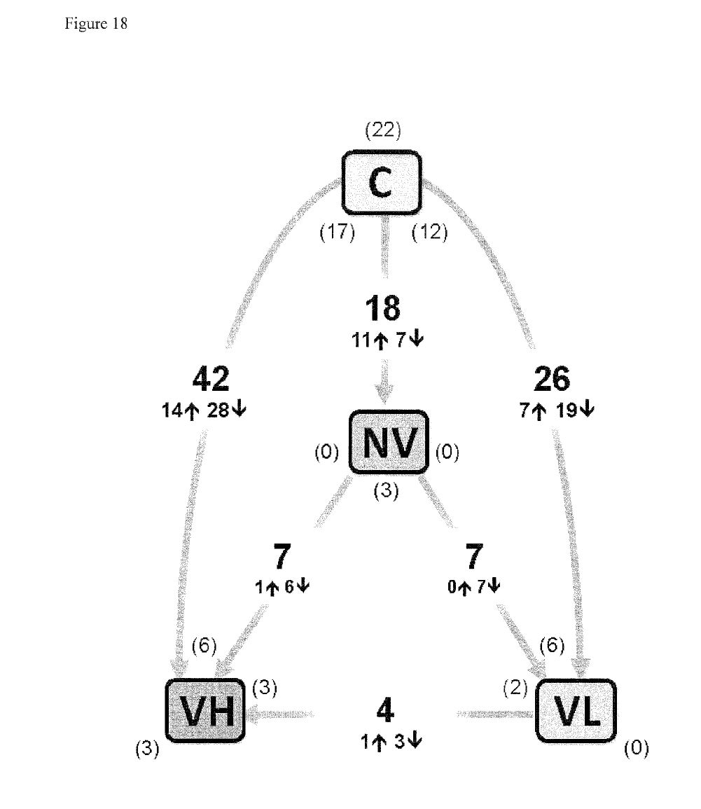

To obtain insight into the functional consequences of DE gene expression we carried out Gene Ontology (GO) analysis, focusing on the GO Biological Process (BP) [33]. A number of overrepresented GO BP terms related to cell division were associated with DE genes in the C to NV contrast, while those related to regulation of various cellular processes were associated with the DE genes in the NV to VL contrast (FIG. 9A). Notably, no overrepresented GO BP terms were associated with the genes DE following increase of DWV levels (VL to VH contrast). We then looked in detail at the expression patterns of likely immune-related genes as it had previously been reported that Varroa and/or viruses could influence honeybee immunity [18,19,23]. The list of 381 putative honeybee immune-related genes included those previously published [34,35] together with honeybee homologs of the Drosophila genes associated with the GO term “Immune system process” (GO: 0002376). The C to VH and C to VL contrasts exhibited the highest number of DE immune-related genes (n=42 and n=26 respectively, 22 of the latter also being in the C to VH contrast), whereas oral exposure (C to NV) resulted in 18 DE immune-related genes (Table 3, FIG. 18). Independent confirmation of DE of immune-related genes was obtained by qRT-PCR analysis of persephone protease (GB 14044), Tollo (GB 10640), and Vago (GB 10896) with Pearson correlation coefficients of 0.598, 0.504 and 0.692 respectively.

### Next Generation Sequencing Analysis of the RNAi Response in DWV-Infected Pupae

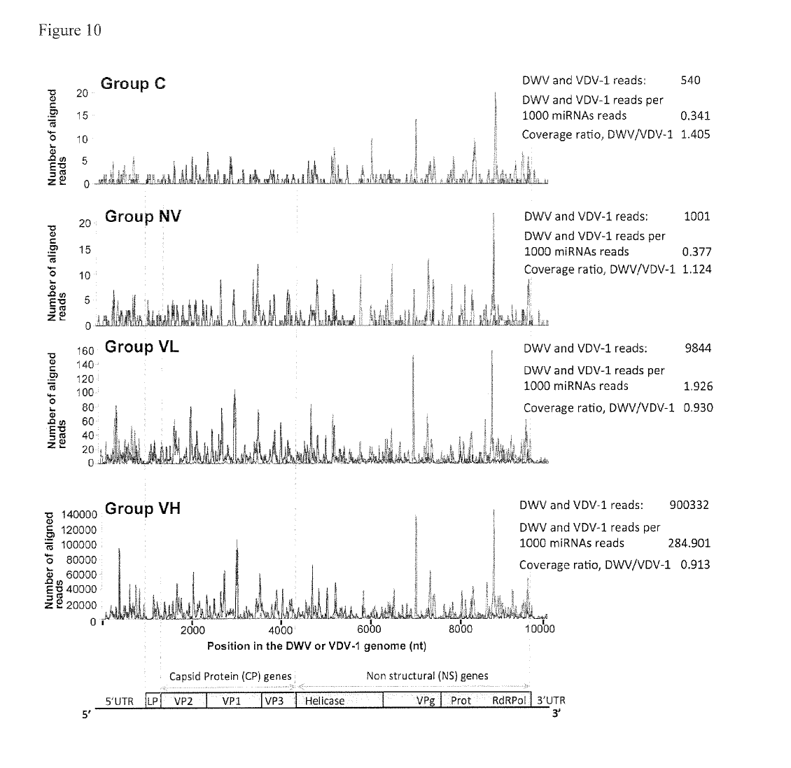

Although no significant changes in expression of genes associated with the RNAi response (e.g. Argonaute, Dicer) [25] were observed in the microarray analysis, there could be post-transcriptional effects on RNAi generation. We therefore analysed the DWV-related RNAi population and compared it with the levels and identity of virus in pupae from the four experimental groups (FIG. 9). Small RNA fractions (15 to 40 nt) were isolated from total RNA samples, pooled according to the experimental groups and used as templates for Illumina high-throughput sequencing. One library was generated for the group C honeybees and two libraries for each of the other groups. These libraries, each containing 11 to 35 million reads, were aligned to the reference viral sequences (DWV and VDV-1, GenBank accession numbers GU109335 and AY251269 respectively; FIG. 10), as well as to the honeybee miRNA sequences [36], using Bowtie [37].

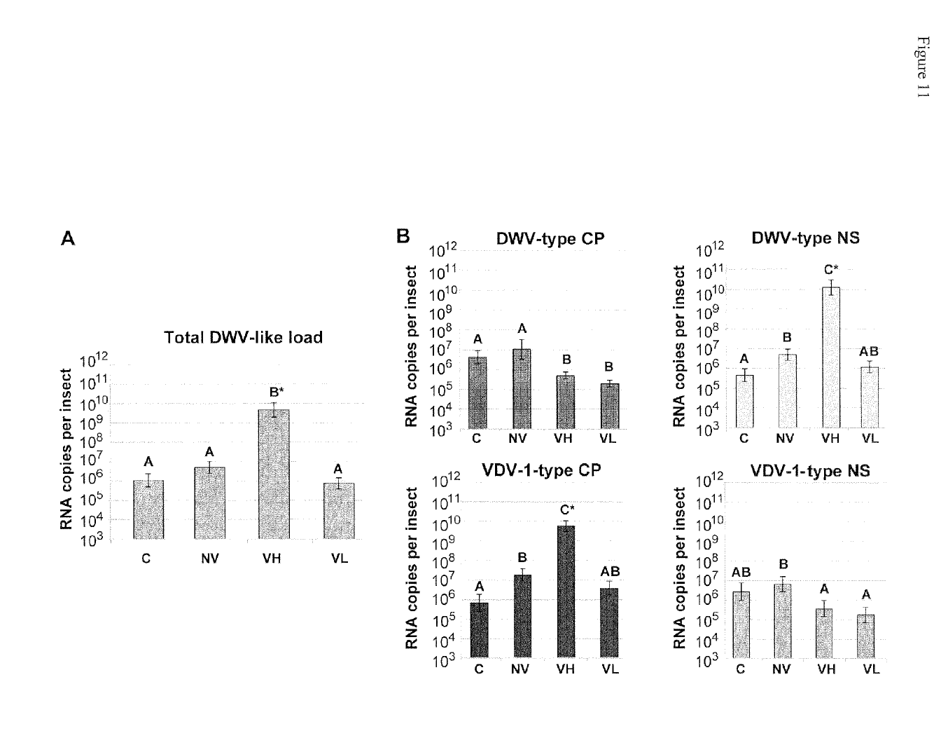

All RNA libraries analysed contained similar proportions of host-encoded miRNA reads, 12 to 18% of the total (Table 6), indicating both successful isolation of small RNA libraries and broad equivalence of the pooled sample sets. DWV- and VDV-1-specific siRNAs of both polarities were present in all treatment groups. DWV- and VDV-1-specific siRNAs could originate from either DWV or VDV-1, or from the previously reported [15] recombinants between these parental viruses (FIG. 10). Approximately 50% of all viral reads were 22 nt in length and 25% were 21 nt, with three to four times the number of sense orientation reads to antisense, irrespective of the read length (Table 6). To exclude variation due to the efficiency of library preparation, we normalised the siRNA number to the total number of honeybee miRNA reads in a library. The normalised loads of DWV/VDV-1-specific siRNA reads were similarly low in group C and the two NV group libraries (0.341, 0.377 and 0.397 siRNA per 1000 miRNAs respectively), ˜5 times higher in the two VL group libraries (1.926 and 2.066 siRNAs per 1000 miRNAs) which exhibited similar viral loads to groups C and NV (FIG. 11A, see below), but markedly higher in the VH group samples (285 and 287 siRNA per 1000 miRNAs; Table 6). The profiles of the DWV- and VDV-1-specific siRNA coverage of the DWV and VDV-1 reference genomes (FIG. 10) were most similar between groups VL and VH (Pearson correlation 0.955 to 0.963 for DWV, 0.945 to 0.962 for VDV-1, Table 7). The profiles for groups C and NV were more distinct from each other, and to VH or VL (Pearson correlation 0.593 to 0.786 for DWV, 0.399 to 0.726 for VDV-1; Table 7).

### Significant Changes in DWV Levels and Diversity Following Oral DWV Infection and Varroa Mite Feeding Revealed by Virus-Specific qRT PCR

We and others have previously reported changes in virus diversity at the population level [20] and the predominance of particular virus recombinants (i.e. a reduction in diversity) in honeybee pupae exhibiting high viral loads [15]. To quantify both viral load and diversity in individual honeybee pupae and their associated Varroa mites we used generic NS qRT-PCR primers or primer pairs specific for DWV or VDV-1 CP or NS coding regions (Table 1). We quantified the total virus levels (FIG. 11A) and the levels of the DWV-type and VDV-1-type CP and NS regions (FIG. 11B) in each of the 32 pupae as well as in each of 15 Varroa mite samples co-isolated with the VH and VL group pupae.

As already indicated (FIG. 2), the qRT-PCR Ct values used to separate the VH from the VL, NV and C experimental groups, indicated significant differences in viral loads in representative pupae (FIG. 11A), with the VH group exhibiting at least 3 log₁₀ higher levels of DWV-like viruses per pupa. When analysed using specific CP or NS primer pairs, the most pronounced difference was the increase in the number of genomes with the VDV-1 CP and the DWV NS sequences in the VH group pupae compared to the other treatment groups (FIG. 11B). In comparison with the control group C, the VH group exhibited a 6,000-fold increase in the VDV-1 CP region and a 26,000-fold increase in the DWV NS coding region. When compared with the NV group, the VH group showed lower relative increases (312-fold for VDV-1 CP and 2500-fold for DWV NS, P<0.0001 in both cases) because significant amplification of viruses bearing VDV-1 CP- (by 27-fold [P=0.0217]) and DWV NS-regions (10-fold [P=0.0314]) also occurred in the NV group relative to the control group C (FIG. 11B). The dramatic rise of the recombinant genome(s) containing VDV-1 CP and DWV NS in the VH group was also accompanied by a statistically significant 30-fold decrease (P=0.0151) of the DWV-type CP and 26-fold decrease (P=0.0477) of the VDV-1 NS compared with the NV group. The levels of VDV-1 CP and DWV NS coding regions showed strong positive correlation (r=0.9691) suggesting that this particular recombinant was preferentially acquired or amplified in the VH group pupae. The Varroa-exposed VL group also potentially acquired DWV from the mite as well as during larval feeding. It was therefore interesting to note that, when compared to the NV group, there was a statistically significant 54-fold decrease of DWV CP- (P=0.0057) and a 36-fold decrease of VDV-1 NS-regions (P=0.0151) in the VL group. Since both the VL and VH group pupae were mite-exposed but contained distinct levels and populations of DWV-like viruses we also characterised the viruses, and evidence of their replication, in the associated mites to determine if there was a correlation between high levels of virus in the honeybee and replication in the mite, as previously reported [38].

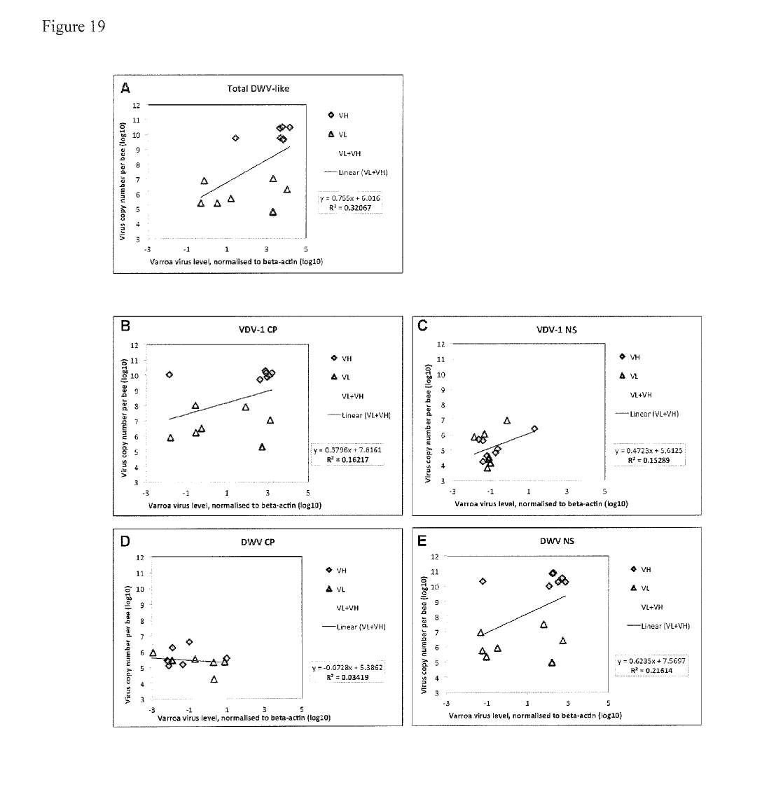

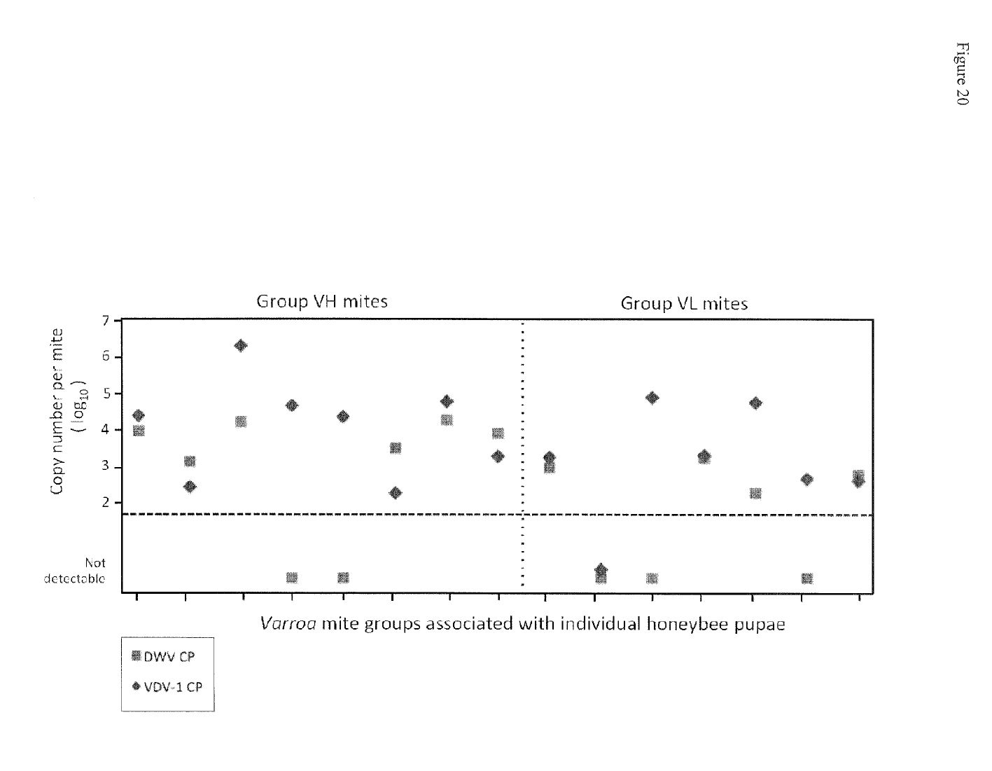

DWV- or VDV-1 specific qRT-PCR analysis demonstrated only a weak correlation with virus levels in the corresponding honeybee pupae (FIG. 19). The highest correlations were found for the total load of DWV-like viruses determined using universal NS primers across the VL and VH groups (r=0.567). Notably, we found that correlation between the levels of VDV-1 CP- and DWV NS-regions (sequences present in the predominant virus population in Varroa-infested VH group pupae; FIG. 19) in the Varroa mites and the bee pupae were lower, r=0.403 and r=0.465, respectively. We went on to investigate whether we could distinguish between the mite-associated viruses in the VL and VH groups on the basis of their ability to replicate (as determined by negative strand synthesis) in the ectoparasite. Negative strand RNA was generally low but detectable in 10/15 mites analysed, with no significant difference between the DWV or VDV-1 CP levels (FIG. 20). Together, these observations suggest that the low levels of DWV-like viruses in the VL group pupae cannot be explained by corresponding low levels of the virus in the mite and, similarly, that higher levels of the recombinant virus genomes in the mite-exposed honeybees (VH group) could not be attributed to either the preferential replication or absolute levels of these viruses in the associated mites.

### Virus Diversity is Markedly Reduced in Pupae but not in the Associated Varroa Mites

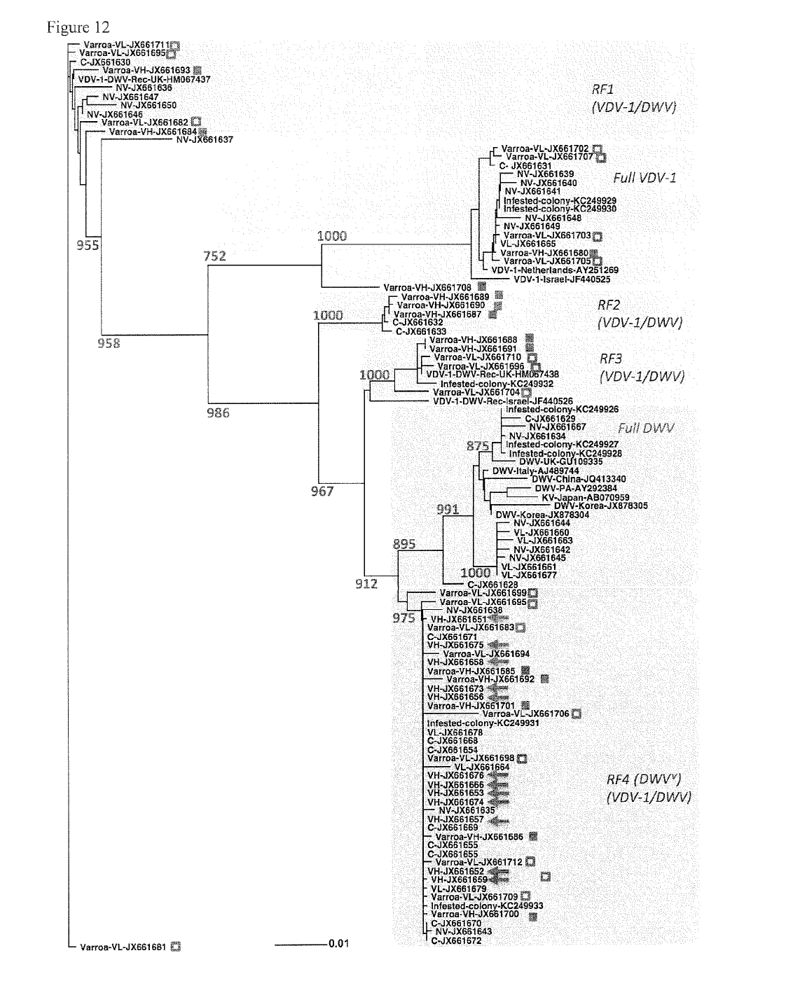

The dominance of recombinant viruses bearing VDV-1 CP and DWV NS coding regions in the VH group was strongly suggested by qRT-PCR (FIG. 11B). Since recent studies have demonstrated that mite infestation is associated with a marked reduction in virus diversity at the regional scale [20], we extended our analysis to determine DWV-like virus diversity in individual pupae of the four exposure groups and, where appropriate, the co-isolated mites. In parallel, we also sampled random purple-eye stage pupae from the Varroa-infested colony to determine the pre-existing virus population at frame transfer. Nested PCR using generic (outer) and four specific (inner) primer pairs (Tables 1 and 5)—for each possible combination of CP and NS region—was used to amplify a 1.3 kb fragment spanning a central region of the virus genome (corresponding to nucleotides 4926-6255 of the DWV genome; GenBank accession No. AJ489744) containing both CP and NS coding regions. We noted that no recombinants bearing a DWV CP region and VDV-1 NS region were detected in any of the experimental groups. For each of the eight pupae from the four exposure groups (C, NV, VL, VH), and pupae-associated individual mites from the VL and VH groups, PCR fragments were cloned and 8-18 individual clones sequenced. In total, 93 individual sequences were obtained of the 1330 nt. region and aligned with 12 DWV-like sequences (DWV, VDV-1, KV and recombinants thereof; see Materials and Methods) to generate a robust phylogenetic tree (FIG. 12) due to the 22.71% sequence divergence in the region analysed.

The resulting dendrogram contained six distinct clusters, one each for non-recombinant DWV- or VDV-1-like sequences, together with four different VDV-1/DWV recombinant forms (designated RF1-RF4; FIG. 12). Individual sequences obtained from pupae in exposure groups C, NV, VL and the Varroa-infested colony were present in all the major clusters indicating that these contain a significant diversity of viruses. In striking contrast, viral sequences from the VH experimental group exhibited almost no sequence divergence (0.15% at the nucleotide level), and consequently all clustered within a single clade (designated VDV-1/DWV RF4 in FIG. 12). Therefore, the reduction in viral diversity (as previously determined by high resolution melting analysis) associated with the introduction of Varroa observed at the scale of tens of colonies exposed to the mite over several years [20] is reflected at the level of individual honeybee pupae following exposure to Varroa for 6 days.

One interpretation of the near-clonality of viral sequences in the VH group was that these were the only ones carried, and hence transmitted, by the mite. However, with the exception of the non-recombinant DWV cluster, which was not detected in the mite, the 32 viral sequences obtained from Varroa were widely distributed within the dendrogram (open symbols in FIG. 12). These results imply that, with the possible exception of non-recombinant DWV, Varroa is capable of acquiring and maintaining a diversity of DWV-like viruses, but that—either during or following transmission to naïve pupae—only a subset of these (RF4 in FIG. 12) are amplified to the very high levels observed in the VH group. Since the obvious difference between the horizontal transmission of DWV per os (larval feeding) and by Varroa is that the latter involves direct inoculation of virus to the haemolymph in pupae we investigated the recapitulation of this process by direct injection of pupae in vitro.

Inoculation of Honeybees by Injection into Haemolymph Results in Preferential Amplification of Specific VDV-1/DWV Recombinants

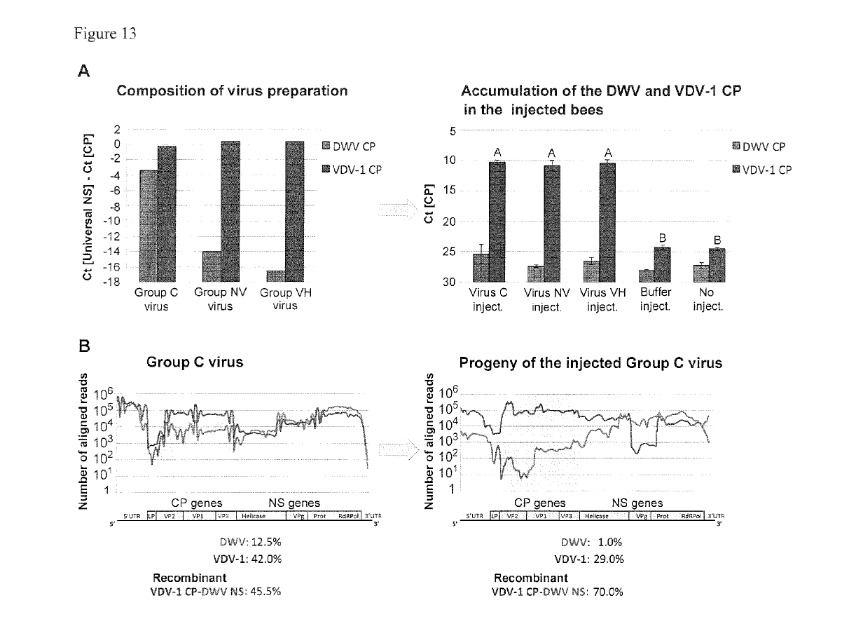

We directly injected white eye pupae (day 12-13 of development) maintained in vitro (as described in [39]) with virus particles purified from groups C, NV and VH pupae as described previously [15]. As before, we determined the proportion of the DWV- and VDV-1-type CP coding regions in the inocula and injected pupae (following incubation to the purple-eye stage for 3 days) by qRT-PCR using strain-specific primers to the CP and universal primers to the NS region. Virus preparations from groups NV and VH contained higher and broadly similar levels of VDV-1-like CP coding regions. The amount of DWV-like CP coding regions was much higher in the virus preparation from the group C pupae (where it accounted for ˜12% of the population) than from either the NV or VH group pupae (FIG. 13A). Pupae inoculated with buffer alone exhibited no significant increased accumulation of DWV-like viruses when compared with untreated pupae (FIG. 13A). In striking contrast, irrespective of the source of viral inocula, pupae directly injected with virus preparations exhibited high virus levels characterised by markedly amplified VDV-1-like CP coding regions when compared to DWV-like CP sequences (FIG. 13A). Directly injected pupae were therefore similar, in both DWV-like virus levels and identity, to those previously observed in the VH experimental group (FIG. 11A and FIG. 2).

We additionally conducted next generation sequencing (Illumina paired-end reads) to comprehensively characterise the group C inocula and the viruses present in pupae injected with the group C virus. The composition of the inoculum, as determined by qRT-PCR and subsequent MosaicSolver [40] analysis of the NGS reads, were in close agreement and consisted of 12.5% DWV (in excellent agreement with the qRT-PCR-determined level, see above), 42% VDV-1 with the remainder being VDV-1 CP-encoding recombinants with a DWV-like NS region (FIG. 13B). Three days after injection, the pupae inoculated with group C virus exhibited a marked reduction in the DWV content (from >12% to 1%) and a concomitant increase in recombinant forms of the virus (70% of the total) that were characterised by the presence of VDV-1 CP coding region and DWV-like NS regions (FIG. 13B). These results further support our previous conclusion that DWV-like viruses bearing VDV-1 CP coding regions, and particularly recombinant forms with DWV-derived NS coding regions [15], have a selective advantage in Varroa-infested honeybee colonies, and additionally indicate that this advantage is manifest after transmission of the virus by direct inoculation and is not dependent upon Varroa per se.

Independent Verification of DWV Diversity Reduction by Deep Sequencing of the Honeybees from a Varroa-Infested Colony.

The sequence analysis of DWV in Varroa-exposed pupae (FIG. 12) in the frame-transfer study supported the presence of a single, near-clonal, recombinant form of the virus in VH group honeybees. To formally exclude a role for PCR-biased amplification in this result and to extend our analysis to investigate virus diversity in independent samples (geographically and temporally), including asymptomatic and symptomatic newly emerged workers, we investigated virus diversity using next generation sequencing (NGS). We sampled individual adult nurse worker bees, both asymptomatic and exhibiting the obvious wing deformities and abdominal stunting characteristic of DWV disease, from a naturally Varroa infested colony. We additionally investigated virus diversity in purple-eye stage pupae to which we had injected (at the white-eyed stage 3 days previously) virus purified from pupae from the same colony a month earlier. Analysis was conducted on individual pupae using a high-throughput RNA-seq approach [41] with an mRNA protocol which allowed unbiased detection and quantification of all poly(A) containing RNA, this would include both host mRNA and the polyadenylated DWV-like genomic RNA [12].

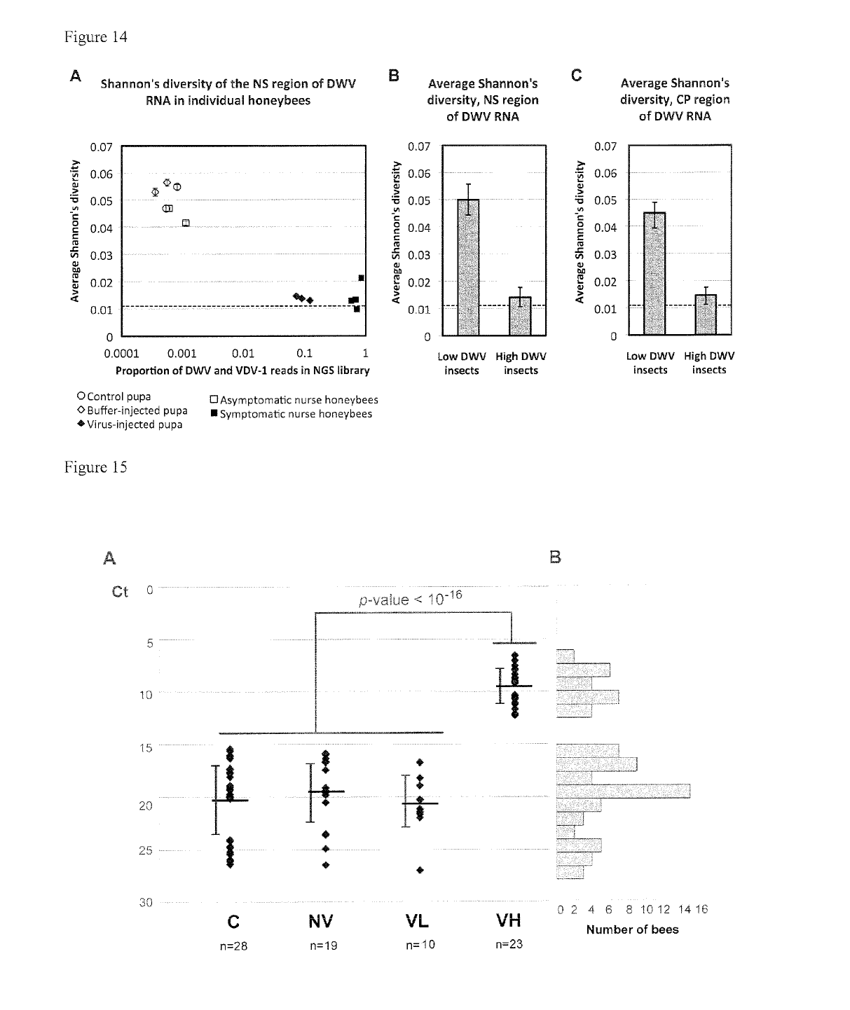

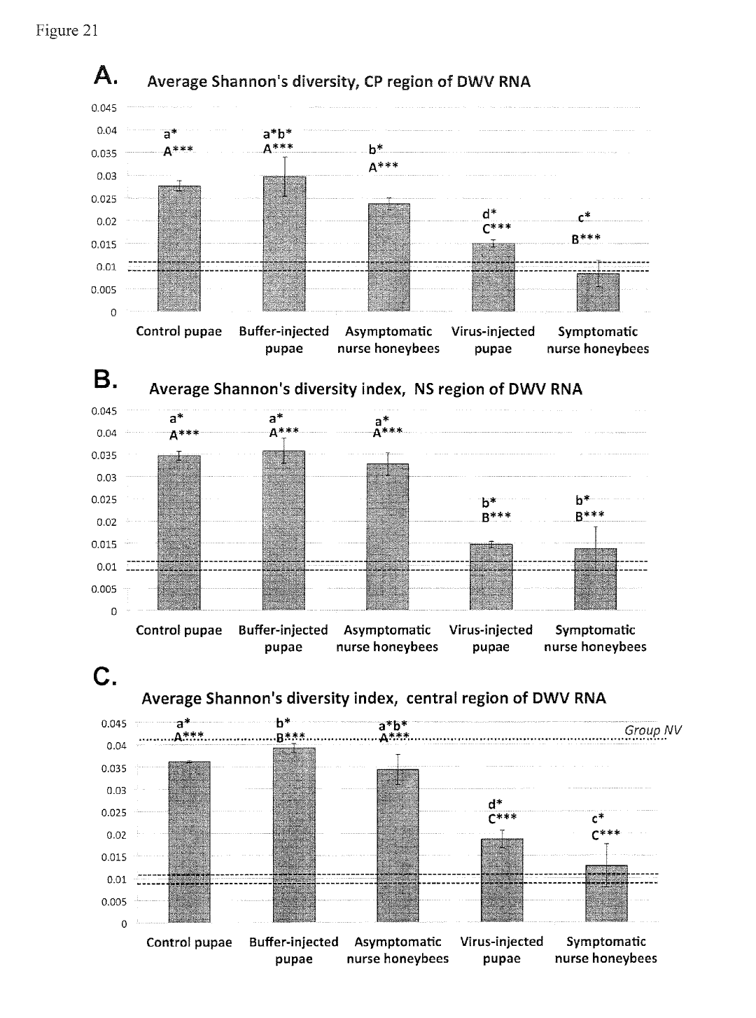

The NGS reads were aligned to reference DWV and VDV-1 sequences (GenBank Accession numbers GU109335 and AY251269 respectively), and the pileup profiles were analysed. The proportions of DWV and VDV-1 reads in the libraries (each containing about 10 million reads) showed a bimodal distribution and were either very high (from 7.41% to 83.87%, FIG. 14A horizontal axis) for injected pupae and symptomatic nurse bees, or about a thousand fold lower (0.04% to 0.11%) for Varroa-naïve control pupae, for pupae inoculated with buffer alone and for asymptomatic nurse bees from the Varroa-infested colony (FIG. 14A, Table 4). The remaining reads were of honeybee mRNAs. Distribution of the reads with similarity to DWV and VDV-1 suggested that all samples, irrespective of viral load, contained recombinant viruses with the CP derived form VDV-1 and NS region derived from DWV, as described above and in previous studies [15,16]. To assess virus diversity, we calculated Shannon's diversity index [42] for the aligned NGS reads from each experimental pupa or adult bee. Despite the ubiquitous presence of recombinant DWV-like genomes (all consisting of a VDV-1 capsid and DWV non-structural coding regions) there was a striking reduction of virus diversity in the bees and pupae exhibiting high virus loads (FIG. 14A,B,C). Average Shannon's diversity index for the NS and CP regions of the viral genomic RNA were significantly higher in the samples tested with low virus levels compared to those with high virus levels (0.1% level Fishers LSD test). At the same time, we observed no significant differences in Shannon's diversity index for NS and CP regions at the 5% level (Fisher LSD test) within the low virus group which consisted of Varroa-naïve control pupae, pupae injected with buffer alone, and asymptomatic nurse bees from a Varroa-infested colony (FIG. 21A, B). For the samples tested with high virus levels (pupae injected with virus in vitro and symptomatic nurse bees from a Varroa-infested colony), no differences were observed at the 5% level (Fisher LSD test) for the NS region. Indeed, combined low virus level and high virus level groups showed significant differences in Shannon's diversity index values for the CP and NS regions even at the 0.1% level (FIG. 14B,C). For comparison we determined Shannon's diversity index for a sample prepared by in vitro transcription of two full-length DWV cDNA clones, GenBank accession numbers HM067437 and HM067438 [15], mixed, post transcription, at a known ratio and used as a template for NGS. We additionally used this control sample to determine the component of the observed diversity that was attributable to NGS sequencing errors which we quantified at about 0.5%, similar to that previously reported [43]. We calculated the threshold Shannon's diversity, a 95% confidence limit for clonal input RNA library (shown as dashed line in FIG. 14A) using the approach described in [44]. Remarkably, while the diversity index of all samples with low DWV levels (control and buffer-injected pupae, and asymptomatic nurse honeybees) were well above this threshold, diversity values of samples with high DWV levels (virus-injected pupae and symptomatic nurse honeybees) were either very close or below this clonality threshold. Similar results were obtained when diversity was estimated using multiple sampling as described in Material and Methods (FIG. 21). In this case the clonality threshold value (the range shown with the dotted lines in FIG. 21) was also almost indistinguishable from the diversity present in symptomatic nurse bees from the Varroa-infested colony indicating that the diversity in these honeybee samples was close to the limit of detection using NGS analysis. This reinforces the near-clonal nature of the virus population in Varroa-exposed symptomatic nurse bees and is in good agreement with the sequence analysis of VH group pupae following PCR amplification of the central region of the virus genome (FIG. 12). To further explore the near-clonal nature of the virus population in symptomatic nurse bees from a Varroa-infested colony we used a pair of flanking primers to the DWV-like genomic RNA to amplify and clone full-length viral cDNAs from these samples (GenBank accession number KJ437447). The central 1330 nt. region of this clone was identical to that previously characterised from VH group pupae (FIG. 12) despite being sampled from a separate colony in a different apiary over two years later. The consensus viral sequences, which were assembled from the NGS libraries from symptomatic honeybees with high DWV levels, showed highest overall identity with the full-length clone KJ437447 (specifically 99.15% [SD=0.31%] nucleotide and 99.78% [SD=0.09%] amino acid identity) and the 1330 nt. sequences from VH group pupae, e.g. JX661656 (98.84% [SD=0.57%] nucleotide and 100.00% amino acid identity; Table 4). In respect to the samples with low DWV levels, we found that Shannon's diversity index for the NV group sequences (Varroa-free orally infected pupae; FIG. 12) was 0.04172. This value was very close to the Shannon's index values in the same genomic region for the pupae exhibiting low virus levels from the Varroa-infested hive, 0.03623, SD=0.00026, for control (i.e. not injected) pupae (0.03929, SD=0.00097) and for pupae injected with buffer alone (0.03929, SD=0.00097); FIG. 21 C).

### Discussion

The introduction and global spread of the parasitic mite Varroa destructor in honeybees (Apis mellifera) has had significant impact on the health and survival of infested colonies [8,13]. Colony losses associated with Varroa are predominantly attributed to the RNA virus payload vectored by the parasite and transmitted when the mite feeds on honeybee haemolymph [10,45]. Although Varroa were reported to vector at least 5 RNA viruses, the picoma-like Iflavirus deformed wing virus (DWV) is of particular interest and importance; deformed wing disease is associated with mite infestation [9] and high levels of DWV exacerbate overwintering colony losses [10]. Furthermore, of the viruses analysed, only DWV levels increased upon introduction of Varroa to Hawaii [20]. Here we show that virus levels exhibited a bimodal distribution in developing honeybee pupae—low in the absence but generally high following mite exposure—with high viral levels associated with emergence of a distinct recombinant form of the virus. This complements observations of the effects of the introduction of Varroa at the regional scale (e.g. Hawaii), which is associated with a dramatic reduction in DWV variation and the emergence of dominant strains over a scale of months or years [20]. Our study also strongly suggests that DWV is widespread and present in all UK honeybees, in Varroa-free and Varroa-infested colonies. By using different molecular approaches including RNA-seq, cloned cDNA sequencing and qRT-PCR, we demonstrated that, contrary to the recently reported figure of 36% DWV presence in the UK honeybees [46], DWV was present in all n=250 tested honeybees, including those from a Varroa-free region (n=47) (FIG. 15 and data not shown). Striking differences in reported DWV incidence might be due to differential sensitivity of the primers used in these studies, especially in the honeybees with low DWV levels, which have higher genetic diversity (FIGS. 12, 14).

To better understand the interactions of the honeybee host, Varroa and DWV that account for the observed emergence of presumed pathogenic, near-clonal strains of the virus we analysed events at the level of individual honeybee pupae. We reasoned that the large scale global, temporal and population changes observed reflect the cumulative outcome of interactions that occur at the level of individual pupae within the colony. We further reasoned that analysis at this level would allow two hypotheses accounting for the high levels of overt deformed wing disease beekeepers associate with heavy Varroa infestation to be tested. These hypotheses, themselves not mutually exclusive, are that; a) Varroa suppresses the antiviral honeybee defences so allowing unrestricted DWV replication and b) the presence of Varroa results in the selection and transmission of particular pathogenic variants of DWV, resulting in serial amplification within a mite infested colony. We tested these hypotheses by transferring honeybee larvae from a Varroa-free to an experimental Varroa-infested environment, stratified pupae of a standardised age in terms of mite-exposure and viral load, and investigated transcriptome and RNAi responses of the host and the virus population in individual mites and associated pupae.

We demonstrated that Varroa-naïve honeybee larvae (group C) transferred into a Varroa-infested colony (effectively mimicking the exposure to oral and mite-transmitted DWV during larval and pupal development) can, after incubation for 6 days, be stratified into three distinct pupal groups by Varroa exposure (presence or absence) and DWV level (high or low). High virus levels were not observed in the absence of Varroa. Group NV comprised pupae from capped cells free of Varroa. As Varroa enters the cell immediately prior to capping [27], we assume this group only acquired viruses per os during larval feeding. The VH and VL groups contained Varroa within the capped cell with evidence of Varroa feeding on the pupae—including nymphal forms present and signs of abdominal piercing. These groups of pupae harbour strikingly different DWV populations: the VL group having low viral levels and high diversity that are not significantly different from the C and NV groups, whereas the VH group carries 1,000-10,000 times the viral loads of a single phylogenetic type (FIG. 12). We compared the transcriptome and virus-specific siRNA pool between the four exposure groups, the virus level and diversity in associated mites and determined the consequences of direct virus injection to experimentally test the two proposed hypotheses accounting for the observed dominance of particular virulent strains of DWV in the presence of Varroa. We additionally characterised virus sequence and diversity in injected pupae and independent naturally Varroa-infested colonies.

### Transcriptome Changes in Response to DWV and Varroa Mite Feeding

Previous analyses of transcriptome or immune response changes in Varroa-exposed honeybees have produced contradictory results, perhaps due to the analysis of pooled individuals and/or pupae of different ages. These confounding influences may have obscured the marked changes in gene expression that we observed in response to either mite or viral pathogens, as emphasised by the transcriptome differences observed in the Varroa-associated pupae in groups VL and VH, which respectively exhibited 493 (˜5% of transcriptome) and 951 (˜9%) significantly differentially expressed (DE) genes when compared with the control group C (FIG. 9A). By stratifying Varroa-exposed pupae by viral load we can provisionally define transcriptome changes resulting from mite-associated activities such as wounding, feeding and exposure to salivary peptides (the 444 genes shared by VL and VH groups) and those triggered by the high viral load (>3 log₁₀ higher in VH than VL; FIG. 9A). We acknowledge that the C to VL contrast may include genes involved in suppressing high levels of mite-transmitted DWV accumulation, an interpretation that warrants further study. The NV, VL and VH pupal groups also acquired DWV during larval feeding in the Varroa-infested colony, which on account of the preferential amplification of particular recombinant forms of DWV (FIG. 11B, discussed further below and [15]) contains a distinct virus population, the composition of which differs from historically Varroa-free control colonies. Transcriptome comparison between the C and NV groups showed significant changes in a large number of genes (416, ˜4% of transcriptome), many of which were also altered in the C to VL (220) and C to VH (385) contrasts (FIG. 9A, B). These may reflect per os exposure, and the resulting responses to the particular virus population circulating in the Varroa-infested hive, which resulted in changes of DWV strain composition in NV compared to C (FIG. 11B), together with changes resulting from environmental differences (such as circulating pheromone) between the originating and test colonies which would be common to all three exposure groups. The set of 59 genes DE in the contrast NV to VL (exposure to Varroa feeding at the pupal stage which did not result in elevated viral loads), was largely different from the DE genes in contrast C to NV with only one gene shared. At the same time, the NV to VL set showed high commonality with the DE genes in the contrasts C to VH, C to VL, and NV to VH (34, 51, and 27 genes respectively; FIG. 9A, B). Observed DE gene commonality in the contrasts was consistent with an orthogonal expression pattern (FIG. 9B) following treatments (FIG. 9), with different sets of genes DE in response to per os infection (C to NV), exposure to mite feeding (NV to VL), and high DWV (VL to VH) (FIG. 9B).

Gene Ontology (GO) analysis gave additional insights into the transcriptional responses in honeybees following experimental treatments (FIG. 9). It has been demonstrated that genes associated with the same GO terms are likely to have the same transcription factors binding to their promoter regions, which may result in co-regulated expression of these gene sets [47]. Therefore, statistically significant overrepresentation of GO BP terms associated with the DE genes may suggest coordinated and distinct honeybee responses to per os exposure (C to NV contrast) and to Varroa and/or Varroa-injected virus (NV to VL contrast) (FIG. 11A, FIG. 15). Such coordinated transcriptional responses may include genes involved in suppression of virus replication in the NV and VL group pupae (FIG. 15), which will require further analysis. In contrast to this situation, the transcriptional changes specifically associated with the increased virus levels in group VH (VL to VH contrast) had no significantly over- or under-represented GO BP terms (FIG. 9A). This suggests that honeybees did not respond in a coordinated manner to the increased virus load, and that presumably unrestricted DWV replication caused dysregulation of transcription and/or mRNA stability in the honeybee similar to that previously reported in picornavirus infection of mammalian cells [48-50].

### Changes in Expression of Immune-Related Genes

We analysed changes in expression of known and presumed immune-related genes (Table 3, FIG. 18) defined previously [34,35] and by gene ontology (GO) terms associated with Drosophila homologs [33]. In particular, a number of proposed components of the Toll signalling pathway were affected, although the lack of activation of the antimicrobial peptide genes suggested that no activation of the Toll and Imd pathways had occurred [34,35,51]. In contrast to both the Varroa-exposed groups (VL and VH) the NV group was the only group in which there were more up- than down-regulated immune-related genes when compared with the control (Table 3, FIG. 18). The majority of the changes seen in the C to NV contrast were also seen in the groups that acquired DWV both orally and via Varroa (C to VH, C to VL contrasts), implying that Varroa exposure may exert a dominant immunosuppressive influence over any up-regulation observed following oral exposure alone. Significantly enhanced expression of the honeybee orthologue of Vago (GB10896; Table 3), a secreted protein upregulated in Drosophila and Aedes following detection of viral dsRNA by Dicer during virus infection [52,53], was observed in all groups exposed to oral DWV in the Varroa-infested colony (NV, VL and VH) when compared with group C.

Varroa exposure (VL or VH groups) resulted in down-regulation of several putative components of the honeybee Toll signalling pathway [51], including two Toll receptor orthologs (GB10640, GB17781), CLIP-domain protease spirit (GB14044) and the Toll receptor ligand spatzle (GB15688). In addition, spatzle was down-regulated when the VH group was compared against the other experimental groups, suggesting down-regulation of this gene may be a response to the elevated virus levels in the group VH, rather than the presence of Varroa per se. Toll signaling pathways are implicated in antiviral resistance to the RNA virus Drosophila virus X [54], possibly controlling proliferation of haemocytes which, because of their involvement in phagocytosis, play a central role in insect immunity [54,55]. We also observed down-regulation of a Tetraspanin 68C (Tsp68C) ortholog (GB16002, GB13670), a cell surface membrane scaffolding protein previously implicated in receptor modulation during hemopoiesis [56], an ortholog of pannier (GB 19895), a GATA transcription factor required for differentiation of plasmatocytes (which resemble the mammalian macrophage lineage [57]), and a serrate ortholog (GB15155), a membrane ligand for the Notch receptor implicated in differentiation of haemocyte-related crystal cell precursors which function in pathogen defence via melanisation [58]. These transcriptome changes may help explain functional studies in which salivary secretions from Varroa mites damage moth caterpillar haemocytes [59] and suggest that Varroa-mediated depletion of haemocytes, a key component of the immune response of insects [60-62], may contribute to enhanced susceptibility to DWV and other viruses. Interestingly, we also observed suppression of the Friends-of-GATA transcription factor U-shaped (ush) ortholog (GB 16457), in the C to VL and NV to VL contrasts. Drosophila ush is reported to antagonise crystal cell development [63,64], implying that the low level of virus accumulation in the VL group may be due to elevated numbers of crystal cells resulting from ush down-regulation.

Although by definition descriptive, transcriptome analysis of pupae stratified according to Varroa and virus-exposure, also provides direct insights into possible pathogenic mechanisms. In the contrast C to VH we observed differential expression of orthologs of five Drosophila homeobox genes (summarised in Table 8) encoding transcription factors which are involved in insect development [65]. Most of these DE genes are reported to be expressed at early pupal stages and involved in abdomen (Abdominal B), appendage (apterous) or brain development (extradenticle). This may explain previously reported developmental abnormalities in the honeybee that are associated with high DWV levels at the pupal stage [13] and warrant further investigation to potentially determine the molecular mechanism underlying DWV pathogenesis.

### RNAi Responses to DWV in Varroa-Infested Pupae

Notwithstanding the absence of significant changes in gene expression of key components, such as Dicer and Argonaute, of the RNAi response—the major antiviral mechanism in insects [25]—we explored the relationship between DWV-like virus levels and the corresponding siRNA populations. In particular, we sought to investigate if high levels of DWV in VH honeybees was associated with the limited accumulation of virus-derived siRNA, implying the virus may express an siRNA suppressor as, for example, demonstrated in Alphavirus infection of mosquitos [66]. Although DWV- and VDV-1 specific siRNAs were recently detected in adult honeybees [67,68], these studies could not show if RNAi is involved in suppression of the virus, because viral genomic RNA levels were not quantified. Analysis of siRNAs in the honeybees of the frame transfer experiment showed that the predominant DWV- and VDV-1-specific siRNAs were 22 nt in length with genome sense strand-specific siRNAs present at a 3-4 fold excess over antisense. This was consistent both with the presence of replicating DWV-like viruses in all experimental groups and with the known activity of Dicer in other insects [25] and strongly suggests normal functioning of Dicer in honeybees [69]. As insects do not amplify siRNA populations [25], it was unsurprising that virus-specific siRNA levels were broadly proportional to the level of viral genomic RNA determined by qRT-PCR; the C and NV groups exhibited ˜10³ times less viral genomic RNA than the VH group and normalised siRNA levels differed by ˜770 times (Table 6). The exception to this was the siRNA response in the VL group which was ˜5 times higher proportionally than the level of VL virus genomic RNA (Table 6). The relationship between the levels and compositions of the viral genomic RNA and virus-derived siRNA may be altered by differences in targeting of the individual components of DWV-like virus population by the honeybee RNAi machinery, as observed during West Nile virus infection of mosquitos [70]. Although the presence of virus-specific siRNAs does not necessarily correlate with effective silencing—viruses may encode late-acting suppressors such as the Argonaute-inhibiting 1A protein of cricket paralysis virus [71]—the robust siRNA response in the VL group may contribute to suppression of DWV replication and the differences between this response and that observed in the VH group may be a fruitful area for further analysis.

Genetic Diversity of the DWV Population is Determined by Route of Transmission Rather than Preferential Amplification of Virus in Varroa

The introduction of the parasitic Varroa mite elevates the level of DWV-like viruses [20], amplifies particular recombinant forms (RF) bearing the capsid determinants of VDV-1 and non-structural genome region from DWV [15,16] and dramatically reduces the diversity of DWV-like viruses in a population [20]. Using complementary approaches including strain-specific qRT-PCR and sequencing together with next generation sequencing of the virus genome and host siRNA response to infection, we analysed individual pupae exposed to DWV during larval feeding and following mite exposure, and recapitulated horizontal transmission of virus by Varroa using direct injection.

The C, NV and VL exposure groups all carried low viral loads and exhibited high virus diversity (FIG. 11B, FIG. 12). However, the virus populations carried were distinct, with the NV and VL experimental groups containing a diverse range of recombinant forms of DWV-like viruses bearing the capsid coding region of VDV-1 and the non-structural coding regions of DWV. In contrast, the VH group exhibited very high levels of a specific near-clonal (0.15% divergence in the regions sequenced) recombinant form of DWV (labelled RF4 in FIG. 12). Due to the subsequent identification of the same near-clonal virulent virus in temporally and spatially distinct samples (see below) we henceforth designate this virus DWV^{V} to discriminate it from other circulating recombinants forms. This suggests that the changes reported in virus levels and diversity at a regional scale [20] reflect events occurring within a few days (uncapped to the purple-eye stage) in individual mite-exposed pupae. Nearly identical, clustering tightly within the DWV^{V} clade, were also detected in pupae from the C, NV and VL groups (FIG. 12). Since these groups have significantly lower viral loads it implies that the high viral loads seen in the VH group cannot be solely attributed to their infection with a particular recombinant form of the virus.

We reasoned that there were two possibilities that might account for the marked amplification of DWV^{V} in the VH group pupae. Firstly, the mite may have delivered a high dose of one specific recombinant form, perhaps reflecting its preferential replication in the ectoparasite. Secondly, we considered that DWV^{V} might have a growth advantage when inoculated into haemolymph by Varroa (potentially in addition to the preferential amplification in the mite). To distinguish between these possibilities we sequenced qRT-PCR amplified viral RNA from mites co-isolated from capped cells containing group VL and VH pupae. We also investigated the consequences of Varroa-independent mechanical virus transmission by direct injection of mixed virus preparations to Varroa-naïve pupae and subsequent monitoring of virus levels and diversity.

Individual Varroa mites contained a diversity of DWV-like sequences that were well distributed throughout the phylogenetic tree of virus sequences from pupae (square symbols, FIG. 12). Using VDV-1- and/or DWV-specific primer pairs spanning the central 1.3 kb region of the virus genome mites were detected containing VDV-1 and all four distinct RFs identified in the four experimental groups in the frame transfer study. Only non-recombinant DWV was absent from the 32 mite-associated viruses sequenced. We also detected negative strand sequences of both DWV and VDV-1 CP regions in the majority of the 15 mites tested (FIG. 20), implying that virus replication does occur in the mite. Although we did not detect DWV CP among the central 1.3 kb region sequences amplified from the mites (FIG. 12), this could be a consequence of limited experimental sampling and the higher levels of VDV-1 CP in the population, a conclusion supported by analysis of the negative strands present (FIG. 20).

Although further studies will be required to determine whether sampling stochasticity accounts for the apparent absence of non-recombinant DWV in Varroa, together these results suggest that at least at the level of the entire mite—there is no selection, either by absolute presence or replication capability, for the DWV^{V} RF that accumulates to high levels after mite exposure in VH group pupae.

Since the diversity of virus present in Varroa indicates that the near-clonal virus population in the VH group is not due to the mite delivering either a restricted virus type or to elevated levels of DWV^{V} in the mite we went on to inoculate pupae with a mixed virus population prepared from group C pupae and characterised the resulting virus population after three days. Recipient purple-eye stage pupae contained high virus loads which, compared with the inocula, had markedly reduced levels of DWV-like virus and elevated levels of a VDV-1/DWV recombinant (FIG. 13). Although, the resulting virus diversity was not as limited as seen in the naturally infected VH group, we attribye this to the restricted incubation time between inoculation and sampling (3 days vs. 6 days), in part imposed by experimental limitations of working with late-stage larvae and early-stage pupae which are vulnerable to handling damage. Despite these limitations, these results clearly demonstrate that direct inoculation of a mixed virus preparation, recapitulating virus inoculation by the mite, results in a marked reduction in virus diversity. We additionally demonstrated, by RNA-seq analysis of temporally and geographically independent symptomatic nurse bees and similarly independent pupae directly injected with virus preparations, that essentially the same near-clonal virus (DWV^{V}) was present as previously identified in the VH group pupae. In parallel, control asymptomatic nurse bees or mock-injected pupae exhibited high diversity and low levels of virus (FIG. 7, FIG. 21, Table 4), as previously seen in the C and NV groups during the frame transfer study. The unselective RNA-seq methodology excludes the possibility that virus clonality at high virus loads was a consequence of PCR bias. The remarkable restriction in virus diversity in both injected pupae or symptomatic nurse bees exhibiting high viral loads was in good agreement with that seen in group VH pupae (FIG. 12) determined following qRT-PCR amplification (0.15% diversity).

We propose that the strikingly elevated levels and associated restricted diversity of DWV^{V} (RF4-type; FIG. 12) in both the Varroa-exposed VH group pupae and characteristically DWV symptomatic nurse bees is because this virus has a preferential advantage when delivered directly to haemolymph of developing pupae. There remains the possibility that DWV^{V} alone replicates to elevated levels in the salivary glands of Varroa and is the only DWV-like virus transmitted during feeding. However, we do not favour this hypothesis as we would expect it to result in DWV^{V} being the predominant virus detected when whole-mite RNA samples were analysed. Furthermore, we also present evidence (FIG. 14A, Table 4) that DWV^{V} predominates when a mixed virus population is directly inoculated. Nevertheless, it remains an intriguing avenue for further study. Assuming this is not the explanation, the molecular mechanisms underpinning the advantage of the near-clonal DWV^{V}, be it evasion of the host antiviral response, specific tissue tropism or some other aspect of the virus-host interaction, will require further studies. This will necessitate immunohistological analysis of orally infected or injected pupae, the development of a reverse genetic system to identify determinants of DWV tropism, and the analysis of the contribution of immune-related (and other) host genes using RNAi-based strategies [72,73].

TABLE 3
Differential expression of the honeybee immune-related genes in response to oral DWV and Varroa mite feeding.

| | | | | Fold change in contrast ^{e} | | | | | |
|---|---|---|---|---|---|---|---|---|---|
| | | | | C | C | C | NV | NV | VL |
| BeeBase | Drosophila | | Pathway, | to | to | to | to | to | to |
| ID ^{a} | homolog ID ^{b} | Gene ^{c} | category ^{d} | NV | VH | VL | VH | VL | VH |
| GB10896 | FBgn0051997 | Vago | antivir | 1.860 | 1.468 | 1.833 | — | — | — |
| GB10640 | FBgn0029114 | Tollo | Toll | — | −0.452 | — | — | — | — |
| GB17781 | FBgn0036494 | Toll | Toll | — | −0.215 | — | — | — | — |
| | | | Toll | — | | — | — | — | — |
| GB15688 | FBgn0003495 | spatzle | | | −0.381 | | 0.249 | | 0.346 |
| | | | | — | | — | — | — | — |
| GB17879 | FBgn0030310 | PGRP-S3 | Toll | | −0.564 | | 0.381 | | 0.381 |
| GB14044 | FBgn0030051 | spirit | Toll | — | −0.701 | — | — | — | — |
| GB19582 | FBgn0028984 | NEC-like | Toll | 0.482 | 0.642 | 0.475 | — | — | — |
| | | | Toll | | | — | — | — | — |
| GB13935 | FBgn0261988 | Gprk2 | | — | −0.322 | 0.311 | 0.323 | 0.312 | |
| GB19452 | FBgn0243514 | GNBP3 | Toll | 0.800 | 1.599 | 0.931 | — | — | — |
| | | | Toll | — | | — | — | — | — |
| GB19961 | FBgn0040323 | GNBP1 | | | −0.400 | 0.289 | | | |
| GB19066 | FBgn0260632 | dorsal | Toll | — | 0.202 | — | — | — | — |
| | | | | — | | — | — | — | — |
| GB17654 | not found | SP45 | SP | | −0.535 | 0.669 | | | |
| | | | | — | | | — | — | |
| GB16214 | FBgn0036287 | SP38 | SP | | −0.214 | — | 0.284 | 0.236 | — |
| GB14309 | FBgn0033359 | SP33 | SP | 1.138 | 1.277 | 1.153 | — | — | — |
| GB11511 | FBgn0038595 | SP32 | SP | 1.667 | — | 1.395 | — | — | — |
| GB11743 | FBgn0035290 | AmSCR | Scav | 0.806 | 0.511 | — | — | — | — |
| | | | | — | | — | — | — | — |
| GB10506 | FBgn0058006 | AmSCR | Scav | 0.359 | −0.369 | 0.350 | | | |
| | | | | | | — | — | — | — |
| GB15155 | FBgn0004197 | Serrate | Notch | — | −0.422 | 0.538 | | 0.324 | |
| | | | | | | — | — | — | — |
| GB13135 | FBgn0014020 | Rho1 | JNK | — | −0.343 | 0.314 | | | |
| | | | | — | | — | — | — | — |
| GB12838 | FBgn0015286 | Ras | JNK | 0.217 | −0.302 | | | | |
| GB19901 | FBgn0243512 | puckered | JNK | 0.600 | 0.441 | — | — | — | — |
| GB12212 | FBgn0001297 | kayak | JNK | 0.371 | 0.371 | — | — | — | — |
| GB12004 | FBgn0001291 | Jun | JNK | 0.450 | 0.471 | — | — | — | — |
| GB16422 | FBgn0004864 | hopscotch | J-ST | — | −0.204 | — | — | — | — |
| | | | | — | | — | — | — | — |
| GB19988 | FBgn0025827 | Lysozyme | Immune | 0.315 | −0.498 | 0.386 | | | |
| GB18918 | FBgn0004606 | zfh1 | Immune | — | 0.401 | — | — | — | — |
| | | | Immune | — | — | — | — | — | |
| GB16457 | FBgn0003963 | u-shaped | | | | 0.291 | | 0.371 | — |
| | | | Immune | — | — | — | — | — | |
| GB15719 | FBgn0086899 | tlk | | | | 0.254 | | 0.259 | |
| | | | Immune | — | | — | — | — | — |
| GB13670 | FBgn0043550 | Tsp68C | | 0.708 | −1.267 | 0.881 | | | |
| | | | Immune | — | | — | — | — | — |
| GB12465 | FBgn0003896 | tailup | | | −0.455 | 0.300 | | | |
| | | | Immune | — | | — | | — | — |
| GB11411 | FBgn0004837 | Su(H) | | | −0.214 | 0.257 | — | | |
| | | | Immune | | | — | — | — | — |
| GB12280 | FBgn0039141 | spastin | | — | −0.318 | | | | 0.261 |
| | | | Immune | — | | — | — | — | — |
| GB16613 | FBgn0011823 | Pendulin | | 0.276 | −0.312 | 0.297 | | | |
| | | | Immune | — | | | — | — | — |
| GB12373 | FBgn0010247 | Parp | | 0.241 | −0.420 | — | | | |
| | | | Immune | — | | — | — | — | — |
| GB10718 | FBgn0085432 | pangolin | | | −0.230 | 0.223 | | | |
| GB17628 | FBgn0262738 | norpA | Immune | — | 0.266 | — | — | — | — |
| GB12005 | FBgn0004657 | mys | Immune | — | — | — | 0.255 | — | 0.251 |
| | | | Immune | — | | — | — | — | — |
| GB10124 | FBgn0013576 | mustard | | | −0.264 | 0.281 | 0.502 | 0.519 | |
| | | | Immune | — | | — | — | | — |
| GB13202 | FBgn0040324 | Ephrin | | | −0.336 | 0.295 | | — | |
| GB19881 | FBgn0027066 | Eb1 | Immune | — | | — | — | — | — |
| | | | | | | 0.208 | | 0.201 | |
| GB12454 | FBgn0243514 | eater | Immune | — | 1.555 | | — | — | — |
| GB13459 | FBgn0031464 | Duox | Immune | 0.791 | 0.619 | 0.498 | — | — | — |
| | | | Immune | — | | — | — | — | — |
| GB19168 | FBgn0011764 | Dsp1 | | 0.222 | −0.355 | 0.216 | | | |
| | | | Immune | | | | — | — | |
| GB14446 | FBgn0259099 | dcx-emap | | — | −0.440 | — | 0.679 | | — |
| | | | Immune | | | | — | — | — |
| GB17018 | FBgn0087011 | CG41520 | | 0.499 | 0.461 | 0.503 | | | |
| | | | | — | | | — | — | — |
| GB14317 | FBgn0021764 | sidekick | IG | | −0.215 | — | | | |
| | | | | — | | — | — | — | — |
| GB19895 | FBgn0003117 | pannier | GATA | | −0.760 | 0.512 | | | |

^{a} Honeybee gene ID according to the Apis mellifera Official Gene Set 1 [30]. Honeybee immune-related genes included in the analysis were either those described in [33] or the honeybee homologues of the Drosophila melanogaster genes associated with Gene Ontology Biological Process term “Immune System Process” GO:0002376.
^{b} Drosophila melanogaster homologue showing highest similarity in BLAST.
^{c} Drosophila melanogaster gene name according to FlyBase.
^{d} Pathway or category of gene if known (Toll-Toll signalling pathway; SP-serine protease; Scav-Scavenger receptor A; Notch-Notch signalling pathway; JNK-JNK signalling pathway; J-ST-JAK-STAT signalling pathway; IG-IG Superfamily Genes; GATA-GATA transcription factor), Immune-Immune system process gene.

TABLE 4
Summary of the NGS libraries and consensus viral sequences from individual honeybees from
Varroa-infested colony. RNA-seq libraries were produced using poly(A) RNA extracts. The reads
were aligned to the reference full-length DWV and VDV-1 sequences, GeneBank Accession
numbers GU109335 and AY251269 respectively, using “ --very-sensitive-local”
option which allowed highest number of mismatches. The aligned reads were used to generate
consensus nucleotide sequences. The assembled viral sequences showed highest identity with the
DWV-VDV-1 recombinant clone identified in the sampled colony (GenBank Accession
number KJ437447) and the group VH sequences (e.g. GenBank Accession number JX661656).

| | | | | Corrected | | | | | |
|---|---|---|---|---|---|---|---|---|---|
| | | | | Average | | | | | |
| | | | | Shannons's | | | | | |
| | | | | diversity index | | | | | |
| Sample | 1 | 2 | 3 | 4 | 5 | 6 | 7 | 8 | 9 |
| INJ4 (SEQ | 10199537 | 755669 | 7.409% | 0.01710 | 0.01470 | 99.16% | 99.83% | 98.50% | 100% |
| ID NO: 60) | | | | | | | | | |
| INJ5 (SEQ | 11277902 | 1367858 | 12.129% | 0.01575 | 0.01305 | 99.16% | 99.83% | 98.50% | 100% |
| ID NO: 61) | | | | | | | | | |
| INJ6 (SEQ | 10253990 | 922841 | 9.000% | 0.01610 | 0.01375 | 99.16% | 99.83% | 98.50% | 100% |
| ID NO: 62) | | | | | | | | | |
| E7 (SEQ ID | 8919720 | 6382279 | 71.552% | 0.01000 | 0.00980 | 99.16% | 99.83% | 98.50% | 100% |
| NO: 56) | | | | | | | | | |
| E8 (SEQ ID | 9998633 | 6767911 | 67.688% | 0.01155 | 0.01330 | 98.59% | 99.59% | 99.47% | 100% |
| NO: 57) | | | | | | | | | |
| E10 (SEQ ID | 8900999 | 5145729 | 57.811% | 0.01450 | 0.01290 | 99.67% | 99.76% | 99.85% | 100% |
| NO: 58) | | | | | | | | | |
| E11 (SEQ ID | 9556537 | 8015413 | 83.874% | 0.01765 | 0.02120 | 99.17% | 99.79% | 98.57% | 100% |
| NO: 59) | | | | | | | | | |

1 = Total NGS reads
2 = Total DWV and VDV-1 reads
3 = Proportion of DWV and VDV-1 reads
4 = CP region
5 = NS region
6 = Nucleotide identity with KJ437447 (SEQ ID NO: 52)
7 = Amino acid identity with KJ437447 ORF (positions 1145-9826)
8 = Nucleotide identity with VH group sequence JX661656 (positions 4926-5255)
9 = Amino acid identity with VH group sequence JX661656

TABLE 5
Oligonucleotides used in this study.
In addition to the oligonucleotides in Table 1,
the following oligonucleotides were also used.

| SEQ ID NO: | Sequence | Description |
|---|---|---|
| 63 | AGGAATGGAAGCTTGCGGTA | Honeybee β-actin, F |
| 64 | AATTTTCATGGTGGATGGTGC | Honeybee β-actin, R |
| 65 | CGGGAGACGCCAGGTTAG | AFB-P. larvae, F |
| 66 | TTCTTCCTTGGCAACAGAGC | AFB-P. larvae, R |
| 67 | TGTTGTTAGAGAAGAATAGGGGAA | EFB-M. plutonius, F qPCR, |
| 68 | CGTGGCTTTCTGGTTAGA | EFB-M. plutonius, R qPCR, |
| 69 | CAAAAAAACTCGTCATATGTTGCCAACTG | Honeybee Rp49 (GB10903), F |
| 70 | GCATCATTAAAACTTCCAGTTCCTTG | Honeybee Rp49 (GB10903), R |
| 71 | GTCATAGCGATCGTTTTCGCTG | Honeybee Vago (GB10896), F |
| 72 | GCTATAATACGACTCACTATAGGGCAATTAGGGAATGCAGC | Honeybee Vago (GB10896), R |

TABLE 6
Summary of the small RNA sequencing in the experimental groups. The single read libraries were aligned
using Bowtie [37] to Apis mellifera miRNA [36], and to the reference full-length DWV and VDV-1
sequences, GenBank Accession numbers GU109335 and AY251269 respectively.

| Experimental group | C | NV | NV | VL | VL | VH | VH |
|---|---|---|---|---|---|---|---|
| Small RNA library | Y1 | YN2 | YN6 | YL4 | YL8 | YH3 | YH7 |
| ID (as in ArrayExpress | | | | | | | |
| E-MTAB-1671) | | | | | | | |
| Total reads | 11087883 | 16459758 | 13102754 | 28406811 | 32232127 | 26047266 | 35064538 |
| Total DWV and VDV-1 | 540 | 1001 | 828 | 9844 | 11513 | 900332 | 1198197 |
| reads miRNA reads | 1585149 | 2655959 | 2084357 | 51108154 | 5572315 | 3160162 | 4179594 |
| Proportion of miRNA reads | 14.3% | 16.1% | 15.9% | 18.0% | 17.3% | 12.1% | 11.9% |
| DWV and VDV-1 reads | 0.341 | 0.377 | 0.397 | 1.926 | 2.066 | 284.901 | 286.678 |
| per 1000 miRNAs reads | | | | | | | |
| Sense DWV and VDV-1 | 75% | 81% | 74% | 73% | 73% | 79% | 79% |
| reads of total viral | | | | | | | |
| Antisense DWV and | 25% | 18% | 26% | 27% | 27% | 21% | 21% |
| VDV-1 reads of total viral | | | | | | | |
| 18 nt sense reads | 12 | 30 | 32 | 141 | 134 | 10553 | 14064 |
| 19 nt sense reads | 10 | 42 | 29 | 200 | 220 | 17273 | 22713 |
| 20 nt sense reads | 38 | 62 | 47 | 488 | 561 | 46599 | 61970 |
| 21 nt sense reads | 78 | 135 | 94 | 1467 | 1697 | 160157 | 216985 |
| 22 nt sense reads | 171 | 275 | 230 | 3941 | 4649 | 401273 | 532019 |
| 23 nt sense reads | 26 | 55 | 50 | 416 | 501 | 32333 | 43047 |
| 24 nt sense reads | 17 | 28 | 21 | 106 | 132 | 7292 | 9616 |
| 25 nt sense reads | 7 | 43 | 24 | 99 | 97 | 5656 | 7592 |
| 26 nt sense reads | 9 | 29 | 19 | 85 | 76 | 5804 | 7938 |
| 27 nt sense reads | 9 | 25 | 15 | 81 | 85 | 5933 | 7952 |
| 28 nt sense reads | 10 | 29 | 23 | 68 | 85 | 5653 | 7350 |
| 29 nt sense reads | 7 | 27 | 14 | 61 | 56 | 5839 | 7659 |
| 30 nt sense reads | 13 | 26 | 17 | 61 | 92 | 6444 | 8563 |
| 18 nt antisense reads | 2 | 2 | 3 | 42 | 47 | 2184 | 3049 |
| 19 nt antisense reads | 4 | 9 | 5 | 80 | 1271 | 5705 | 7818 |
| 20 nt antisense reads | 9 | 15 | 31 | 246 | 302 | 16295 | 21826 |
| 21 nt antisense reads | 33 | 55 | 31 | 607 | 702 | 45594 | 60388 |
| 22 nt antisense reads | 78 | 102 | 122 | 1484 | 1774 | 111186 | 146427 |
| 23 nt antisense reads | 7 | 3 | 12 | 155 | 145 | 7581 | 9989 |
| 24 nt antisense reads | 0 | 3 | 4 | 6 | 19 | 553 | 707 |
| 25 nt antisense reads | 0 | 1 | 0 | 2 | 9 | 131 | 174 |
| 26 nt antisense reads | 0 | 0 | 1 | 3 | 1 | 64 | 90 |
| 27 nt antisense reads | 0 | 1 | 1 | 1 | 0 | 46 | 56 |
| 28 nt antisense reads | 0 | 2 | 2 | 2 | 1 | 51 | 70 |
| 29 nt antisense reads | 0 | 0 | 1 | 1 | 0 | 54 | 64 |
| 30 nt antisense reads | 0 | 2 | 0 | 1 | 1 | 79 | 71 |
| DWV coverage (nt) | 9434 | 12934 | 10327 | 114715 | 138700 | 9906028 | 13212790 |
| VDV-1 coverage (nt) | 6715 | 11507 | 9123 | 123321 | 142988 | 10850253 | 14609501 |
| Coverage ratio, | 1.405 | 1.124 | 1.132 | 0.930 | 0.970 | 0.913 | 0.904 |
| DWV/VDV-1 | | | | | | | |

The single read libraries were aligned using Bowtie to the Apis mellifera miRNA and to the reference full-length DWV and VDV-1 sequences, GeneBank Accession numbers GU109335 and AY251269 respectively.

TABLE 7
Correlation of virus-specific siRNA coverage between
experimental groups. Pearson correlations, P < 0.001 are shown.
The small RNA libraries determined by high-throughput
sequencing were aligned to the DWV or VDV-1 sequences
(GenBank Accession numbers GU109335 and AY251269
respectively) using bowtie [37]. All reads VDV-1 or
DWV pileup values numbers of the DWV- and
VDV-1-specific small RNA reads, up to 3
mismatches were allowed for the 18 nt seed region.
DWV-specific siRNAs

| | C-1 | NV-1 | NV-2 | VL-1 | VL-2 | VH-1 |
|---|---|---|---|---|---|---|
| NV-1 | 0.666 | | | | | |
| NV-2 | 0.593 | 0.706 | | | | |
| VL-1 | 0.786 | 0.768 | 0.737 | | | |
| VL-2 | 0.775 | 0.770 | 0.748 | 0.958 | | |
| VH-1 | 0.768 | 0.764 | 0.732 | 0.957 | 0.962 | |
| VH-2 | 0.765 | 0.751 | 0.726 | 0.955 | 0.963 | 0.997 |

VDV-1-specific siRNAs

| | C-1 | NV-1 | NV-2 | VL-1 | VL-2 | VH-1 |
|---|---|---|---|---|---|---|
| NV-1 | 0.526 | | | | | |
| NV-2 | 0.399 | 0.558 | | | | |
| VL-1 | 0.651 | 0.706 | 0.662 | | | |
| VL-2 | 0.627 | 0.713 | 0.646 | 0.938 | | |
| VH-1 | 0.639 | 0.724 | 0.683 | 0.960 | 0.945 | |
| VH-2 | 0.635 | 0.726 | 0.688 | 0.962 | 0.950 | 0.996 |

TABLE 8
Differential expression of putative homeobox genes in the contrasts.

| BeeBase | Drosophila | | Fold change in contrast (Log2 value) | | | | | |
|---|---|---|---|---|---|---|---|---|
| ID | homolog ID | Gene | C to NV | C to VH | C to VL | NV to VH | NV to VL | VL to VH |
| GB18585 | FBgn0000099 | apterous | — | −0.57201 | — | — | — | — |
| GB15698 | FBgn0000625 | eyegone | −0.2951224 | −0.5625597 | — | — | — | — |
| GB10341 | FBgn0000015 | Abdominal B | — | −0.591549 | −0.3959795 | −0.358176 | — | — |
| GB15837 | FBgn0000611 | extradenticle | — | −0.4891224 | −0.4226791 | −0.3704909 | — | — |
| GB14165 | FBgn0011701 | reversed | — | 0.3483972 | — | — | — | 0.3480016 |
| | | polarity | | | | | | |

### CONCLUSIONS

Without proper management Varroa has a devastating effect on honeybee colony viability and consequent honey production and pollination services. We show here that the markedly elevated levels of DWV-like viruses in Varroa-exposed honeybee pupae are likely attributable to the direct inoculation of a specific virus, DWV^{V}, by Varroa to haemolymph. Repeated cycles of Varroa-replication within an infested colony would preferentially amplify DWV^{V}, potentially resulting in it becoming the predominant virus present, transferred both by Varroa and per os. Further studies will be required to determine whether such a virus, if sufficient were ingested, would also cause symptomatic infection. Oral susceptibility to a virulent form of DWV may also explain reported cases of deformed wing disease symptoms seen in Varroa-free colonies in Hawaii [20] and Scotland (Andrew Abrahams, pers. comm.), but may also reflect genetic variation and the presence of particularly susceptible pupae in the colony.

Our study demonstrates that a proportion of Varroa-exposed pupae (the VL group) do not exhibit elevated levels of the near-clonal DWV^{V} recombinant (FIG. 12). Further in vitro studies will be required to determine whether these are naturally resistant—and therefore form the basis for genome wide association studies of the genetic determinants of virus resistance—or if they reflect the stochastic nature of the transmission event from the mite.

### Materials and Methods

### Honeybees, Frame Transfer Experiment and Sampling

This study was based around an experiment in which a brood frame containing newly hatched larvae from a Varroa-free colony was introduced into a Varroa-infested colony. The larvae were left to develop within the Varroa-infested colony, and pupae were collected 11 days later from capped brood cells at the purple eye stage and analysed using a range of molecular methods. The Varroa-free honeybee (Apis mellifera) colony with a naturally-mated one-year-old queen was imported from Colonsay, Scotland, an island with no historic reports of Varroa incidence and no imports of honeybees from Varroa-infested areas. This allowed us to exclude the presence of DWV strains associated with Varroa mite infestation. As a source of Varroa mites and the mite-associated DWV strains, we selected a Warwickshire honeybee colony, heavily infested with Varroa and having high DWV levels in honeybees and mites. The Varroa-free and Varroa-infested colonies were contained in separate mesh flight cages (dimensions: 6 meters long, 2.5 meters wide, 2 meters high) and maintained on an artificial diet of sugar syrup and pollen. The pollen was imported from Varroa-free Australia to exclude possible contamination with Varroa-associated viruses through foraged food and was pre-screened by PCR before use for DWV-like viruses. In order to minimise possible effects on honeybee gene expression due to the differences in nutrition, both the control Varroa-free and the Varroa-infested colonies were maintained in flight cages in the same apiary (at the University of Warwick, UK) and were fed on the artificial diet for two months before the start of the frame transfer experiments. Neither colony was treated with miticides.

The experimental infestation, summarised in FIG. 2, was conducted on 4^{th}-15^{th} August 2011. As stated previously, it involved the transfer of a brood frame, which contained newly hatched honeybee worker larvae (on day 4 of development), from the Varroa-free to the Varroa-infested colony. As a result, the transferred larvae were exposed to Varroa-selected DWV-like viruses in brood food delivered by the nurse honeybees of the Varroa-infested colony for five days before brood cell capping on day nine of development (FIG. 2, Treatment 1, Oral DWV infection). Honeybee larvae were left to develop in the capped cells for six days and then were sampled as pupae on day 15, when they had reached the purple-eye stage of development [74]. A proportion of these brood cells were naturally infested with Varroa and hence contained pupae that were subjected to mite feeding (FIG. 2, Treatment 2, Mite feeding). We sampled Varroa-infested pupae and the mites associated with individual pupae, with mite feeding confirmed by the presence of the mother mite and at least one protonymph [27]. Control pupae at the same developmental stage were sampled from the Varroa-free hive at the same time. A colony from a separate apiary in Warwickshire that exhibited Varroa mite infestation for over a year was sampled in August 2013 to assess the virus populations in colonies with established Varroa infestation.

The pupae and the Varroa mites associated with each infested pupa were individually snap frozen in liquid nitrogen immediately after being removed from brood cells and stored at −80° C. prior to total RNA extraction. For total RNA extraction, whole individual honeybee pupae were ground to fine powder in liquid nitrogen, and half of the powder used for RNA extraction, carried out using 1 mL of Trizol Reagent (Invitrogen) according to the manufacturer's instructions. Total RNA extraction from Varroa mites was carried out using RNeasy spin columns (Qiagen RNeasy Plant Mini kit).

Virus purification from honeybee material and extraction of the viral genomic RNA from virus particles were carried out as described previously [15].

### Microarray Transcriptional Profiling and Statistical Analysis

For genome-wide analysis of the honeybee transcriptome total RNA preparations from eight individual honeybees from each of the four experimental groups (32 honeybees in total) were purified further using RNeasy Plant Mini kit spin columns (Qiagen). RNA concentration, purity and integrity were assessed using a 2100 Bioanalyzer and an RNA 6000 LabChip (Agilent Technologies). The probe preparation, hybridization and scanning were carried out according to the Agilent instructions, essentially as in [75]. Total RNA extracts from an individual honeybee were used to produce Cy3- and Cy5-labelled aRNA samples using a Low Input RNA fluorescent linear amplification kit (Agilent Technologies), according to the manufacturer's instructions. The Cy3- and Cy5-labelled samples were used in a two-colour dye-balanced loop design [30,31] for a genome-wide analysis of the honeybee transcriptome with the custom expression oligonucleotide microarray. Four slides, each with eight two-channel arrays were employed, allowing two replicates per sample, one green and one red. Different treatment groups were allocated to the green and red channels in each array; the loop design ensured that each sample was indirectly compared with all other samples. The array, in 60K×8 format, included 60 nt oligonucleotides specific to 10,157 transcripts of the Apis mellifera Official Gene Set 1, OGS1 [32]; the array also contained probes to all the honeybee RNA viruses known to date. Each probe was replicated five times to enable robust statistical analysis. Sequences of the probes of the honeybee whole genome expression microarray, a 60-mer oligonucleotidide array based on the Apis mellifera transcriptome (OGS1) and Apis mellifera fungal and viral pathogens (Agilent ID: 027104, SurePrint G3 Custom GE 8×60K), are available in the ArrayExpress database (www.ebi.ac.uk/arrayexpress) under accession number A-MEXP-2251. Following hybridisation, the microarrays were scanned using Agilent Technologies G2565CA Scanner and the fluorescence intensity data were processed using feature extraction software (Agilent Technologies). Cy3 and Cy5 fluorescence intensities for each spot were measured as values of green and red pixels respectively. The details of the array experiment design, sample description, and microarray data are available in the ArrayExpress database (www.ebi.ac.uk/arrayexpress) [76] under accession number E-MTAB-1285. One array failed (assigned to VL green and NV red) leaving 62 channels for final analysis.

For additional confirmation we conducted qRT-PCR analysis with primers specific to Paenibacillus larvae ssp and Melissococcus plutonius (Table 5), the causal agents of American foulbrood and European foulbrood, respectively, which showed that the samples were free of detectable levels of these bacterial pathogens.

The unprocessed intensity scanning values were both within-array and between-array normalized using the linear model based Limma R package [77]. Differentially expressed (DE) genes in all six possible contrasts were found using Limma (via function “lmscFit” incorporating intraspot correlation) and also the R GaGa package for gamma-gamma Bayesian hierarchical modeling [78-80]. A gene was considered as differentially expressed (DE) in a given contrast (using a t-statistic moderated across genes) when the average expression exceeded 6.0, the fold change exceeded 14%, the Limma analysis p-value adjusted for multiple genes was less than 0.05 and the posterior probability determined by GaGa was above 0.6. Microarray results were validated by qRT-PCR using a set of primers for certain honeybee genes and DWV (Tables 1 and 5).

For Gene Ontology (GO) analysis a three-stage process was used. Genes in the latest A. mellifera genome annotation, Amel_{—}4.0 (http://hymenopteragenome.org/beebase), corresponding to genes in A. mellifera OGS1 were found using protein blast. GO terms associated with Amel_{—}4.0 genes were then obtained using Blast2GO [81] with the SwissProt database option. Finally, over- and under-represented GO terms in the sets of DE honeybee OGS1 genes in each contrast were obtained with BiNGO, using a hypergeometric test, a Benjamini and Hochberg FDR correction and a significance level of 0.05 [82].

For Principal Component Analysis (PCA), the significant DE genes in all six contrasts were pooled and ranked by their adjusted p-value. The 60 with the lowest adjusted p-value were selected, all of which appeared in the contrast C to VH; the other contrasts' contributions were 35 (C to VL), 21 (NV to VH), 19 (C to NV), 4 (NV to VL) and 11 (VL to VH) genes. Principal components of the expression profiles across the 62 microarray channels were found and (the first two) plotted using the princomp and biplot functions in R [83].

### Next Generation Sequencing of Small RNA Libraries

For high throughput sequencing of small RNA, we pooled equal amounts of the Trizol-extracted total RNA from individual honeybees and isolated the 15 to 40 nt RNA fraction, which was separated using denaturing polyacrylamide gel. The RNA pools were ligated to the oligonucleotide adapters, reverse-transcribed and amplified using the TruSeq Small RNA Sample Prep Kit (Illumina small RNA kit). The libraries were sequenced using the Illumina HighSeq 2000 platform, producing 15-25 million reads per libraries (GATC-Biotech, Germany). The small RNA NGS sequencing data are available in the ArrayExpress database (www.ebi.ac.uk/arrayexpress) [76] under accession number E-MTAB-1671. The reads were cropped to remove adapter sequences and aligned to reference viral and miRNA sequences using Bowtie [37]. Samtools mpileup was used to produce the siRNA and miRNA coverage profiles.

### Characterization of Viral RNA

Real-time reverse transcription PCR was carried out essentially as in [15]. In brief, RNA extracts were treated with DNAse, then purified DNA-free total RNA preparations were used as a template to produce cDNA using random primer and Superscript III reverse transcriptase (Invitrogen). The cDNA samples produced were used for real-time PCR quantification of the DWV or host transcripts using SYBR green mix (Agilent Technologies). Oligonucleotide primers are summarized in Tables 1 and 5.

For strand-specific quantification of viral RNA of DWV and VDV-1 types reverse transcription was carried out at 50° C. using Superscript III reverse transcriptase (Invitrogen) and the tagged primers designed to anneal to the negative strands RNA of DWV or VDV-1, primers 389 and 391 respectively (SEQ ID NOs: 50 and 51 in Table 1). The qPCR step was carried out using corresponding DWV or VDV-1 specific primers in negative polarity (Table 1, SEQ ID NOs: 15 and 16) and primer 388 identical to the sequence of the tag (Table 1, SEQ ID NO: 49).

Amplification of the cDNA fragments corresponding to the central region of DWV genomic RNA was carried out by nested PCR using GoTaq PCR mix (Promega) and primers 155 and 156 (SEQ ID NOs: 23 and 24 in Table 1) using the cDNA extracted from the honeybees and the mites, pooled according to their treatment groups. The outside PCR primers were designed to amplify all known DWV-like sequences. For each first round reaction we carried four second round amplification reactions using VDV-1- or DWV-specific primers, 151-154 (SEQ ID NOs: 23 to 26 in Table 1), which allowed distinction of VDV-1-type and DWV-type CP and NS regions, thereby enabling amplification of all potential combinations, even those present at very low levels. The PCR fragments were cloned into pGemT-Easy (Promega) and sequenced using the Sanger dideoxy method. GenBank accession numbers for the reported sequences are JX661628-JX661712 and KC249926-KC249933. The full-length cDNA of DWV, GenBank accession number KJ437447, was amplified by RT-PCR using primers specific to the published termini of DWV and VDV-1 RNA and cloned into the pCR-TOPO-XL vector (Invitrogen) as described in [15]. The sequences were aligned using CLUSTAL X [84], and phylogenetic analysis of the sequences was carried out using the PHYLIP package [85].

For the next generation sequencing of RNA, a series of overlapping cDNA fragments were produced using viral RNA or total RNA preparations using the set of primers designed to the sequences of the genomic RNA conserved among DWV, VDV-1 and KV (Table 1). The fragments were pooled and libraries of paired-end reads (101 nt.), about 5 million per sample, were generated using an Illumina HiSeq 2000 (GATC-Biotech). The virus genomic RNA NGS sequencing data are available in the ArrayExpress database (www.ebi.ac.uk/arrayexpress)[76] under accession number E-MTAB-1675.

The next generation sequencing of the poly(A) RNA fraction (RNA-seq) of the total RNA preparations isolated from the honeybees was carried out using Illumina HiSeq 2000 (GATC-Biotech) protocol, with about 10 million 101 nucleotide-long reads generated for each sample. The RNA-seq sequencing data are available in the EBI Sequence Read Archive [86] under accession number PRJEB5249. This RNA-seq dataset was used to calculate Shannon's diversity index values of DWV populations using the following procedure. First we selected the reads aligning to the reference DWV and VDV-1 sequences (GenBank Accession numbers GU109335 and AY251269 respectively) from the original RNA-seq libraries using Bowtie. To take into account the effect of difference in coverage of low virus levels and high virus level RNA-seq libraries we used two approaches, (i) correction for NGS error for complete libraries ([44]) and (ii) multiple sampling. For the latter we produced five samples of 3285 reads (the lowest number of the viral reads among the libraries), which were aligned using Bowtie to the reference DWV and VDV-1 sequences, and the NGS nucleotide pileups were then generated for each nucleotide position of the reference sequences using samtools. Shannon's diversity index of the aligned nucleotides was calculated for each position in the reference sequence. Then, the average Shannon's index values were calculated for the selected regions in the reference genomes for each sample. The averages values of and standard deviation of five samples were used in the statistical analysis.

### REFERENCES FOR EXAMPLE 8

1. Sorrell I, White A, Pedersen A B, Hails R S, Boots M (2009) The evolution of covert, silent infection as a parasite strategy. Proceedings of the Royal Society B-Biological Sciences 276: 2217-2226.
2. Lambrechts L, Scott T W (2009) Mode of transmission and the evolution of arbovirus virulence in mosquito vectors. Proceedings of the Royal Society B-Biological Sciences 276: 1369-1378.
3. Weiss R A (2002) Virulence and pathogenesis. Trends in Microbiology 10: 314-317.
4. Boots M, Greenman J, Ross D, Norman R, Hails R, et al. (2003) The population dynamical implications of covert infections in host-microparasite interactions. Journal of Animal Ecology 72: 1064-1072.
5. Rigaud T, Perrot-Minnot M-J, Brown M J F (2010) Parasite and host assemblages: embracing the reality will improve our knowledge of parasite transmission and virulence. Proceedings of the Royal Society B-Biological Sciences 277: 3693-3702.
6. Woolhouse M E J, Taylor L H, Haydon D T (2001) Population biology of multihost pathogens. Science 292: 1109-1112.
7. Klein A-M, Vaissiere B E, Cane J H, Steffan-Dewenter I, Cunningham S A, et al. (2007) Importance of pollinators in changing landscapes for world crops. Proceedings of the Royal Society B-Biological Sciences 274: 303-313.
8. Martin S J (2001) The role of Varroa and viral pathogens in the collapse of honeybee colonies: a modelling approach. Journal of Applied Ecology 38: 1082-1093.
9. Genersch E, Aubert M (2010) Emerging and re-emerging viruses of the honey bee (Apis mellifera L.). Veterinary Research 41.
10. Dainat B, Evans J D, Chen Y P, Gauthier L, Neumann P (2012) Dead or Alive: Deformed Wing Virus and Varroa destructor Reduce the Life Span of Winter Honeybees. Applied and Environmental Microbiology 78: 981-987.
11. Vanbergen A J, Baude M, Biesmeijer J C, Britton N F, Brown M J F, et al. (2013) Threats to an ecosystem service: pressures on pollinators. Frontiers in Ecology and the Environment 11: 251-259.
12. Lanzi G, De Miranda J R, Boniotti M B, Cameron C E, Lavazza A, et al. (2006) Molecular and biological characterization of deformed wing virus of honeybees (Apis mellifera L.). Journal of Virology 80: 4998-5009.
13. de Miranda J R, Genersch E (2010) Deformed wing virus. Journal of Invertebrate Pathology 103: S48-S61.
14. Ongus J R, Peters D, Bonmatin J M, Bengsch E, Vlak J M, et al. (2004) Complete sequence of a picorna-like virus of the genus Iflavirus replicating in the mite Varroa destructor. Journal of General Virology 85: 3747-3755.
15. Moore J, Jironkin A, Chandler D, Burroughs N, Evans D J, et al. (2011) Recombinants between Deformed wing virus and Varroa destructor virus-1 may prevail in Varroa destructor-infested honeybee colonies. The Journal of general virology 92: 156-161.
16. Zioni N, Soroker V, Chejanovsky N (2011) Replication of Varroa destructor virus 1 (VDV-1) and a Varroa destructor virus 1-deformed wing virus recombinant (VDV-1-DWV) in the head of the honey bee. Virology 417: 106-112.
17. Fujiyuki T, Takeuchi H, Ono M, Ohka S, Sasaki T, et al. (2004) Novel insect picorna-like virus identified in the brains of aggressive worker honeybees. Journal of Virology 78: 1093-1100.
18. Yang X L, Cox-Foster D L (2005) Impact of an ectoparasite on the immunity and pathology of an invertebrate: Evidence for host immunosuppression and viral amplification. Proceedings of the National Academy of Sciences of the United States of America 102: 7470-7475.
19. Gregory P G, Evans J D, Rinderer T, de Guzman L (2005) Conditional immune-gene suppression of honeybees parasitized by Varroa mites. Journal of Insect Science 5.
20. Martin S J, Highfield A C, Brettell L, Villalobos E M, Budge G E, et al. (2012) Global Honey Bee Viral Landscape Altered by a Parasitic Mite. Science 336: 1304-1306.
21. Navajas M, Migeon A, Alaux C, Martin-Magniette M L, Robinson G E, et al. (2008) Differential gene expression of the honey bee Apis mellifera associated with Varroa destructor infection. Bmc Genomics 9.
22. Johnson R M, Evans J D, Robinson G E, Berenbaum M R (2009) Changes in transcript abundance relating to colony collapse disorder in honey bees (Apis mellifera). Proceedings of the National Academy of Sciences of the United States of America 106: 14790-14795.
23. Nazzi F, Brown S P, Annoscia D, Del Piccolo F, Di Prisco G, et al. (2012) Synergistic Parasite-Pathogen Interactions Mediated by Host Immunity Can Drive the Collapse of Honeybee Colonies. Plos Pathogens 8.
24. Merkling S H, van Rij R P (2013) Beyond RNAi: Antiviral defense strategies in Drosophila and mosquito. Journal of Insect Physiology 59: 159-170.
25. Kemp C, Imler J-L (2009) Antiviral immunity in drosophila. Current Opinion in Immunology 21: 3-9.
26. Goic B, Vodovar N, Mondotte J A, Monot C, Frangeul L, et al. (2013) RNA-mediated interference and reverse transcription control the persistence of RNA viruses in the insect model Drosophila. Nature Immunology 14: 396-403.
27. Rosenkranz P, Aumeier P, Ziegelmann B (2010) Biology and control of Varroa destructor. Journal of Invertebrate Pathology 103: S96-S119.
28. Tentcheva D, Gauthier L, Jouve S, Canabady-Rochelle L, Dainat B, et al. (2004) Polymerase Chain Reaction detection of deformed wing virus (DWV) in Apis mellifera and Varroa destructor. Apidologie 35: 431-439.
29. Martin S J, Ball B V, Carreck N L (2013) The role of deformed wing virus in the initial collapse of varroa infested honey bee colonies in the U K. Journal of Apicultural Research 52.
30. Vinciotti V, Khanin R, D'Alimonte D, Liu X, Cattini N, et al. (2005) An experimental evaluation of a loop versus a reference design for two-channel microarrays. Bioinformatics 21: 492-501.
31. Bailey R A (2007) Designs for two-colour microarray experiments. Journal of the Royal Statistical Society Series C-Applied Statistics 56: 365-394.
32. Weinstock G M, Robinson G E, Gibbs R A, Worley K C, Evans J D, et al. (2006) Insights into social insects from the genome of the honeybee Apis mellifera. Nature 443: 931-949.
33. Ashburner M, Ball C A, Blake J A, Botstein D, Butler H, et al. (2000) Gene Ontology: tool for the unification of biology. Nature Genetics 25: 25-29.
34. Evans J D, Aronstein K, Chen Y P, Hetru C, Imler J L, et al. (2006) Immune pathways and defence mechanisms in honey bees Apis mellifera. Insect Molecular Biology 15: 645-656.
35. Zou Z, Lopez D L, Kanost M R, Evans J D, Jiang H (2006) Comparative analysis of serine protease-related genes in the honey bee genome: possible involvement in embryonic development and innate immunity. Insect Molecular Biology 15: 603-614.
36. Chen X, Yu X, Cai Y, Zheng H, Yu D, et al. (2010) Next-generation small RNA sequencing for microRNAs profiling in the honey bee Apis mellifera. Insect Molecular Biology 19: 799-805.
37. Langmead B, Trapnell C, Pop M, Salzberg S L (2009) Ultrafast and memory-efficient alignment of short DNA sequences to the human genome. Genome Biology 10.
38. Yue C, Genersch E (2005) RT-PCR analysis of Deformed wing virus in honeybees (Apis mellifera) and mites (Varroa destructor). Journal of General Virology 86: 3419-3424.
39. Moeckel N, Gisder S, Genersch E (2011) Horizontal transmission of deformed wing virus: pathological consequences in adult bees (Apis mellifera) depend on the transmission route. Journal of General Virology 92: 370-377.
40. Wood G, Ryabov E, Fannon J, Moore J, Evans D J, et al. (2014) MosaicSolver: a tool for determining recombinants of viral genomes from pileup data. Nucleic Acids Research (Submitted).
41. Wang Z, Gerstein M, Snyder M (2009) RNA-Seq: a revolutionary tool for transcriptomics. Nature Reviews Genetics 10: 57-63.
42. Akhter S, Bailey B A, Salamon P, Aziz R K, Edwards R A (2013) Applying Shannon's information theory to bacterial and phage genomes and metagenomes. Scientific Reports 3: 7.
43. Quail M A, Smith M, Coupland P, Otto T D, Harris S R, et al. (2012) A tale of three next generation sequencing platforms: comparison of Ion Torrent, Pacific Biosciences and Illumina MiSeq sequencers. Bmc Genomics 13.
44. Wood G R, Burroughs N, Evans D J, Ryabov E V (2014) Nucleotide diversity analysis: next generation sequencing error correction, hypothesis testing and clonal threshold estimation. Bioinformatics (Preprint).
45. vanEngelsdorp D, Meixner M D (2010) A historical review of managed honey bee populations in Europe and the United States and the factors that may affect them. Journal of Invertebrate Pathology 103: S80-S95.
46. Furst M A, McMahon D P, Osborne J L, Paxton R J, Brown M J F (2014) Disease associations between honeybees and bumblebees as a threat to wild pollinators. Nature 506: 364-366.
47. Koudritsky M, Domany E (2008) Positional distribution of human transcription factor binding sites. Nucleic Acids Research 36: 6795-6805.
48. Doukas T, Sarnow P (2011) Escape from Transcriptional Shutoff during Poliovirus Infection: N F-kappa B-Responsive Genes I kappa Ba and A20. Journal of Virology 85: 10101-10108.
49. Rozovics J M, Chase A J, Cathcart A L, Chou W, Gershon P D, et al. (2012) Picornavirus Modification of a Host mRNA Decay Protein. Mbio 3.
50. Grinde B, Gayorfar M, Hoddevik G (2007) Modulation of gene expression in a human cell line caused by poliovirus, vaccinia virus and interferon. Virology Journal 4.
51. Valanne S, Wang J-H, Ramet M (2011) The Drosophila Toll Signaling Pathway. Journal of Immunology 186: 649-656.
52. Deddouche S, Matt N, Budd A, Mueller S, Kemp C, et al. (2008) The DExD/H-box helicase Dicer-2 mediates the induction of antiviral activity in drosophila. Nature Immunology 9: 1425-1432.
53. Paradkar P N, Trinidad L, Voysey R, Duchemin J-B, Walker P J (2012) Secreted Vago restricts West Nile virus infection in Culex mosquito cells by activating the Jak-STAT pathway. Proceedings of the National Academy of Sciences of the United States of America 109: 18915-18920.
54. Zambon R A, Nandakumar M, Vakharia V N, Wu L P (2005) The Toll pathway is important for an antiviral response in Drosophila. Proceedings of the National Academy of Sciences of the United States of America 102: 7257-7262.
55. Basset A, Khush R S, Braun A, Gardan L, Boccard F, et al. (2000) The phytopathogenic bacteria Erwinia carotovora infects Drosophila and activates an immune response. Proceedings of the National Academy of Sciences of the United States of America 97: 3376-3381.
56. Levy S, Shoham T (2005) The tetraspanin web modulates immune-signalling complexes. Nature Reviews Immunology 5: 136-148.
57. Minakhina S, Tan W, Steward R (2011) JAK/STAT and the GATA factor Pannier control hemocyte maturation and differentiation in Drosophila. Developmental Biology 352: 308-316.
58. Lebestky T, Jung S H, Banerjee U (2003) A serrate-expressing signaling center controls Drosophila hematopoiesis. Genes & Development 17: 348-353.
59. Richards E H, Jones B, Bowman A (2011) Salivary secretions from the honeybee mite, Varroa destructor: effects on insect haemocytes and preliminary biochemical characterization. Parasitology 138: 602-608.
60. Lavine M D, Strand M R (2002) Insect hemocytes and their role in immunity. Insect Biochemistry and Molecular Biology 32: 1295-1309.
61. Hillyer J F, Schmidt S L, Fuchs J F, Boyle J P, Christensen B M (2005) Age-associated mortality in immune challenged mosquitoes (Aedes aegypti) correlates with a decrease in haemocyte numbers. Cellular Microbiology 7: 39-51.
62. Hillyer J F (2009) Transcription in mosquito hemocytes in response to pathogen exposure. Journal of Biology 8: 51.
63. Williams M J (2007) Drosophila hemopoiesis and cellular immunity. Journal of Immunology 178: 4711-4716.
64. Meister M, Lagueux M (2003) Drosophila blood cells. Cellular Microbiology 5: 573-580.
65. McGinnis W, Krumlauf R (1992) Homeobox genes and axial patterning. Cell 68: 283-302.
66. Myles K M, Wiley M R, Morazzani E M, Adelman Z N (2008) Alphavirus-derived small RNAs modulate pathogenesis in disease vector mosquitoes. Proceedings of the National Academy of Sciences of the United States of America 105: 19938-19943.
67. Wang H, Xie J, Shreeve T G, Ma J, Pallett D W, et al. (2013) Sequence Recombination and Conservation of Varroa destructor Virus-1 and Deformed Wing Virus in Field Collected Honey Bees (Apis mellifera). Plos One 8.
68. Chejanovsky N, Ophir R, Schwager M S, Slabezki Y, Grossman S, et al. (2014) Characterization of viral siRNA populations in honey bee colony collapse disorder. Virology 254-255: 176-183.
69. Desai S D, Eu Y J, Whyard S, Currie R W (2012) Reduction in deformed wing virus infection in larval and adult honey bees (Apis mellifera L.) by double-stranded RNA ingestion. Insect Molecular Biology 21: 446-455.
70. Brackney D E, Beane J E, Ebel G D (2009) RNAi Targeting of West Nile Virus in Mosquito Midguts Promotes Virus Diversification. Plos Pathogens 5.
71. Nayak A, Berry B, Tassetto M, Kunitomi M, Acevedo A, et al. (2010) Cricket paralysis virus antagonizes Argonaute 2 to modulate antiviral defense in Drosophila. Nature Structural & Molecular Biology 17: 547-U541.
72. Amdam G V, Simoes Z L P, Guidugli K R, Norberg K, Omholt S W (2003) Disruption of vitellogenin gene function in adult honeybees by intra-abdominal injection of double-stranded RNA. Bmc Biotechnology 3.
73. Wolschin F, Mutti N S, Amdam G V (2011) Insulin receptor substrate influences female caste development in honeybees. Biology Letters 7: 112-115.
74. Whinston M (1987) The biology of the honeybee. Boston, Mass.: Harvard University Press.
75. Bull J C, Ryabov E V, Prince G, Mead A, Zhang C, et al. (2012) A strong immune response in young adult honeybees masks their increased susceptibility to infection compared to older bees. PLoS pathogens 8: e1003083.
76. Rustici G, Kolesnikov N, Brandizi M, Burdett T, Dylag M, et al. (2013) ArrayExpress update-trends in database growth and links to data analysis tools. Nucleic Acids Research 41: D987-D990.
77. Smyth G K (2005) Limma: linear models for microarray data. In: Gentleman R, Carey V, Dudoit S, Irizarry R, Huber W, editors. Bioinformatics and Computational Biology Solutions using R and Bioconductor. New York: Springer pp. 397-420.
78. Kendziorski C M, Newton Mass., Lan H, Gould M N (2003) On parametric empirical Bayes methods for comparing multiple groups using replicated gene expression profiles. Statistics in Medicine 22: 3899-3914.
79. Newton Mass., Kendziorski C M, Richmond C S, Blattner F R, Tsui K W (2001) On differential variability of expression ratios: Improving statistical inference about gene expression changes from microarray data. Journal of Computational Biology 8: 37-52.
80. Rossell D (2009) GAGA: A parsimonious and flexible model for differential expression analysis. Annals of Applied Statistics 3: 1035-1051.
81. Conesa A, Gotz S (2008) Blast2GO: A comprehensive suite for functional analysis in plant genomics. International journal of plant genomics 2008: 619832-619832.
82. Maere S, Heymans K, Kuiper M (2005) BiNGO: a Cytoscape plugin to assess overrepresentation of Gene Ontology categories in Biological Networks. Bioinformatics 21: 3448-3449.
83. Team RDC (2011) R: a language and environment for statistical computing. Vienna, Austria: R Foundation for Statistical Computing.
84. Thompson J D, Gibson T J, Plewniak F, Jeanmougin F, Higgins D G (1997) The CLUSTAL X windows interface: flexible strategies for multiple sequence alignment aided by quality analysis tools. Nucleic Acids Research 25: 4876-4882.
85. Felsenstein J (1989) PHYLIP—Phylogeny inference package (version 3.2). Cladistics 5: 164-166.
86. Leinonen R, Sugawara H, Shumway M, Int Nucleotide Sequence Database C (2011) The Sequence Read Archive. Nucleic Acids Research 39: D19-D21.

1. A polynucleotide comprising a sequence having at least 98% homology to SEQ ID NO: 1, 2, 52, 53, 54, 55, 56, 57, 58, 59, 60, 61 or 62 based on nucleotide identity over its entire length.

2. An oligonucleotide which specifically hybridises to a part of a polynucleotide according to claim 1.

3-4. (canceled)

5. An oligonucleotide according to claim 2, which is a ribonucleic acid (RNA).

6-8. (canceled)

9. An isolated strain of deformed wing virus (DWV) which comprises the varroa destructor virus 1 (VDV-1) capsid proteins (CP) and the DWV non-structural proteins (NS).

10. (canceled)

11. An antibody which specifically binds to an isolated strain of DWV according to claim 9.

12-13. (canceled)

14. A vector comprising-an oligonucleotide according to claim 2, wherein said oligonucleotide is operably linked to a promoter.

15. A vector according to claim 14, wherein said promoter is the honeybee heatshock protein 70 (hsp70) promoter.

16. A vector according to claim 15, wherein said promoter comprises the sequence of SEQ ID NO: 8.

17. A vector according to claim 14, which further comprises retroviral RNA which facilitates integration into the bee genome.

18. (canceled)

19. A composition comprising an oligonucleotide according to claim 2, an antibody according to claim 11 or a vector according to claim 14, and a delivery vehicle.

20-21. (canceled)

22. A method of treating or preventing deformed wing disease in a Varroa mite-infested bee or bee colony, comprising contacting the bee or bee colony with an inhibitor of an isolated strain of DWV according to claim 9.

23. A method according to claim 22, wherein the inhibitor is a peptide, peptidomimetic, antibody, small molecule inhibitor, double-stranded RNA, antisense RNA, aptamer or ribozyme.

24-25. (canceled)

26. A method of treating or preventing in a bee or bee colony an infection, wherein said method comprises contacting at least one bee with an oligonucleotide according to claim 2.

27. (canceled)

28. A method of diagnosing in a bee or bee colony infection with an isolated strain of DWV according to claim 9, comprising determining the presence or absence of the isolated strain of DWV, wherein the presence of the isolated strain of DWV is indicative of the presence of infection with the isolated strain of DWV and wherein the absence of the isolated strain of DWV is indicative of the absence of infection with the isolated strain.

29-31. (canceled)

32. A transgenic bee that is resistant to infection by an isolated strain of DWV, wherein at least one cell of the bee expresses an oligonucleotide according to claim 2.

33. (canceled)

34. A transgenic bee according to claim 32 wherein the oligonucleotide is stably incorporated into the bee genome.

35. (canceled)

36. A method of generating a transgenic queen bee that is resistant to infection by an isolated strain of DWV, comprising
(a) incorporating an oligonucleotide according to claim 2 into the genome of one or more bee germ cells; and
(b) generating the queen bee from said one or more germ cells.

37-39. (canceled)

40. A method of producing a bee or bee colony that is resistant to infection from an isolated strain of DWV, comprising using a transgenic queen bee produced using a method according to claim 36 to generate the bee or bee colony.

41. One or more bee germ cells comprising a vector according to claim 14.

42-43. (canceled)

44. A bee colony wherein at least 50% of the bees within the colony are resistant to Varroa mite-induced deformed wing disease.

45. (canceled)
